(19) United States
(12) Patent Application Publication
(10) Pub. No.: US 2025/0188179 A1
(43) Pub. Date: Jun. 12, 2025

(54) ANTI-CD137 ANTIBODIES AND METHODS OF MAKING AND USING THE SAME

(71) Applicant: ADAGENE PTE. LTD., Singapore (SG)

(72) Inventors: Peter Peizhi LUO, Morgan Hill, CA (US); Fangyong DU, Suzhou, Jiangsu (CN); Guizhong LIU, Fort Lee, NJ (US); Yan LI, San Jose, CA (US)

(73) Assignee: ADAGENE PTE. LTD., Singapore (SG)

(21) Appl. No.: 18/842,313

(22) PCT Filed: Mar. 6, 2023
(86) PCT No.: PCT/CN2023/079843

§ 371 Date: Aug. 28, 2024

(30) Foreign Application Priority Data
Mar. 7, 2022 (WO) PCT/CN2022/079475

(51) Int. Cl. C07K 16/28; A61K 39/00; A61P 35/00; C07K 16/32

(52) U.S. Cl. CPC C07K 16/2878; A61P 35/00; C07K 16/2809; C07K 16/2818; C07K 16/32; A61K 2039/505; C07K 2317/31; C07K 2317/33; C07K 2317/622; C07K 2317/732; C07K 2317/734; C07K 2317/75; C07K 2317/76; C07K 2317/92; C07K 2319/50

(57) ABSTRACT

Provided herein are masked anti-CD137 antibodies, nucleic acids encoding such antibodies, vectors comprising such nucleic acids, and host cells comprising such nucleic acids or vectors. Also provided are methods of producing masked anti-CD137 antibodies. Further provided are methods of treating cancer that comprise administering an effective amount of a masked anti-CD137 antibody as monotherapy or in combination with another therapeutic antibody.

### CROSS REFERENCE TO RELATED APPLICATIONS

This application claims the priority benefit of International Patent Application No. PCT/CN2022/079475, filed Mar. 7, 2022, the contents of which are incorporated herein by reference in their entirety.

### REFERENCE TO AN ELECTRONIC SEQUENCE LISTING

The contents of the electronic sequence listing (695402002241SEQLIST.xml; Size: 126,181 bytes; and Date of Creation: Mar. 3, 2023) are herein incorporated by reference in their entirety.

### FIELD OF THE INVENTION

The present disclosure relates to masked anti-CD137 antibodies that bind to human CD137 and antigen binding fragments thereof, compositions comprising same, and uses thereof in delaying and/or preventing tumor growth.

### BACKGROUND

CD137 (also referred to as CD137 receptor, 4-1BB, TNFRSF9, etc.) is a transmembrane protein of the Tumor Necrosis Factor Receptor Superfamily (TNFRS). Current understanding of CD137 indicates that its expression is generally activation dependent and is present in a broad subset of immune cells including activated NK and NKT cells, regulatory T cells, dendritic cells (DC), stimulated mast cells, differentiating myeloid cells, monocytes, neutrophils, and eosinophils (Wang, 2009, Immunological Reviews 229: 192-215). CD137 expression has also been demonstrated on tumor vasculature (Broll, 2001, Amer. J. Clin. Pathol. 115(4):543-549; Seaman, 2007, Cancer Cell 11: 539-554) and at sites of inflamed or atherosclerotic endothelium (Drenkard, 2007 FASEB J. 21: 456-463; Olofsson, 2008, Circulation 117: 1292-1301). The ligand that stimulates CD137, i.e., CD137 Ligand (CD137L), is expressed on activated antigen-presenting cells (APCs), myeloid progenitor cells, and hematopoietic stem cells.

Human CD137 is a 255 amino acid protein (GenBank Accession No. NM_001561; NP_001552; SEQ ID NO.: 1). The protein comprises a signal sequence (amino acid residues 1-17), followed by an extracellular domain (169 amino acids), a transmembrane region (27 amino acids), and an intracellular domain (42 amino acids) (Cheuk A T C et al. 2004 Cancer Gene Therapy I1: 215-226). The receptor is expressed on the cell surface in monomer and dimer forms and likely trimerizes with CD137 ligand to signal.

Numerous studies of murine and human T cells indicate that CD137 promotes enhanced cellular proliferation, survival, and cytokine production (Croft, 2009, Nat Rev Immunol 9:271-285). Studies have indicated that some CDI 37 agonist monoclonal antibodies (mAbs) increase costimulatory molecule expression and markedly enhance cytolytic T lymphocyte responses, resulting in anti-tumor efficacy in various models. CD137 agonist mAbs have demonstrated efficacy in prophylactic and therapeutic settings. Further, CD137 monotherapy and combination therapy tumor models have established durable anti-tumor protective T cell memory responses (Lynch, 2008, Immunol Rev. 22: 277-286). CD137 agonists also have been shown to inhibit autoimmune reactions in a variety of art-recognized autoimmunity models (Vinay, 2006, J Mol Med 84:726-736). This dual activity of CD137 offers the potential to provide anti-tumor activity while dampening autoimmune side effects that can be associated with immunotherapy approaches that break immune tolerance.

However, liver-related autoimmune toxicities triggered by agonistic anti-CD137 antibodies have greatly limited their use in clinical applications. There is a long-felt unmet need for antibodies that bind human CD137, increase a CD137-mediated response, and provide potential therapeutics for treatment of various diseases and conditions (e.g., cancer) without immune-mediated side effects.

### BRIEF SUMMARY

In some embodiments, provided herein is a masked antibody comprising a masking peptide (MP) and an antibody that binds human CDI 37, wherein the antibody comprises a heavy chain comprising a heavy chain variable region (VH) and a light chain comprising a light chain variable region (VL); wherein the MP is linked to an N-terminus of the VL, wherein the MP comprises, from N-terminus to C-terminus, a masking unit (MU) and a linkage unit (LU), and wherein the MU comprises an amino acid sequence selected from the group consisting of SEQ ID NOS: 1-7; and wherein the VH comprises a CDR-H1 set forth in TGGVGVG (SEQ ID NO: 36), a CDR-H2 set forth in LIDWADDKYYSPSLKS (SEQ ID NO: 37), and CDR-H3 set forth in GGSDTVIGDWFAY (SEQ ID NO: 38), and wherein the VL comprises a CDR-L1 set forth in RASQSIGSYLA (SEQ ID NO: 39), a CDR-L2 set forth in DASNLET (SEQ ID NO: 40), and a CDR-L3 set forth in QQGYYLWT (SEQ ID NO: 41). In some embodiments, the MP further comprises an N-terminal unit (NU) linked to the N-terminal of the MU. In some embodiments, the N-terminal unit is about 1-10 amino acid residues long. In some embodiments, the N-terminal unit comprises E or EVGSY (SEQ ID NO: 77).

In some embodiments, the LU comprises a first cleavage site. In some embodiments, the first cleavage site is a protease cleavage site for a protease selected from the group consisting of urokinase-type plasminogen activator/uPA, matrix metalloproteinase-1/MMP-1, MMP-2, MMP-3, MMP-8, MMP-9, MMP-14, Tobacco Etch Virus protease/ITEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In some embodiments, the first cleavage site is a protease cleavage site for urokinase-type plasminogen activator/uPA or MMP-9. In some embodiments, the LU further comprises a first linker (L₁) C-terminal to the first cleavage site. In some embodiments, the LU further comprises a second cleavage site. In some embodiments, the second cleavage site is C-terminal to the L₁. In some embodiments, the second cleavage site is a protease cleavage site for a protease selected from the group consisting of urokinase-type plasminogen activator/uPA, matrix metalloproteinase-1/MMP-1, MMP-2, MMP-3, MMP-8, MMP-9, MMP-14, Tobacco Etch Virus protease/TEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In some embodiments, the second cleavage site is a protease cleavage site for urokinase-type plasminogen activator/uPA or MMP-9. In some embodiments, the first and second cleavage sites are the same. In some embodiments, the first and second cleavage sites are different. In some embodiments, the LU further comprises a second linker (L₂) C-terminal to the second cleavage site. In some embodiments, the LU comprises an amino acid sequence selected from the group consisting of SEQ ID NOs: 8-16.

In some embodiments, the masking peptide comprises any one of SEQ ID NOs: 17-35. In some embodiments, the masking peptide comprises SEQ ID NO: 34. In some embodiments, the antibody comprises a VH set forth in SEQ ID NO: 52 and a VL set forth in SEQ ID NO: 53. In some embodiments, the VH comprises SEQ ID NO: 52 and the VL comprises SEQ ID NO: 58.

In some embodiments, the masked antibody is a full length antibody comprising an Fc region. In some embodiments, the Fc region is a human IgG Fc region or a variant thereof. In some embodiments, the human IgG Fc region or variant thereof is a human IgG1, Fc region, a human IgG2 Fc region, a human IgG4 Fc region, or a variant of any one of the preceding. In some embodiments, the masked antibody comprises a variant of a human IgG1 Fc region that comprises substitution(s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D, and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index. In some embodiments, the masked antibody comprises a variant of a human IgG1 Fc region that comprises S267E and L328F substitutions, wherein amino acid numbering is according to the EU index. In some embodiments, the masked antibody comprises an Fc region that comprises SEQ ID NO: 113 or SEQ ID NO: 114.

In some embodiments, the masked antibody comprises the masking peptide set forth in SEQ ID NO: 34, the antibody heavy chain variable domain set forth in SEQ ID NO: 52 and an antibody light chain variable domain set forth in SEQ ID NO: 53, and a human IgG1 Fc region variant comprising S267E and L328F substitutions, wherein amino acid numbering is according to the EU index. In some embodiments, the masked antibody comprises a heavy chain comprising SEQ ID NO: 94 or 95 and a light chain comprising SEQ ID NO: 96. In some embodiments, the masked antibody comprises a variant of a human IgG4 Fc region that comprises substitution(s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index. In some embodiments, the masked antibody comprises a variant of a human IgG4 Fc region that comprises S267E and L328F substitutions, wherein amino acid numbering is according to the EU index. In some embodiments, the masked antibody comprises comprising an Fc region that comprises SEQ ID NO: 117 or 118. In some embodiments, the masked antibody comprises a masking peptide of SEQ ID NO: 34, an antibody heavy chain variable domain set forth in SEQ ID NO: 52 and an antibody light chain variable domain set forth in SEQ ID NO: 53, and a human IgG4 Fc region variant comprising S267E and L328F substitutions, wherein amino acid numbering is according to the EU index. In some embodiments, the masked antibody comprises a heavy chain comprising SEQ ID NO: 92 or 93 and a light chain comprising SEQ ID NO 96.

In some embodiments, the masked antibody is a masked antibody fragment selected from the group consisting of: a Fab, a Fab′, a Fab′-SH, a F(ab′)2, an Fv, an scFv, an (scFv)2, a linear antibody, a single-chain antibody, single domain antibody (nanobody) VHH, a minibody, or a diabody.

In some embodiments, provided are one or more polynucleotides encoding a masked antibody described herein. In some embodiments, provided is a recombinant vector comprising the one or more polynucleotides described herein. In some embodiments, provided is a host cell comprising a vector described herein. In some embodiments, provided is a method of producing a masked antibody, comprising culturing a host cell of claim described herein under appropriate conditions to cause expression of the masked antibody and recovering the masked antibody.

In some embodiments, provided is a method of treating cancer in a subject, comprising administering to the subject an effective amount of a masked antibody provided herein. In some embodiments, cancer is solid tumor cancer. In some embodiments, the solid tumor is breast cancer, liver cancer, colorectal cancer, or colon cancer.

In some embodiments, provided is a method of treating cancer in a subject, comprising administering to the subject an effective amount of a masked antibody described herein and an effective amount of an anti-PD-1 antibody (e.g., an anti-human PD-1 antibody). In some embodiments, provided is a method of treating cancer in a subject, comprising administering to the subject an effective amount of a masked antibody described herein and an effective amount of an anti-CTLA4 antibody (e.g., an anti-human CTLA4 antibody). In some embodiments, provided is a method of treating cancer in a subject, comprising administering to the subject an effective amount of a masked antibody described herein and an effective amount of a bispecific antibody that binds HER2 (e.g., human HER2) and CD3 (e.g., human CD3). In some embodiments, provided is a method of treating cancer in a subject, comprising administering to the subject an effective amount of the masked antibody described herein and effective amount of a bispecific T cell engager (TCE) that targets CD3 (e.g., human CD3) and an antigen expressed on the surface of a solid tumor cancer cell. In some embodiments, the antigen expressed on the surface of the solid tumor is HER2 (e.g., human HER2). In some embodiments, the TCE that targets CD3 and HER2 comprises three polypeptide chains, wherein a first polypeptide chain comprises an antibody heavy chain, a second polypeptide chain comprises an antibody light chain, and a third polypeptide chain comprises an scFv-Fc domain fusion wherein the C-terminus of the ScFv is fused to the N-terminus of the Fc domain, wherein the first and second polypeptide chains associate to from a HER-2 binding arm, wherein the third polypeptide chain binds CD3, and wherein the Fc domain of the anti-HER2 binding arm and the Fc of the third polypeptide chain dimerize to form the TCE. In some embodiments, the TCE is a masked TCE (e.g., wherein the portion of the TCE that binds CD3 and/or the portion of the TCE that binds HER2 are masked). In some embodiments, the first polypeptide chain comprises SEQ ID NO: 125, the second polypeptide chain comprises SEQ ID NO: 124, and the third polypeptide chain comprises SEQ ID NO: 126. In some embodiments, the cancer is solid tumor. In some embodiments, the solid tumor is colon cancer, breast cancer, liver cancer, colorectal cancer, or colon cancer.

In some embodiments, provided is a kit comprising a masked antibody described for treating an individual having cancer according to a method described herein. In some embodiments, provided is a kit comprising a masked antibody described herein for use in combination with an anti-PD-1 antibody (e.g., an anti-human PD-1 antibody) for treating an individual having cancer according to a method described herein. In some embodiments, provided is a kit comprising a masked antibody described herein for use in combination with an anti-CTLA4 antibody (e.g., an anti-human CTLA4 antibody) for treating an individual having cancer according to a method described herein. In some embodiments, provided is a kit comprising a masked antibody described herein for use in combination with a bispecific antibody that binds HER2 (e.g., human HER2) and CD3 (e.g., human CD3) for treating an individual having cancer according to a method described herein. In some embodiments, provided is a kit comprising a masked antibody described herein for use in combination with bispecific T cell engager (TCE) that targets CD3 (e.g., human CD3) and an antigen expressed on the surface of a solid tumor cancer cell for treating an individual having cancer according to a method described herein. In some embodiments, the antigen expressed on the surface of the solid tumor is HER2 (e.g., human HER2). In some embodiments, the TCE that targets CD3 and HER2 comprises three polypeptide chains, wherein a first polypeptide chain comprises an antibody heavy chain, a second polypeptide chain comprises an antibody light chain, and a third polypeptide chain comprises an scFv-Fc domain fusion wherein the C-terminus of the ScFv is fused to the N-terminus of the Fc domain, wherein the first and second polypeptide chains associate to from a HER-2 binding arm, wherein the third polypeptide chain binds CD3, and wherein the Fc domain of the anti-HER2 binding arm and the Fc of the third polypeptide chain dimerize to form the TCE. In some embodiments, the TCE is a masked TCE (e.g., wherein the portion of the TCE that binds CD3 and/or the portion of the TCE that binds HER2 are masked). In some embodiments, the first polypeptide chain comprises SEQ ID NO: 125, the second polypeptide chain comprises SEQ ID NO: 124, and the third polypeptide chain comprises SEQ ID NO: 126. It is to be understood that one, some, or all of the properties of the various embodiments described above and herein may be combined to form other embodiments of the present disclosure. These and other aspects of the present disclosure will become apparent to one of skill in the art. These and other embodiments of the present disclosure are further described by the detailed description that follows.

### BRIEF DESCRIPTIONS OF THE DRAWINGS

FIG. 1A shows the results of FACS-based assays that were performed to assess the masking efficiency of exemplary CD137 masked antibodies, as compared to parental antibodies TY21242 and TY23310, against human CD137 displayed on the surfaces of yeast cells. FIG. 1B also shows the results of FACS-based assays that were performed to assess the masking efficiency of exemplary CDI 37 masked antibodies, as compared to parental antibodies TY21242 and TY23310, against human CD137 displayed on the surfaces of yeast cells. FIG. 1C shows additional results of FACS-based assays that were performed to assess the masking efficiency of exemplary CD137 masked antibodies, as compared to parental antibodies TY21242 and TY23310, against human CD137 displayed on the surfaces of yeast cells. FIG. 1D shows further results of FACS-based assays that were performed to assess the masking efficiency of exemplary CD137 masked antibodies, as compared to parental antibodies TY21242 and TY23310, against human CD137 displayed on the surfaces of yeast cells.

FIG. 2A shows the results of ELISA assays that were performed to assess the activities of masked anti-CD137 antibodies TY25366 and TY25368, as compared to parental antibodies TY24118 and TY24122, respectively, before removal of the masking peptide (i.e., without MMP9 treatment) and after removal of the masking peptide (i.e., with MMP9 treatment). See also Table D.

FIG. 2B shows the results of FACS-based assays that were performed to assess the activities of masked anti-CD137 antibodies TY25366 and TY25368, as compared to parental antibodies TY24118 and TY24122, respectively, before removal of the masking peptide (i.e., without MMP9 treatment) and after removal of the masking peptide (i.e., with MMP9 treatment). See also Table D.

FIGS. 3A-3E show size-exclusion chromatography (SEC) profiles of exemplary masked antibodies TY25366 and TY25368 under accelerated stress conditions. FIG. 3A(i) shows the SEC profiles of TY25366 after 0, 3, and 6 cycles of freezing and thawing. FIG. 3A(ii) shows the SEC profiles of TY25368 after 0, 3, and 6 cycles of freezing and thawing. FIG. 3B(i) shows the SEC profiles of TY25366 after 0, 7, 14, 21, and 28 days of storage at 40° C. FIG. 3B(ii) shows the SEC profiles of TY25368 after 0, 7, 14, 21, and 28 days of storage at 40° C. FIG. 3C(i) shows the SEC profiles of TY25366 after storage in acidic buffer (sodium acetate solution, pH3.6) after 0 and 2 hours at room temperature. FIG. 3C(ii) shows the SEC profiles of TY25368 after storage the same acidic buffer after 0 and 2 hours at room temperature. FIG. 3D(i) shows the SEC profiles of TY25366 after storage in 50 mM Histidine, 300 mM NaCl, pH7.0 buffer after 0 and 24 h at 40° C. FIG. 3D(ii) shows the SEC profiles of TY25368 after storage in 50 mM Histidine, 300 mM NaCl, pH7.0 buffer after 0 and 24 h at 40° C. FIG. 3E(i) shows the SEC profiles of TY25366 and TY25368 before and after storage in saline for 6 h at room temperature 24 h at 4° C., as compared to the control condition.

FIG. 4A(i) shows the results of FACS-based assays that were performed to assess the binding of binding of MMP9-treated TY25368 and untreated TY25368 to naïve and activated CD4⁺ human T cells. FIG. 4A(ii) shows the results of FACS-based assays that were performed to assess the binding of binding of MMP9-treated TY25368 and untreated TY25368 to naïve and activated CD8′ human T cells. FIG. 4B(i) shows the results of FACS-based assays that were performed to assess the binding of binding of MMP9-treated TY25368 and untreated TY25368 to naïve and activated CD4⁺ cynomolgus T cells. FIG. 4B(ii) shows the results of FACS-based assays that were performed to assess the binding of binding of MMP9-treated TY25368 and untreated TY25368 to naïve and activated CD8⁺ cynomolgus T cells. FIG. 4C(i) shows the results of FACS-based assays that were performed to assess the binding of binding of MMP9-treated TY25368 and untreated TY25368 to naïve and activated CD4⁺ mouse T cells. FIG. 4C(i) shows the results of FACS-based assays that were performed to assess the binding of binding of MMP9-treated TY25368 and untreated TY25368 to naïve and activated CD8′ mouse T cells. FIG. 4D(i) shows the results of FACS-based assays that were performed to assess the binding of binding of MMP9-treated TY25368 and untreated TY25368 to naïve and activated CD4⁺ rat T cells. FIG. 4D(i) shows the results of FACS-based assays that were performed to assess the binding of binding of MMP9-treated TY25368 and untreated TY25368 to naïve and activated CD8⁺ rat T cells.

FIG. 5 shows the results of ELISA experiments that were performed to assess the degree to which MMP9-treated and untreated TY25368 blocks the binding of CD137 to its ligand.

FIG. 6A shows the results of experiments that were performed to assess the degree to which different masked and non-masked anti-CD137 antibodies stimulate CD137 signaling. CD137-mediated signaling was determined using a CD137 reporter gene assay in the presence of CHO-K1-hFcgRIIb cells as cross-linker (E:CL=20:1). FIG. 6B shows the results of experiments that were performed to assess the degree to which different masked and non-masked anti-CD137 antibodies stimulate CD137 signaling. CD137-mediated signaling was determined using a CD137 reporter gene assay in the absence of CHO-K1-hFcgRIIb cells as cross-linker.

FIG. 7A shows the results of experiments that were performed to assess the degree wo which different masked and non-masked anti-CD137 antibodies stimulate CD137 signaling. CD137-mediated signaling was determined using a CDI 37 reporter gene assay in the presence of human primary B cells as cross-linker (E:CL=5:1). FIG. 7B shows the results of experiments that were performed to assess the degree wo which different masked and non-masked anti-CD137 antibodies stimulate CD137 signaling. CD137-mediated signaling was determined using a CD137 reporter gene assay in the presence of human primary B cells as cross-linker (E:CL=20:1).

FIG. 8A shows the results of ELISA-based assays that were performed to determine whether anti-CD137 antibodies enhance SEA-stimulated cytokine secretion by human PBMCs obtained from Donor #102. FIG. 8B shows the results of ELISA-based assays that were performed to determine whether anti-CD137 antibodies enhance SEA-stimulated cytokine secretion by human PBMCs obtained from Donor #142.

FIG. 9 shows the results of experiments that were performed to assess ADCC activity of an anti-CD137 antibodies and a masked anti-CD137 antibody.

FIG. 10 shows the results of experiments that were performed to assess CDC activity of a masked anti-CD137 antibody.

FIG. 11 provides the results of in vivo experiments that were performed assess the efficacy of (a) vehicle, (b) masked anti-CD137 antibody TY25368, (c) anti-PD-1 antibody FG1225, and (d) TY25368 in combination with FG1225 in inhibiting the growth of CT26 murine colon tumors in a mouse allograft model.

FIG. 12 provides the results of in vivo experiments that were performed assess the efficacy of (a) vehicle, (b) masked anti-CD137 antibody TY25368, (c) masked anti-CTLA4 antibody TY21580, (d) masked anti-CTLA4 antibody TY22404, (e) masked anti-CD137 antibody TY25368 and masked anti-CTLA4 antibody TY21580, and (f) masked anti-CD137 antibody TY25368 and masked anti-CTLA4 antibody TY22404 in inhibiting the growth of MC28 murine colon tumors in a mouse allograft model.

FIG. 13 provides a concentration-time profile for intact and total forms of TY25366 and TY25368 at 30 and 100 mg/kg doses in cynomolgus monkeys.

FIG. 14A shows the results of experiments that were performed to assess the activation of CD137-mediated cell signaling by anti-CD137 antibodies comprising Fc mutation(s) in the presence of CHO-K1-hFcγRIIb as cross-linker. FIG. 14B shows the results of experiments that were performed to assess the activation of CD137-mediated cell signaling by anti-CD137 antibodies comprising Fc mutation(s) in the presence of CHO-K1-mFcγRIIb as cross-linker. FIG. 14C shows the results of experiments that were performed to assess the activation of CD137-mediated cell signaling by anti-CD137 antibodies comprising Fc mutation(s) in the absence of cross-linker.

FIG. 15A shows the results of experiments that were performed to assess the degree to which anti-CD137 antibodies comprising Fc mutation(s) that have been immobilized on a solid support enhance SEA-stimulated cytokine secretion by human PBMCs. FIG. 15B shows the results of experiments that were performed to assess the degree to which soluble anti-CD137 antibodies comprising Fc mutation(s) enhance SEA-stimulated cytokine secretion by human PBMCs.

FIG. 16 provides the results of in vivo experiments that were performed assess the efficacy of (a) vehicle, (b) 3 mg/kg masked anti-CD137 antibody TY25368, (c) 1 mg/kg masked anti-CD137 antibody TY25368, and (d) 0.3 mg/kg masked anti-CD137 antibody TY25368 in inhibiting the growth of EMT6 murine breast cancer tumors in a mouse allograft model.

FIG. 17 provides the results of in vivo experiments that were performed assess the efficacy of (a) vehicle, (b) 5 mg/kg TY24118, (c) 5 mg/kg TY24122, (d) 5 mg/kg TY25366, or (e) 5 mg/kg TY25368 in inhibiting the growth of H22 murine liver cancer tumors in a mouse allograft model.

FIG. 18A provides the results of in vivo experiments that were performed assess the efficacy of (a) vehicle, (b) 5 mg/kg TY24118, (c) 5 mg/kg TY24122, (d) 5 mg/kg TY25366, or (e) 5 mg/kg TY25368 in inhibiting the growth of CT26 murine colorectal cancer tumors in a mouse allograft model. FIG. 18B provides the results of in vivo experiments that were performed assess the efficacy of (a) vehicle, (b) 1 mg/kg TY24118, (c) 1 mg/kg TY24122, (d) 1 mg/kg TY25366, or (e) 1 mg/kg TY25368 in inhibiting the growth of CT26 murine colorectal cancer tumors in a mouse allograft model.

FIG. 19A provides the results of in vivo experiments that were performed assess the efficacy of (a) vehicle, (b) 5 mg/kg TY25368, (c) 5 mg/kg TY27151, or (d) 5 mg/kg TY25368 and 5 mg/kg TY27151 in inhibiting the growth of MC38 murine colon cancer tumors expressing human HER2 and human B7H3 in a mouse syngeneic tumor model. FIG. 19B provides tumor growth curves of individual mice in each of treatment groups (a), (b), (c), and (d).

FIG. 20 provides a schematic depiction of TY27151

### DETAILED DESCRIPTION

### Overview

The toxicity arising from generalized stimulation of T cells restricts applicability of many CD137 agonists in cancer immune therapy. The present disclosure provides masked anti-CD137 antibodies that are effective in the treatment of cancer and without causing significant safety issues.

### Definitions

Before describing the present disclosure in detail, it is to be understood that this present disclosure is not limited to particular compositions or biological systems, which can, of course, vary. It is also to be understood that the terminology used herein is for the purpose of describing particular embodiments only and is not intended to be limiting.

As used herein, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure described herein include “comprising,” “consisting,” and “consisting essentially of” aspects and embodiments.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “polypeptide,” “protein,” and “peptide” are used interchangeably herein and may refer to polymers of two or more amino acids.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “isolated nucleic acid” refers to a nucleic acid molecule of genomic, cDNA, or synthetic origin, or a combination thereof, which is separated from other nucleic acid molecules present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid of interest.

The term “antibody” is used herein in the broadest sense and specifically covers monoclonal antibodies (including full length monoclonal antibodies), polyclonal antibodies, masked antibodies (e.g., activatable or non-activatable antibodies), multispecific antibodies (e.g., bispecific antibodies), and antibody fragments (e.g., a single-chain variable fragment or scFv) so long as they exhibit the desired biological activity (e.g., the ability to bind a target antigen with desired specificity and affinity).

In some embodiments, the term “antibody” refers to an antigen-binding protein (i.e., immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as V_{H}) followed by a constant region. The heavy chain constant region is comprised of three domains, C_{H1}, C_{H2} and C_{H3}. Each light chain has, at the N-terminus, a variable region (abbreviated herein as V_{I}) followed by a constant region at its other end. The light chain constant region is comprised of one domain, C_{L}. The V_{L} is aligned with the V_{H} and the C_{L} is aligned with the first constant domain of the heavy chain (CH1). The pairing of a V_{H} and V_{L} together forms a single antigen-binding site. An IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain.

The V_{H} and V_{L} regions can be further subdivided into regions of hypervariability, termed hyper-variable regions (HVR) based on structural and sequence analysis. HVRs are interspersed with regions that are more conserved, termed framework regions (FW) (see e.g., Chen et al. (1999) J. Mol. Biol. (1999) 293, 865-881). Each V_{H} and V_{L} is composed of three HVRs and four FWs, arranged from amino-terminus to carboxy-terminus in the following order: FW-1_HVR-1_FW-2_HVR-2_FW-3_HVR-3_FW4. Throughout the present disclosure, the three HVRs of the heavy chain are referred to as HVR-H1, HVR-H2, and HVR-H3. Similarly, the three HVRs of the light chain are referred to as HVR-L1, HVR-L2, and HVR-L3.

Table 1 below provides exemplary CDR definitions according to various algorithms known in the art.

TABLE 1
CDR Definitions

| | Kabat¹ | Chothia² | MacCallum³ | IMGT⁴ | AHo⁵ |
|---|---|---|---|---|---|
| CDR-H1 | 31-35 | 26-32 | 30-35 | 27-38 | 25-40 |
| CDR-H2 | 50-65 | 53-55 | 47-58 | 56-65 | 58-77 |
| CDR-H3 | 95-102 | 96-101 | 93-101 | 105-117 | 109-137 |
| CDR-L1 | 24-34 | 26-32 | 30-36 | 27-38 | 25-40 |
| CDR-L2 | 50-56 | 50-52 | 46-55 | 56-65 | 58-77 |
| CDR-L3 | 89-97 | 91-96 | 89-96 | 105-117 | 109-137 |

¹Residue numbering follows the nomenclature of Kabat et al., J. Biol. Chem. 252: 6609-6616 (1977); Kabat et al., U.S. Dept. of Health and Human Services, “Sequences of proteins of immunological interest” (1991).
²Residue numbering follows the nomenclature of Chothia et al., J. Mol. Biol. 196: 901-917 (1987); Al-Lazikani B. et al., J. Mol. Biol., 273: 927-948 (1997).
³Residue numbering follows the nomenclature of MacCallum et al., J. Mol. Biol. 262: 732-745 (1996); Abhinandan and Martin, Mol. Immunol., 45: 3832-3839 (2008).
⁴Residue numbering follows the nomenclature of Lefranc M. P. et al., Dev. Comp. Immunol., 27: 55-77 (2003); and Honegger and Pluckthun, J. Mol. Biol., 309:657-670 (2001).
⁵Residue numbering follows the nomenclature of Honegger and Pluckthun, J. Mol. Biol., 309:657-670 (2001).

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Within light and heavy chains, the variable and constant regions are joined by a “J” region of about 12 or more amino acids, with the heavy chain also including a “D” region of about 10 or more amino acids (see e.g., Fundamental Immunology Ch. 7 (Paul, W., ed., 2^{nd} ed. Raven Press, N.Y). (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. There are five classes of antibodies: IgA, IgD, IgE, IgG, and IgM, having heavy chains designated α (alpha), δ (delta), ε (epsilon), γ (gamma), and μ (mu), respectively. The IgG class of antibody can be further classified into four subclasses IgG1, IgG2, IgG3, and IgG4 by the gamma heavy chains, Y1-Y4, respectively.

The term “antigen-binding fragment,” “antigen binding portion” or “antigen binding domain” of an antibody, used herein interchangeably, refers to one or more portions of an antibody that retain the ability to bind to the antigen that the antibody bonds to. Examples of “antigen-binding fragments” of an antibody include, but are not limited to, (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Other exemplary antigen-binding fragments are described elsewhere herein.

The term “masked antibody” refers to an antibody, or an antigen-binding fragment thereof, comprising a masking peptide that interferes with, obstructs, reduces the ability of, prevents, inhibits, or competes with the antigen binding domain of the antibody, for binding to its target. A masked antibody may be generated by linking a masking peptide to the antigen-binding domain of an antibody. In some embodiments, a masked antibody, or an antigen-binding fragment thereof, exhibits a first binding affinity to a target when in an inactivated state (e.g., inhibited or masked by a masking peptide), and exhibits a second binding affinity to the target in an activated state (e.g., uninhibited or unmasked by the masking peptide (e.g., the masking peptide is cleaved from the antibody)), where the second binding affinity is greater than the first binding affinity. A masked antibody may be generated by linking a masking peptide comprising an activatable component (e.g., a cleavable site within a linkage unit, or “LU”) to the antigen binding domain of an antibody.

A “masking peptide” refers to a peptide which inhibits binding of an antigen binding domain to its target antigen, and typically comprises, from N terminus to C terminus, a masking unit (MU) and a linkage unit (LU). The C terminus of the masking peptide is typically linked to the N terminus of the VH or the VL of the antigen-binding domain. In some embodiments, the masking peptide, or a portion thereof, interferes with or inhibits binding of the antigen binding domain to its target so efficiently that binding of the antigen-binding domain to its target is extremely low and/or below the limit of detection (e.g., binding cannot be detected in an ELISA or flow cytometry assay). The masked antibodies or polypeptides described herein may comprise one or more linkers, e.g., within the LU, disposed between MU and LU, LU and VH or VL, or VH and hinge region of an Fc.

The LU of the masking peptide may comprise at least one cleavable site. A cleavage site generally includes an amino acid sequence that is cleavable, for example, serves as the substrate for an enzyme and/or a cysteine-cysteine pair capable of forming a reducible disulfide bond. As such, when the terms “cleavage,” “cleavable,” “cleaved” and the like are used in connection with a cleavage site, the terms encompass enzymatic cleavage, e.g., by a protease, as well as disruption of a disulfide bond between a cysteine-cysteine pair via reduction of the disulfide bond that can result from exposure to a reducing agent. The amino acid sequence of the cleavage site may overlap with or be included within the MU. Masked antibodies or masked polypeptides may comprise a cleavage site configured to mediate activation of the antibody or the polypeptide. For example, when the cleavage site of an activatable antibody is intact (e.g., uncleaved by a corresponding enzyme, and/or containing an unreduced cysteine-cysteine disulfide bond), the masking peptide, or a portion thereof, may interfere with or inhibit binding of the antigen binding domain to its target. In some embodiments, the LU of the masking peptide does not comprise a cleavable site.

The term “masking efficiency” refers to the efficiency with which the masking peptide inhibits binding of the antigen binding domain to the target antigen. Masking efficiency may be measured as the difference in or the ratio of the binding affinity of a masked antibody or masked polypeptide comprising an antigen binding domain and the binding affinity of an unmasked antibody or unmasked polypeptide comprising an antigen binding domain (e.g., the masking peptide is cleaved from the antibody). For example, the masking efficiency may be measured by dividing the EC50 or K_{D} of a masked antibody for binding a target antigen in its inactivated (e.g., inhibited, masked, and/or uncleaved) state, relative to the EC50 or K_{D} of the unmasked antibody to bind to the target antigen in its activated (e.g., uninhibited, unmasked, and/or cleaved) state, or relative to EC50 or K_{D} of the parental antibody (e.g., not linked to a masking peptide) to bind to the target antigen. The EC50 values may be measured in an ELISA assay, or a Jurkat NFAT reporter assay, for example, as described in U.S. Pat. App. Pub. No. US20210207126 A1. The K_{D} values may be measured by, for example, using surface plasmon resonance.

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants (see e.g., Epitope Mapping Protocols in Methods in Molecular Biology, Vol. 66, G. E. Morris, Ed. (1996)). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

The term “germline” refers to the nucleotide sequences of the antibody genes and gene segments as they are passed from parents to offspring via the germ cells. The germline sequence is distinguished from the nucleotide sequences encoding antibodies in mature B cells which have been altered by recombination and hypermutation events during the course of B cell maturation.

The term “glycosylation sites” refers to amino acid residues which are recognized by a eukaryotic cell as locations for the attachment of sugar residues. The amino acids where carbohydrate, such as oligosaccharide, is attached are typically asparagine (N-linkage), serine (0-linkage), and threonine (0-linkage) residues. The specific site of attachment is typically signaled by a sequence of amino acids, referred to herein as a “glycosylation site sequence”. The glycosylation site sequence for N-linked glycosylation is: -Asn-X-Ser- or -Asn-X-Thr-, where X may be any of the conventional amino acids, other than proline. The terms “N-linked” and “O-linked” refer to the chemical group that serves as the attachment site between the sugar molecule and the amino acid residue. N-linked sugars are attached through an amino group; O-linked sugars are attached through a hydroxyl group. The term “glycan occupancy” refers to the existence of a carbohydrate moiety linked to a glycosylation site (i.e., the glycan site is occupied). Where there are at least two potential glycosylation sites on a polypeptide, either none (0-glycan site occupancy), one (1-glycan site occupancy) or both (2-glycan site occupancy) sites can be occupied by a carbohydrate moiety.

The term “host cell” refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; human cells (e.g., HEK293F cells, HEK293T cells; or human tissues or hybridoma cells, yeast cells, insect cells (e.g., S2 cells), bacterial cells (e.g., E. coli cells) and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell but are still included within the scope of the term “host cell.”

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “humanized antibody” refers to a chimeric antibody that contains amino acid residues derived from human antibody sequences. A humanized antibody may contain some or all of the CDRs or HVRs from a non-human animal or synthetic antibody while the framework and constant regions of the antibody contain amino acid residues derived from human antibody sequences.

The term “exemplary antibody” refers to any one of the antibodies described herein. These antibodies may be in any class (e.g., IgA, IgD, IgE, IgG, and IgM). Thus, each antibody identified above encompasses antibodies in all five classes that have the same amino acid sequences for the V_{L} and V_{H} regions. Further, the antibodies in the IgG class may be in any subclass (e.g., IgG1 IgG2, IgG3, and IgG4). Thus, each antibody identified above in the IgG subclass encompasses antibodies in all four subclasses that have the same amino acid sequences for the V_{L} and V_{H} regions. The amino acid sequences of the heavy chain constant regions of human antibodies in the five classes, as well as in the four IgG subclasses, are known in the art. The amino acid sequence of the full length heavy chain and light chain for the IgG4 subclass of each of the illustrative antibodies shown in in Table 1b is provided in the disclosure.

An “isolated” antibody or binding molecule is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see e.g., Flatman et al., J. Chromatogr. B 848:79-87 (2007).

The term “kₐ” refers to the association rate constant of a particular antibody-antigen interaction, whereas the term “k_{d}” refers to the dissociation rate constant of a particular antibody-antigen interaction.

The term “K_{D}” refers to the equilibrium dissociation constant of a particular antibody-antigen interaction. It is obtained from the ratio of k_{d} to kₐ (i.e., k_{d}/kₐ) and is expressed as a molar concentration (M). K_{D} is used as a measure for the affinity of an antibody's binding to its binding partner. The smaller the K_{D}, the more tightly bound the antibody is, or the higher the affinity between antibody and the antigen. For example, an antibody with a nanomolar (nM) dissociation constant binds more tightly to a particular antigen than an antibody with a micromolar (μM) dissociation constant. K_{D} values for antibodies can be determined using methods well established in the art. One method for determining the K_{D} of an antibody is by using an ELISA. For example, an assay procedure using an ELISA.

The term “mammal” refers to any animal species of the Mammalia class. Examples of mammals include: humans; laboratory animals such as rats, mice, hamsters, rabbits, non-human primates, and guinea pigs; domestic animals such as cats, dogs, cattle, sheep, goats, horses, and pigs; and captive wild animals such as lions, tigers, elephants, and the like.

The term “prevent” or “preventing,” with reference to a certain disease condition in a mammal, refers to preventing or delaying the onset of the disease, or preventing the manifestation of clinical or subclinical symptoms thereof.

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see e.g., Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucleic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

As used herein, the term “binds”, “binds to”, “specifically binds” “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. For example, a masked anti-CD137 antibody described herein is said to selectively bind to human CD137 if it binds to human CD137 at an EC50 that is below 10 percent of the EC50 at which it binds to different antigen in an in vitro assay.

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement, such as sonograms, nuclear magnetic resonance testing and positron emissions testing.

The term “vector” refers to a nucleic acid molecule capable of transporting a foreign nucleic acid molecule. The foreign nucleic acid molecule is linked to the vector nucleic acid molecule by a recombinant technique, such as ligation or recombination. This allows the foreign nucleic acid molecule to be multiplied, selected, further manipulated, and/or expressed in a host cell or organism. A vector can be a plasmid, phage, transposon, cosmid, chromosome, virus, or virion. One type of vectors can be integrated into the genome of a host cell upon introduction into the host cell, and thereby are replicated along with the host genome (e.g., non-episomal mammalian vectors). Another type of vector is capable of autonomous replication in a host cell into which it is introduced (e.g., bacterial vectors having a bacterial origin of replication and episomal mammalian vectors). Another specific type of vector capable of directing the expression of expressible foreign nucleic acids to which they are operatively linked is commonly referred to as “expression vectors.” Expression vectors generally have control sequences that drive expression of the expressible foreign nucleic acids. Simpler vectors, known as “transcription vectors,” are only capable of being transcribed but not translated: they can be replicated in a target cell but not expressed. The term “vector” encompasses all types of vectors regardless of their function. Vectors capable of directing the expression of expressible nucleic acids to which they are operatively linked are commonly referred to “expression vectors.” Other examples of “vectors” may include display vectors (e.g., vectors that direct expression and display of an encoded polypeptide on the surface of a virus or cell (such as a bacterial cell, yeast cell, insect cell, and/or mammalian cell).

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

An “effective amount” refers to at least an amount effective, at dosages and for periods of time necessary, to achieve one or more desired or indicated effects, including a therapeutic or prophylactic result. An effective amount can be provided in one or more administrations. For purposes of the present disclosure, an effective amount of antibody, drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition (e.g., an effective amount as administered as a monotherapy or combination therapy). Thus, an “effective amount” may be considered in the context of administering one or more therapeutic agents, and a single agent may be considered to be given in an effective amount if, in conjunction with one or more other agents, a desirable result may be or is achieved.

The methods and techniques of the present disclosure are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990). Enzymatic reactions and purification techniques are performed according to manufacturer's specifications, as commonly accomplished in the art, or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the twenty conventional amino acids and their abbreviations follow conventional usage. See Immunology—A Synthesis (2nd Edition, E. S. Golub, and D. R. Gren, Eds., Sinauer Associates, Sunderland, Mass. (1991)).

All references cited herein, including patent applications and publications, are hereby incorporated by reference in their entirety.

### Masked Anti-CD137 Antibodies

In one aspect, provided is a masked antibody comprising a masking peptide (MP) and an antibody that binds human CD137 (hCD137), wherein the antibody comprises a heavy chain variable region (VH) and a light chain variable region (VL), wherein the MP is linked to an N-terminus of the VL, wherein the MP comprises, from N-terminus to C-terminus, a masking unit (MU), a linkage unit (LU), wherein the MU comprises an amino acid sequence selected from the group consisting of SEQ ID NOs: 1-7, and wherein the VH comprises a CDR-H1 set forth in TGGVGVG (SEQ ID NO: 36), a CDR-H2 set forth in LIDWADDKYYSPSLKS (SEQ ID NO: 37), and CDR-H3 set forth in GGSDTVIGDWFAY (SEQ ID NO: 38), and wherein the VL comprises a CDR-L1 set forth in RASQSIGSYLA (SEQ ID NO: 39), a CDR-L2 set forth in DASNLET (SEQ ID NO: 40), and a CDR-L3 set forth in QQGYYLWT (SEQ ID NO: 41). In some embodiments, provided is a masked antibody comprising a masking peptide (MP) and an antibody that binds human CD137 (hCD137), wherein the antibody comprises a heavy chain variable region (VH) and a light chain variable region (VL), wherein the MP is linked to an N-terminus of the VL, wherein the MP comprises, from N-terminus to C-terminus, a masking unit (MU), a linkage unit (LU), and wherein the VH comprises a CDR-H1 set forth in TSGVGVG (SEQ ID NO: 42), a CDR-H2 set forth in LIDWDDDKYYSPSLKS (SEQ ID NO: 43), and CDR-H3 set forth in GGSDTVLGDWFAY (SEQ ID NO: 44), and wherein the VL comprises a CDR-L1 set forth in RASQSVSPYLA (SEQ ID NO: 45), a CDR-L2 set forth in DASSLES (SEQ ID NO: 46), and a CDR-L3 set forth in QQGYSLWT (SEQ ID NO: 47). In some embodiments, provided is a masked antibody comprising a masking peptide (MP) and an antibody that binds human CDI 37 (hCD137), wherein the antibody comprises a heavy chain variable region (VH) and a light chain variable region (VL), wherein the MP is linked to an N-terminus of the VL, wherein the MP comprises, from N-terminus to C-terminus, a masking unit (MU), a linkage unit (LU), and wherein the VH comprises a CDR-H1 set forth in SGHYWA (SEQ ID NO: 48), a CDR-H2 set forth in SISGYGSTTYYADSVKG (SEQ ID NO: 49), and CDR-H3 set forth in GGSDAVLGDWFAY (SEQ ID NO: 50), and wherein the VL comprises a CDR-L1 set forth in RASQGIGSFLA (SEQ ID NO: 51), a CDR-L2 set forth in DASNLET (SEQ ID NO: 40), and a CDR-L3 set forth in QQGYYLWT (SEQ ID NO: 41). SEQ ID NOs: 1-7 are provided in Table 2 below:

TABLE 2
Exemplary Masking Unit Sequences
(SEQ ID NOs: 1-7)

| SEQ ID NO: | MASKING UNIT (MU) SEQUENCES |
|---|---|
| 1 | PTDLDACADAPNHCHF |
| 2 | SSTHAHCHHSPANCIS |
| 3 | DTDYDFCPILRHRCDS |
| 4 | NDYNYHCKWRPSRCHN |
| 5 | YHDYDDCRVLPRRCFN |
| 6 | SNNFASCLWRHRSCAD |
| 7 | TDNYDYCPRLRRKCYH |

In some embodiments, the MP further comprises a N-terminal unit. In some embodiments, the N-terminal unit is between about 1 and 10 amino acids in length. In some embodiments, the N-terminal unit comprises E (glutamic acid) or EVGSY (SEQ ID NO: 77). In some embodiments, the LU comprises at least a first cleavage site (CS₁) (e.g., a first protease cleavage site). In some embodiments, the LU further comprises a second cleavage site (CS₂). In some embodiments, the first and/or second cleavage site are a protease cleavage site. In some embodiments, the first and second cleavage sites are the same. In some embodiments, the first and second cleavage sites are different. Any suitable protease cleavage site recognized and/or cleaved by any protease (e.g., a protease that is known to be co-localized with a target of a polypeptide comprising the cleavage site) known in the art may be used, including, for example, a protease cleavage site recognized and/or cleaved by urokinase-type plasminogen activator (uPA); matrix metalloproteinases (e.g., MMP-1, MMP-2, MMP-3, MMP-7, MMP-8, MMP-9, MMP-10, MMP-11, MMP-12, MMP-13, MMP-14, MMP-15, MMP-16, MMP-17, MMP-19, MMP-20, MMP-23, MMP-24, MMP-26, and/or MMP-27); Tobacco Etch Virus (TEV) protease; plasmin; Thrombin; PSA; PSMA; ADAMS/ADAMTS (e.g., ADAM 8, ADAM 9, ADAM10, ADAM12, ADAM15, ADAM17/TACE, ADAMDEC1, ADAMTS1, ADAMTS4, and/or ADAMTS5); caspases (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, and/or Caspase-14); aspartate proteases (e.g., RACE and/or Renin); aspartic cathepsins (e.g., Cathepsin D and/or Cathepsin E); cysteine cathepsins (e.g., Cathepsin B, Cathepsin C, Cathepsin K, Cathepsin L, Cathepsin S, Cathepsin V/L2, and/or Cathepsin X/Z/P); cysteine proteinases (e.g., Cruzipain, Legumain, and/or Otubain-2); KLKs (e.g., KLK4, KLK5, KLK6, KLK7, KLK8, KLK10, KLK 11, KLK13, and/or KLK14); metallo proteainases (e.g., Meprin, Neprilysin, PSMA, and/or BMP-1); serine proteases (e.g., activated protein C. Cathepsin A, Cathepsin G, Chymase, and/or coagulation factor proteases (such as FVIIa, FIXa, FXa, FXIa, FXIIa)); elastase; granzyme B; guanidinobenzoatase; HtrA1; human neutrophil elastase; lactoferrin; marapsin; NS3/4A; PACE4; tPA; tryptase; type II transmembrane serine proteases (TTSPs) (e.g., DESC1, DPP-4, FAP, Hepsin, Matriptase-2, MT-SP1/Matriptase, TMPRSS2, TMPRSS3 and/or TMPRSS4); etc. In some embodiments, the first protease cleavage site is a cleavage site for a protease selected from uPA, MMP-1, MMP-2, MMP-3, MMP-8, MMP-9, MMP-14, TEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. In some embodiments, the first protease cleavage site is a cleavage site for a protease selected from uPA, MMP-2, MMP-9, and/or TEV protease. In some embodiments, the protease cleavage comprises an amino acid sequence selected from SGRSA (SEQ ID NO: 86) and PLGLAG (SEQ ID NO: 87).

In some embodiments, the LU further comprises a first linker (L₁). In some embodiments, the first linker (L₁) is C-terminal to the first cleavage site (CS₁) (e.g., a first protease cleavage site). In some embodiments, the LU comprises a structure, from N-terminus to C-terminus, of: (CS₁)-L₁. In some embodiments, the LU further comprises a second linker (L₂). In some embodiments, the L₂ is C-terminal to the second cleavage site. In some embodiments, the LU comprises a structure, from N-terminus to C-terminus, of: (CS₁)-L₁-(CS₂)-L₂. In some embodiments, L₁ and L₂ are any suitable linker (e.g., a flexible linker) known in the art, including, without limitation, e.g., glycine polymers (G)n, where n is an integer of at least 1 (e.g., at least one, at least 2, at least 3, at least 4, at least 5, at least 6, at least 7, at least 8, at least 9, at least 10, etc.); glycine-serine polymers (GS)n, where n is an integer of at least 1 (e.g., at least one, at least 2, at least 3, at least 4, at least 5, at least 6, at least 7, at least 8, at least 9, at least 10, etc.) such as GGGGS (SEQ ID NO: 108), GGGGT (SEQ ID NO: 78), SGGS (SEQ ID NO: 79), GGSG (SEQ ID NO: 80), GGSGG (SEQ ID NO: 81), GSGSG (SEQ ID NO: 82), GSGGG (SEQ ID NO: 83), GGGSG (SEQ ID NO: 84), and/or GSSSG (SEQ ID NO: 85); glycine-alanine polymers; alanine-serine polymers; and the like. Linker sequences may be of any length, such as from about 1 amino acid (e.g., glycine or serine) to about 20 amino acids (e.g., 20 amino acid glycine polymers or glycine-serine polymers), about 1 amino acid to about 15 amino acids, about 3 amino acids to about 12 amino acids, about 4 amino acids to about 10 amino acids, about 5 amino acids to about 9 amino acids, about 6 amino acids to about 8 amino acids, etc. In some embodiments, the linker is any of about 1, 2, 3, 4, 5, 6, 7, 8, 9, 10, 11, 12, 13, 14, 15, 16, 17, 18, 19, or 20 amino acids in length.

In some embodiments, the LU comprises an amino acid sequence selected from the group consisting of SEQ ID NOs: 8 and 10-16. SEQ ID NOs: 8 and 10-16 are provided in Table 3 below.

TABLE 3
Exemplary Linkage Unit Sequences (SEQ ID NOs: 8-36)

| SEQ ID NO: | LINKAGE UNIT (LU) SEQUENCES |
|---|---|
| 8 | SGRSAGGGGTPLGLAGSGGS |
| 10 | PLGLAGSGGS |
| 11 | GGGPLGLAGGGS |
| 12 | GGGPLGLAGGS |
| 13 | SGGGPLGLAGGS |
| 14 | SGRGPLGLAGGS |
| 15 | SGRGGPLGLAGGS |
| 16 | SGRGGGPLGLAGGS |

In some embodiments, the masking peptide (MP) comprises the structure, from N-terminus to C-terminus, of: (MU)-(LU), wherein LU comprises the structure (CS₁)-L₁ or (CS₁)-L₁-(CS₂)-L₂. In some embodiments, the masking peptide of the present disclosure comprises an amino acid sequence selected from the group consisting of SEQ ID NOS:43-65.

In some embodiments, the masking peptide (MP) comprises an MU set forth in any one of SEQ ID NOs: 1-7 and an LU set forth in any one of SEQ ID NOs: 8-16. In some embodiments, the MP comprises a sequence set forth in any one of SEQ ID NOs: 17-35. SEQ ID NOs: 17-35 are provided in Table 4 below.

TABLE 4
Exemplary Masking Peptide Sequences (SEQ ID NOs: 17-35)

| SEQ ID NO | MASKING PEPTIDE SEQUENCES |
|---|---|
| → | (N- to C-terminus) |
| 17 | EVGSYPTDLDACADAPNHCHFSGRSAGGGGTPLGLAGSGGS |
| 18 | EVGSYSSTHAHCHHSPANCISSGRSAGGGGTPLGLAGSGGS |
| 19 | EVGSYDTDYDFCPILRHRCDSSGRSAGGGGTPLGLAGSGGS |
| 20 | EVGSYNDYNYHCKWRPSRCHNSGRSAGGGGTPLGLAGSGGS |
| 21 | EVGSYYHDYDDCRVLPRRCFNSGRSAGGGGTPLGLAGSGGS |
| 22 | EVGSYSNNFASCLWRHRSCADSGRSAGGGGTPLGLAGSGGS |
| 23 | EVGSYTDNYDYCPRLRRKCYHSGRSAGGGGTPLGLAGSGGS |
| 24 | EVGSYDTDYDFCPILRHRCDSPLGLAGSGGS |
| 25 | EVGSYDTDYDFCPILRHRCDSSGRSAGGGGTPLGLAGSGGS |
| 26 | EVGSYNDYNYHCKWRPSRCHNPLGLAGSGGS |
| 27 | EVGSYNDYNYHCKWRPSRCHNSGRSAGGGGTPLGLAGSGGS |
| 28 | EVGSYTDNYDYCPRLRRKCYHPLGLAGSGGS |
| 29 | EVGSYTDNYDYCPRLRRKCYHSGRSAGGGGTPLGLAGSGGS |
| 30 | EVGSYTDNYDYCPRLRRKCYHGGGPLGLAGGGS |
| 31 | EVGSYPTDLDACADAPNHCHFGGGPLGLAGGS |
| 32 | EVGSYPTDLDACADAPNHCHFSGGGPLGLAGGS |
| 33 | EVGSYPTDLDACADAPNHCHFSGRGPLGLAGGS |
| 34 | EVGSYPTDLDACADAPNHCHFSGRGGPLGLAGGS |
| 35 | EVGSYPTDLDACADAPNHCHFSGRGGGPLGLAGGS |

*N-terminal unit sequences are in plain text; masking sequences are in bold underlined type; linkage units are in bold type

In some embodiments, the masked antibody (which is also referred to herein as a “masked anti-CD137 antibody”) comprises a VH set forth in SEQ ID NO: 52 and a VL set forth in SEQ ID NO: 53. In some embodiments, the masked antibody (which is also referred to herein as a “masked anti-CD137 antibody”) comprises a VH set forth in SEQ ID NO: 54 and a VL set forth in SEQ ID NO: 55. In some embodiments, the masked antibody (which is also referred to herein as a “masked anti-CD137 antibody”) comprises a VH set forth in SEQ ID NO: 56 and a VL set forth in SEQ ID NO: 57. In some embodiments, the masked anti-CD137 antibody comprises a VH set forth in SEQ ID NO: 52 and a VL set forth in any one of SEQ ID NOs: 58-76. SEQ ID NOs: 52-76 are provided in Tables 5A and 5B below.

TABLE 5A
VH and VL Sequences of Exemplary anti-CD137 antibodies

| | | |
|---|---|---|
| TY24122/ | VH | EVQLVESGGGLVQPGGSLRLSCAASGFSLSTGGVGVGWIRQAPGKGLEW |
| TY21242/ | | LALIDWADDKYYSPSLKSRLTISRDNSKNTLYLQLNSLRAEDTAVYYCA |
| | | RGGSDTVIGDWFAYWGQGTLVTVSS |
| | | (SEQ ID NO: 52) |
| TY23310/ | VL | DIQLTQSPSSLSASVGDRVTITCRASQSIGSYLAWYQQKPGKAPKLLIY |
| TY24118 | | DASNLETGVPSRFSGSGSGTDFTLTISSLQPEDFATYYCQQGYYLWTFG |
| | | QGTKVEIKR (SEQ ID NO: 53) |
| AG10058 | VH | EVQLVESGGGLVQPGGSLRLSCAASGFSLSTSGVGVGWIRQAPGKGLEW |
| | | LALIDWDDDKYYSPSLKSRLTISRDNSKNTLYLQLNSLRAEDTAVYYCA |
| | | RGGSDTVLGDWFAYWGQGTLVTVSS (SEQ ID NO: 54) |
| | VL | DIQLTQSPSSLSASVGDRVTITCRASQSVSPYLAWYQQKPGKAPKLLIY |
| | | DASSLESGVPSRFSGSGSGTDFTLTISSLQPEDFATYYCQQGYSLWTFG |
| | | QGTKVEIKR (SEQ ID NO: 55) |
| AG10059 | VH | EVQLVESGGGLVQPGGSLRLSCAASGYSITSGHYWAWIRQAPGKGLEWV |
| | | SSISGYGSTTYYADSVKGRFTISRDNSKNTLYLQLNSLRAEDTAVYYCA |
| | | RGGSDAVLGDWFAYWGQGTLVTVSS (SEQ ID NO: 56) |
| | VL | DIQLTQSPSSLSASVGDRVTITCRASQGIGSFLAWYQQKPGKAPKLLIY |
| | | DASNLETGVPSRFSGSGSGTDFTLTISSLQPEDFATYYCQQGYYLWTFG |
| | | QGTKVEIKR (SEQ ID NO: 57) |

TABLE 5B
VH and VL Sequences of Exemplary Masked anti-CD137 antibodies*
(N- to C-terminus)

| | | |
|---|---|---|
| VH (same for | VH | EVQLVESGGGLVQPGGSLRLSCAASGFSLSTGGVGVGWIRQA |
| all masked | | PGKGLEWLALIDWADDKYYSPSLKSRLTISRDNSKNTLYLQL |
| anti-CD137 | | NSLRAEDTAVYYCARGGSDTVIGDWFAYWGQGTLVTVSS |
| antibodies in | | (SEQ ID NO: 52) |
| Table 5B) | | |
| TY25368 | VL | EVGSYPTDLDACADAPNHCHFSGRGGPLGLAGGSDIQLTQSPS |
| and | | SLSASVGDRVTITCRASQSIGSYLAWYQQKPGKAPKLLIYDASNL |
| TY25366 | | ETGVPSRFSGSGSGTDFTLTISSLQPEDFATYYCQQGYYLWTFGQG |
| | | TKVEIKR (SEQ ID NO: 58) |
| TY22586 | VL | EVGSYPTDLDACADAPNHCHFSGRSAGGGGTPLGLAGSGGS |
| | | DIQLTQSPSSLSASVGDRVTITCRASQSIGSYLAWYQQKPGKAPKL |
| | | LIYDASNLETGVPSRFSGSGSGTDFTLTISSLQPEDFATYYCQQGYY |
| | | LWTFGQGTKVEIKR (SEQ ID NO: 59) |
| TY22591 | VL | EVGSYSSTHAHCHHSPANCISSGRSAGGGGTPLGLAGSGGS |
| | | DIQLTQSPSSLSASVGDRVTITCRASQSIGSYLAWYQQKPGKAPKLL |
| | | IYDASNLETGVPSRFSGSGSGTDFTLTISSLQPEDFATYYCQQGYYL |
| | | WTFGQGTKVEIKR (SEQ ID NO: 60) |
| TY22594 | VL | EVGSYDTDYDFCPILRHRCDSSGRSAGGGGTPLGLAGSGGS |
| | | DIQLTQSPSSLSASVGDRVTITCRASQSIGSYLAWYQQKPGKAPKLL |
| | | IYDASNLETGVPSRFSGSGSGTDFTLTISSLQPEDFATYYCQQGYYL |
| | | WTFGQGTKVEIKR (SEQ ID NO: 61) |
| TY22595 | VL | EVGSYNDYNYHCKWRPSRCHNSGRSAGGGGTPLGLAGSG |
| | | GSDIQLTQSPSSLSASVGDRVTITCRASQSIGSYLAWYQQKPGKAP |
| | | KLLIYDASNLETGVPSRFSGSGSGTDFTLTISSLQPEDFATYYCQQG |
| | | YYLWTFGQGTKVEIKR (SEQ ID NO: 62) |
| TY22596 | VL | EVGSYYHDYDDCRVLPRRCFNSGRSAGGGGTPLGLAGSGG |
| | | SDIQLTQSPSSLSASVGDRVTITCRASQSIGSYLAWYQQKPGKAPKL |
| | | LIYDASNLETGVPSRFSGSGSGTDFTLTISSLQPEDFATYYCQQGYY |
| | | LWTFGQGTKVEIKR (SEQ ID NO: 63) |
| TY22598 | VL | EVGSYSNNFASCLWRHRSCADSGRSAGGGGTPLGLAGSGG |
| | | SDIQLTQSPSSLSASVGDRVTITCRASQSIGSYLAWYQQKPGKAPKL |
| | | LIYDASNLETGVPSRFSGSGSGTDFTLTISSLQPEDFATYYCQQGYY |
| | | LWTFGQGTKVEIKR (SEQ ID NO: 64) |
| TY22599 | VL | EVGSYTDNYDYCPRLRRKCYHSGRSAGGGGTPLGLAGSG |
| | | GSDIQLTQSPSSLSASVGDRVTITCRASQSIGSYLAWYQQKPGKAP |
| | | KLLIYDASNLETGVPSRFSGSGSGTDFTLTISSLQPEDFATYYCQQG |
| | | YYLWTFGQGTKVEIKR (SEQ ID NO: 65) |
| TY23565 | VL | EVGSYDTDYDFCPILRHRCDSPLGLAGSGGSDIQLTQSPSSLS |
| and | | ASVGDRVTITCRASQSIGSYLAWYQQKPGKAPKLLIYDASNLETGV |
| TY23571 | | PSRFSGSGSGTDFTLTISSLQPEDFATYYCQQGYYLWTFGQGTKVE |
| | | IKR (SEQ ID NO: 66) |
| TY23568 | VL | EVGSYDTDYDFCPILRHRCDSSGRSAGGGGTPLGLAGSGGS |
| | | DIQLTQSPSSLSASVGDRVTITCRASQSIGSYLAWYQQKPGKAPKLL |
| | | IYDASNLETGVPSRFSGSGSGTDFTLTISSLQPEDFATYYCQQGYYL |
| | | WTFGQGTKVEIKR (SEQ ID NO: 67) |
| TY23566, | VL | EVGSYNDYNYHCKWRPSRCHNPLGLAGSGGSDIQLTQSPSS |
| TY23572, | | LSASVGDRVTITCRASQSIGSYLAWYQQKPGKAPKLLIYDASNLETG |
| And | | VPSRFSGSGSGTDFTLTISSLQPEDFATYYCQQGYYLWTFGQGTKV |
| TY25370 | | EIKR (SEQ ID NO: 68) |
| TY23569 | VL | EVGSYNDYNYHCKWRPSRCHNSGRSAGGGGTPLGLAGSG |
| | | GSDIQLTQSPSSLSASVGDRVTITCRASQSIGSYLAWYQQKPGKAP |
| | | KLLIYDASNLETGVPSRFSGSGSGTDFTLTISSLQPEDFATYYCQQG |
| | | YYLWTFGQGTKVEIKR (SEQ ID NO: 69) |
| TY23567, | VL | EVGSYTDNYDYCPRLRRKCYHPLGLAGSGGSDIQLTQSPSSL |
| TY25371, | | SASVGDRVTITCRASQSIGSYLAWYQQKPGKAPKLLIYDASNLETGV |
| and | | PSRFSGSGSGTDFTLTISSLQPEDFATYYCQQGYYLWTFGQGTKVE |
| TY23573 | | IKR (SEQ ID NO: 70) |
| TY23570 | VL | EVGSYTDNYDYCPRLRRKCYHSGRSAGGGGTPLGLAGSGGS |
| | | DIQLTQSPSSLSASVGDRVTITCRASQSIGSYLAWYQQKPGKAP |
| | | KLLIYDASNLETGVPSRFSGSGSGTDFTLTISSLQPEDFATYYCQQG |
| | | YYLWTFGQGTKVEIKR (SEQ ID NO: 71) |
| TY25372 | VL | EVGSYTDNYDYCPRLRRKCYHGGGPLGLAGGGSDIQLTQSPSS |
| | | LSASVGDRVTITCRASQSIGSYLAWYQQKPGKAPKLLIYDASNLE |
| | | TGVPSRFSGSGSGTDFTLTISSLQPEDFATYYCQQGYYLWTFGQGT |
| | | KVEIKR (SEQ ID NO: 72) |
| TY25153 | VL | EVGSYPTDLDACADAPNHCHFGGGPLGLAGGSDIQLTQSPSSLS |
| | | ASVGDRVTITCRASQSIGSYLAWYQQKPGKAPKLLIYDASNLETG |
| | | VPSRFSGSGSGTDFTLTISSLQPEDFATYYCQQGYYLWTFGQGTKV |
| | | EIKR (SEQ ID NO: 73) |
| TY25154 | VL | EVGSYPTDLDACADAPNHCHFSGGGPLGLAGGSDIQLTQSPSS |
| | | LSASVGDRVTITCRASQSIGSYLAWYQQKPGKAPKLLIYDASNLE |
| | | TGVPSRFSGSGSGTDFTLTISSLQPEDFATYYCQQGYYLWTFGQGT |
| | | KVEIKR (SEQ ID NO: 74) |
| TY25155 | VL | EVGSYPTDLDACADAPNHCHFSGRGPLGLAGGSDIQLTQSPSSL |
| | | SASVGDRVTITCRASQSIGSYLAWYQQKPGKAPKLLIYDASNLET |
| | | GVPSRFSGSGSGTDFTLTISSLQPEDFATYYCQQGYYLWTFGQGT |
| | | KVEIKR (SEQ ID NO: 75) |
| TY25369 | VL | EVGSYPTDLDACADAPNHCHFSGRGGGPLGLAGGSDIQLTQSP |
| | | SSLSASVGDRVTITCRASQSIGSYLAWYQQKPGKAPKLLIYDASN |
| | | LETGVPSRFSGSGSGTDFTLTISSLQPEDFATYYCQQGYYLWTFGQ |
| | | GTKVEIKR (SEQ ID NO: 76) |

* N-terminal unit is in plain text, masking unit is in underlined bold text, linkage unit is in bold text, VL sequence is in italicized text.

In some embodiments, the masked anti-CD137 antibody comprises an MP that comprises SEQ ID NO: 34; a VH that comprises a CDR-H1 set forth in TGGVGVG (SEQ ID NO: 36), a CDR-H2 set forth in LIDWADDKYYSPSLKS (SEQ ID NO: 37), and CDR-H3 set forth in GGSDTVIGDWFAY (SEQ ID NO: 38); and a VL that comprises a CDR-L1 set forth in RASQSIGSYLA (SEQ ID NO: 39), a CDR-L2 set forth in DASNLET (SEQ ID NO: 40), and a CDR-L3 set forth in QQGYYLWT (SEQ ID NO: 41). In some embodiments, the masked antibody comprises an MP that comprises SEQ ID NO: 34, a VH that comprises SEQ ID NO: 52, and a VL that comprises SEQ ID NO: 53. IN some embodiments, the masked antibody comprises a VH that comprises SEQ ID NO: 52 and a VL that comprises SEQ ID NO: 58.

In some embodiments, the masked anti-CD137 antibody comprises a full length antibody light chain, e.g., a kappa or lambda light chain. Additionally or alternatively, in some embodiments, the anti-CD137 antibody comprises a full-length antibody heavy chain. The antibody heavy chain may be in any class, such as IgG, IgM, IgE, IgA, or IgD. In some embodiments, the antibody heavy chain is in the IgG class, such as IgG1, IgG2, IgG3, or IgG4 subclass. An antibody heavy chain described herein may be converted from one class or subclass to another class or subclass using RECTIFIED SHEET (RULE 91) ISA/CN methods known in the art. In some embodiments, the masked anti-CD137 antibody is or comprises a full length antibody that comprises an Fc region, e.g., a human Fc region or a variant thereof. In some embodiments, the human Fc region is a human IgG1 Fc region, a human IgG2 Fc region, a human IgG4 Fc region, or a variant of any one of the preceding. In some embodiments the variant Fc region comprises one or more amino acid substitutions, insertions, or deletions relative to the wild type human Fc region from which the variant is derived. In some embodiments, the masked anti-CD137 antibody comprises a variant of a human IgG1 Fc region. In some embodiments, the IgG1 Fc variant comprises one or more amino acid substitutions that increases the affinity of the Fc variant for FcγRIIb. In some embodiments, the variant of the human IgG1 Fc region comprises substitution(s) selected from the group consisting of: G236D; L328F; S239D; S267E; G236D and S267E; S239D and S267E; S267E and L328S; and S267E and L328F, wherein amino acid numbering is according to the EU index (see, e.g., Edelman et al., 1969, Proc Natl Acad Sci USA 63: 78-85). The preceding substitutions are described in Chu et al. (2008) Mol Immunol. 45(15):3926-33. Additionally or alternatively, in some embodiments, the variant of the human IgG1 Fc region comprises substitution(s) selected from the group consisting of: E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R, wherein amino acid numbering is according to the EU index. The preceding substitutions are described in Mimoto et al. (2013) Protein Eng Des Sel. 26(10):589-98. Additionally or alternatively, in some embodiments, the variant of the human IgG1 Fc region comprises an S2657A substitution (see Buschor et al. (2014) Int Arch Allergy Immunol. 163(3):206-14), wherein amino acid numbering is according to the EU index. Additionally or alternatively, in some embodiments, the variant of the human IgG1 Fc region comprises a T437R and/or a K248E substitution (see Zhang et al. (2017) MAbs. 9(7):1129-1142), wherein amino acid numbering is according to the EU index. In some embodiments, the masked anti-CD137 antibody comprises a variant of a human IgG4 Fc region. In some embodiments, the IgG4 Fc variant comprises one or more amino acid substitutions that increases the affinity of the Fc variant for FcγRIIb. In some embodiments, the variant of the human IgG4 Fc region comprises substitution(s) selected from the group consisting of: G236D; L328F; S239D; S267E; G236D and S267E; S239D and S267E; S267E and L328S; and S267E and L328F, wherein amino acid numbering is according to the EU index. Additionally or alternatively, in some embodiments, the variant of the human IgG4 Fc region comprises substitution(s) selected from the group consisting of: E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R, wherein amino acid numbering is according to the EU index. Additionally or alternatively, in some embodiments, the variant of the human IgG4 Fc region comprises an S2657A substitution, wherein amino acid numbering is according to the EU index. Additionally or alternatively, in some embodiments, the variant of the human IgG1 Fc region comprises a T437R and/or a K248E substitution wherein amino acid numbering is according to the EU index.

In some embodiments, the masked anti-CD137 antibody comprises the masking peptide of SEQ ID NO: 34, a VH domain set forth in SEQ ID NO: 52, and a VL domain set forth in SEQ ID NO: 53. In some embodiments, the masked anti-CD137 antibody further comprise a human IgG1 domain or a variant thereof that comprises one or more substitution mutation(s). In some embodiments the IgG1 variant comprises substitution(s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index. In some embodiments, the masked anti-CD137 antibody further comprise a human IgG4 domain or a variant thereof that comprises one or more substitution mutation(s). In some embodiments the IgG4 variant comprises substitution(s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index.

In some embodiments, the masked anti-CD137 antibody comprises a heavy chain constant region that comprises an amino acid sequence set forth in any one of SEQ ID NOs: 11I1-118. See Table 6 below.

TABLE 6
Exemplary Heavy Chain Constant Region Sequences

| SEQ ID NO: | Sequence (N-to-C) | Description |
|---|---|---|
| 111 | ASTKGPSVFPLAPSSKSTSGGTAALGCLVKDYFPEPVT | WT Human IgG1 constant |
| | VSWNSGALTSGVHTFPAVLQSSGLYSLSSVVTVPSSSL | region |
| | GTQTYICNVNHKPSNTKVDKKVEPKSCDKTHTCPPCP | |
| | APELLGGPSVFLFPPKPKDTLMISRTPEVTCVVVDVS | |
| | HEDPEVKFNWYVDGVEVHNAKTKPREEQYNSTYRVV | |
| | SVLTVLHQDWLNGKEYKCKVSNKALPAPIEKTISKAK | |
| | GQPREPQVYTLPPSRDELTKNQVSLTCLVKGFYPSDI | |
| | AVEWESNGQPENNYKTTPPVLDSDGSFFLYSKLTVDK | |
| | SRWQQGNVFSCSVMHEALHNHYTQKSLSLSPGK | |
| 112 | ASTKGPSVFPLAPSSKSTSGGTAALGCLVKDYFPEPVT | WT Human IgG1 constant |
| | VSWNSGALTSGVHTFPAVLQSSGLYSLSSVVTVPSSSL | region |
| | GTQTYICNVNHKPSNTKVDKKVEPKSCDKTHTCPPCP | (No C-terminal K) |
| | APELLGGPSVFLFPPKPKDTLMISRTPEVTCVVVDVS | |
| | HEDPEVKFNWYVDGVEVHNAKTKPREEQYNSTYRVV | |
| | SVLTVLHQDWLNGKEYKCKVSNKALPAPIEKTISKAK | |
| | GQPREPQVYTLPPSRDELTKNQVSLTCLVKGFYPSDI | |
| | AVEWESNGQPENNYKTTPPVLDSDGSFFLYSKLTVDK | |
| | SRWQQGNVFSCSVMHEALHNHYTQKSLSLSPG | |
| 113 | ASTKGPSVFPLAPSSKSTSGGTAALGCLVKDYFPEPVT | Human IgG1 variant |
| | VSWNSGALTSGVHTFPAVLQSSGLYSLSSVVTVPSSSL | comprising S267E and |
| | GTQTYICNVNHKPSNTKVDKKVEPKSCDKTHTCPPCPA | L328F substitutions (EU |
| | PELLGGPSVFLFPPKPKDTLMISRTPEVTCVVVDVEH | numbering) |
| | EDPEVKFNWYVDGVEVHNAKTKPREEQYNSTYRVVSV | |
| | LTVLHQDWLNGKEYKCKVSNKAFPAPIEKTISKAKGQ | |
| | PREPQVYTLPPSRDELTKNQVSLTCLVKGFYPSDIAV | |
| | EWESNGQPENNYKTTPPVLDSDGSFFLYSKLTVDKS | |
| | RWQQGNVFSCSVMHEALHNHYTQKSLSLSPGK | |
| 114 | ASTKGPSVFPLAPSSKSTSGGTAALGCLVKDYFPEPVT | Human IgG1 variant |
| | VSWNSGALTSGVHTFPAVLQSSGLYSLSSVVTVPSSSL | comprising S267E and |
| | GTQTYICNVNHKPSNTKVDKKVEPKSCDKTHTCPPCP | L328F substitutions (EU |
| | APELLGGPSVFLFPPKPKDTLMISRTPEVTCVVVDVE | numbering) |
| | HEDPEVKFNWYVDGVEVHNAKTKPREEQYNSTYRVV | (No C-terminal K) |
| | SVLTVLHQDWLNGKEYKCKVSNKAFPAPIEKTISKAK | |
| | GQPREPQVYTLPPSRDELTKNQVSLTCLVKGFYPSDI | |
| | AVEWESNGQPENNYKTTPPVLDSDGSFFLYSKLTVDK | |
| | SRWQQGNVFSCSVMHEALHNHYTQKSLSLSPG | |
| 115 | ASTKGPSVFPLAPCSRSTSESTAALGCLVKDYFPEPVT | WT Human IgG4 constant |
| | VSWNSGALTSGVHTFPAVLQSSGLYSLSSVVTVPSSSL | region |
| | GTKTYTCNVDHKPSNTKVDKRVESKYGPPCPPCPAPE | |
| | FLGGPSVFLFPPKPKDTLMISRTPEVTCVVVDVSQEDP | |
| | EVQFNWYVDGVEVHNAKTKPREEQFNSTYRVVSVLT | |
| | VLHQDWLNGKEYKCKVSNKGLPSSIEKTISKAKGQPR | |
| | EPQVYTLPPSQEEMTKNQVSLTCLVKGFYPSDIAVEW | |
| | ESNGQPENNYKTTPPVLDSDGSFFLYSRLTVDKSRWQ | |
| | EGNVFSCSVMHEALHNHYTQKSLSLSLGK | |
| 116 | ASTKGPSVFPLAPCSRSTSESTAALGCLVKDYFPEPVT | WT Human IgG4 constant |
| | VSWNSGALTSGVHTFPAVLQSSGLYSLSSVVTVPSSSL | region |
| | GTKTYTCNVDHKPSNTKVDKRVESKYGPPCPPCPAPE | (No C-terminal K) |
| | FLGGPSVFLFPPKPKDTLMISRTPEVTCVVVDVSQEDP | |
| | EVQFNWYVDGVEVHNAKTKPREEQFNSTYRVVSVLT | |
| | VLHQDWLNGKEYKCKVSNKGLPSSIEKTISKAKGQPR | |
| | EPQVYTLPPSQEEMTKNQVSLTCLVKGFYPSDIAVEW | |
| | ESNGQPENNYKTTPPVLDSDGSFFLYSRLTVDKSRWQ | |
| | EGNVFSCSVMHEALHNHYTQKSLSLSLG | |
| 117 | ASTKGPSVFPLAPCSRSTSESTAALGCLVKDYFPEPVT | Human IgG4 variant |
| | VSWNSGALTSGVHTFPAVLQSSGLYSLSSVVTVPSSSL | comprising S267E and |
| | GTKTYTCNVDHKPSNTKVDKRVESKYGPPCPPCPAPE | L328F substitutions (EU |
| | FLGGPSVFLFPPKPKDTLMISRTPEVTCVVVDVEQEDP | numbering) |
| | EVQFNWYVDGVEVHNAKTKPREEQFNSTYRVVSVLT | |
| | VLHQDWLNGKEYKCKVSNKGFPSSIEKTISKAKGQPR | |
| | EPQVYTLPPSQEEMTKNQVSLTCLVKGFYPSDIAVEW | |
| | ESNGQPENNYKTTPPVLDSDGSFFLYSRLTVDKSRWQ | |
| | EGNVFSCSVMHEALHNHYTQKSLSLSLGK | |
| 118 | ASTKGPSVFPLAPCSRSTSESTAALGCLVKDYFPEPVT | Human IgG4 variant |
| | VSWNSGALTSGVHTFPAVLQSSGLYSLSSVVTVPSSSL | comprising S267E and |
| | GTKTYTCNVDHKPSNTKVDKRVESKYGPPCPPCPAPE | L328F substitutions (EU |
| | FLGGPSVFLFPPKPKDTLMISRTPEVTCVVVDVEQEDP | numbering) |
| | EVQFNWYVDGVEVHNAKTKPREEQFNSTYRVVSVLT | (No C-terminal K) |
| | VLHQDWLNGKEYKCKVSNKGFPSSIEKTISKAKGQPR | |
| | EPQVYTLPPSQEEMTKNQVSLTCLVKGFYPSDIAVEW | |
| | ESNGQPENNYKTTPPVLDSDGSFFLYSRLTVDKSRWQ | |
| | EGNVFSCSVMHEALHNHYTQKSLSLSLG | |

In some embodiments, the masked anti-CD137 antibody comprises a heavy chain comprising any one of SEQ ID NOs: 88-95. Additionally or alternatively, in some embodiments, the masked anti-CD137 antibody comprises a light chain comprising any one of SEQ ID NOs: 96-109 and 119-122.

In some embodiments, the masked anti-CD137 antibody comprises a heavy chain comprising SEQ ID NO: 88 or 89 and a light chain comprising SEQ ID NO: 109. In some embodiments, the masked anti-CD137 antibody comprises a heavy chain comprising SEQ ID NO: 92 or 93 and a light chain comprising SEQ ID NO: 109. In some embodiments, the masked anti-CD137 antibody comprises a heavy chain comprising SEQ ID NO: 90 or 91 and a light chain comprising SEQ ID NO: 109. In some embodiments, the masked anti-CD137 antibody comprises a heavy chain comprising SEQ ID NO: 94 or 95 and a light chain comprising SEQ ID NO: 109. In some embodiments, the masked anti-CD137 antibody comprises a heavy chain comprising SEQ ID NO: 94 or 95 and a light chain comprising SEQ ID NO: 96. In some embodiments, the masked anti-CD137 antibody comprises a heavy chain comprising SEQ ID NO: 92 or 93 and a light chain comprising SEQ ID NO: 96. In some embodiments, the masked anti-CD137 antibody comprises a heavy chain comprising SEQ ID NO: 88 or 89 and a light chain comprising SEQ ID NO: 97. In some embodiments, the masked anti-CD137 antibody comprises a heavy chain comprising SEQ ID NO: 88 or 89 and a light chain comprising SEQ ID NO: 98. In some embodiments, the masked anti-CD137 antibody comprises a heavy chain comprising SEQ ID NO: 88 or 89 and a light chain comprising SEQ ID NO: 119. In some embodiments, the masked anti-CD137 antibody comprises a heavy chain comprising SEQ ID NO: 88 or 89 and a light chain comprising SEQ ID NO: 100. In some embodiments, the masked anti-CD137 antibody comprises a heavy chain comprising SEQ ID NO: 88 or 89 and a light chain comprising SEQ ID NO: 120. In some embodiments, the masked anti-CD137 antibody comprises a heavy chain comprising SEQ ID NO: 88 or 89 and a light chain comprising SEQ ID NO: 121. In some embodiments, the masked anti-CD137 antibody comprises a heavy chain comprising SEQ ID NO: 88 or 89 and a light chain comprising SEQ ID NO: 102. In some embodiments, the masked anti-CD137 antibody comprises a heavy chain comprising SEQ ID NO: 88 or 89 and a light chain comprising SEQ ID NO: 103. In some embodiments, the masked anti-CD137 antibody comprises a heavy chain comprising SEQ ID NO: 90 or 91 and a light chain comprising SEQ ID NO: 98. In some embodiments, the masked anti-CD137 antibody comprises a heavy chain comprising SEQ ID NO: 90 or 91 and a light chain comprising SEQ ID NO: 1. In some embodiments, the masked anti-CD137 antibody comprises a heavy chain comprising SEQ ID NO: 88 or 89 and a light chain comprising SEQ ID NO: 99. In some embodiments, the masked anti-CD137 antibody comprises a heavy chain comprising SEQ ID NO: 90 or 91 and a light chain comprising SEQ ID NO: 100. In some embodiments, the masked anti-CD137 antibody comprises a heavy chain comprising SEQ ID NO: 90 or 91 and a light chain comprising SEQ ID NO: 99. In some embodiments, the masked anti-CD137 antibody comprises a heavy chain comprising SEQ ID NO: 94 or 95 and a light chain comprising SEQ ID NO: 99. In some embodiments, the masked anti-CD137 antibody comprises a heavy chain comprising SEQ ID NO: 88 or 89 and a light chain comprising SEQ ID NO: 101. In some embodiments, the masked anti-CD137 antibody comprises a heavy chain comprising SEQ ID NO: 94 or 95 and a light chain comprising SEQ ID NO: 101. In some embodiments, the masked anti-CD137 antibody comprises a heavy chain comprising SEQ ID NO: 90 or 91 and a light chain comprising SEQ ID NO: 102. In some embodiments, the masked anti-CD137 antibody comprises a heavy chain comprising SEQ ID NO: 90 or 91 and a light chain comprising SEQ ID NO: 101. In some embodiments, the masked anti-CD137 antibody comprises a heavy chain comprising SEQ ID NO: 94 or 95 and a light chain comprising SEQ ID NO: 104. In some embodiments, the masked anti-CD137 antibody comprises a heavy chain comprising SEQ ID NO: 88 or 89 and a light chain comprising SEQ ID NO: 105. In some embodiments, the masked anti-CD137 antibody comprises a heavy chain comprising SEQ ID NO: 88 or 89 and a light chain comprising SEQ ID NO: 106. In some embodiments, the masked anti-CD137 antibody comprises a heavy chain comprising SEQ ID NO: 88 or 89 and a light chain comprising SEQ ID NO: 107. In some embodiments, the masked anti-CD137 antibody comprises a heavy chain comprising SEQ ID NO: 94 or 94 and a light chain comprising SEQ ID NO: 122. The amino acid sequences of SEQ ID NOs: 88-95-109 and 119-122 are provided in Tables 7A and 7B below.

TABLE 7A
Heavy Chain and Light Chain Sequences of Exemplary Non-Masked anti-CD137
Antibodies (N- to C-terminus)

| | | |
|---|---|---|
| TY21242 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | DIQLTQSPSSLSASVGDRVTITCRASQSIGSYLAWYQQKPGKAPKLLIYDASNLE |
| | | TGVPSRFSGSGSGTDFTLTISSLQPEDFATYYCQQGYYLWTFGQGTKVEIKRT |
| | | VAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKS |
| | | FNRGEC (SEQ ID NO: 109) |
| TY24122 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVEQEDPEVQFNWYVDGVEVHNAKTKPREEQFNSTYRVV |
| | | SVLTVLHQDWLNGKEYKCKVSNKGFPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 92) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVEQEDPEVQFNWYVDGVEVHNAKTKPREEQFNSTYRVV |
| | | SVLTVLHQDWLNGKEYKCKVSNKGFPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 93) |
| | LC | DIQLTQSPSSLSASVGDRVTITCRASQSIGSYLAWYQQKPGKAPKLLIYDASNLE |
| | | TGVPSRFSGSGSGTDFTLTISSLQPEDFATYYCQQGYYLWTFGQGTKVEIKRT |
| | | VAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKS |
| | | FNRGEC (SEQ ID NO: 109) |
| TY23310 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 90) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 91) |
| | LC | DIQLTQSPSSLSASVGDRVTITCRASQSIGSYLAWYQQKPGKAPKLLIYDASNLE |
| | | TGVPSRFSGSGSGTDFTLTISSLQPEDFATYYCQQGYYLWTFGQGTKVEIKRT |
| | | VAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKS |
| | | FNRGEC (SEQ ID NO: 109) |
| TY24118 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKENWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 94) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKFNWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 95) |
| | LC | DIQLTQSPSSLSASVGDRVTITCRASQSIGSYLAWYQQKPGKAPKLLIYDASNLE |
| | | TGVPSRFSGSGSGTDFTLTISSLQPEDFATYYCQQGYYLWTFGQGTKVEIKRT |
| | | VAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKS |
| | | FNRGEC (SEQ ID NO: 109) |

For LC, VL sequence is in italicized text, CL is in double underlined text
For HC, VH sequence is in italicized text, CH is in double underlined text

TABLE 7B
Heavy Chain and Light Chain Sequences of Exemplary Masked anti-CD137 Antibodies
(N- to C-terminus)

| | | |
|---|---|---|
| TY25368 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKFNWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 94) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKFNWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 95) |
| | LC | EVGSYPTDLDACADAPNHCHFSGRGGPLGLAGGSDIQLTQSPSSLSASV |
| | | GDRVTITCRASQSIGSYLAWYQQKPGKAPKLLIYDASNLETGVPSRFSGSGSGT |
| | | DFTLTISSLQPEDFATYYCQQGYYLWTFGQGTKVEIKRTVAAPSVFIFPPSDE |
| | | QLKSGTASVVCLLNNFYPREAKVQWKVDNALQSGNSQESVTEQDSKDS |
| | | TYSLSSTLTLSKADYEKHKVYACEVTHQGLSSPVTKSFNRGEC (SEQ ID |
| | | NO: 96) |
| TY25366 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVEQEDPEVQFNWYVDGVEVHNAKTKPREEQFNSTYRVV |
| | | SVLTVLHQDWLNGKEYKCKVSNKGFPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 92) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVEQEDPEVQFNWYVDGVEVHNAKTKPREEQFNSTYRVV |
| | | SVLTVLHQDWLNGKEYKCKVSNKGFPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 93) |
| | LC | EVGSYPTDLDACADAPNHCHFSGRGGPLGLAGGSDIQLTQSPSSLSASV |
| | | GDRVTITCRASQSIGSYLAWYQQKPGKAPKLLIYDASNLETGVPSRFSGSGSGT |
| | | DFTLTISSLQPEDFATYYCQQGYYLWTFGQGTKVEIKRTVAAPSVFIFPPSDE |
| | | QLKSGTASVVCLLNNFYPREAKVQWKVDNALQSGNSQESVTEQDSKDS |
| | | TYSLSSTLTLSKADYEKHKVYACEVTHQGLSSPVTKSFNRGEC (SEQ ID |
| | | NO: 96) |
| TY22586 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYPTDLDACADAPNHCHFSGRSAGGGGTPLGLAGSGGSDIQLTQS |
| | | PSSLSASVGDRVTITCRASQSIGSYLAWYQQKPGKAPKLLIYDASNLETGVPSRF |
| | | SGSGSGTDFTLTISSLQPEDFATYYCQQGYYLWTFGQGTKVEIKRTVAAPSVF |
| | | IFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| | | (SEQ ID NO: 97) |
| TY22591 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYSSTHAHCHHSPANCISSGRSAGGGGTPLGLAGSGGSDIQLTQS |
| | | PSSLSASVGDRVTITCRASQSIGSYLAWYQQKPGKAPKLLIYDASNLETG |
| | | VPSRFSGSGSGTDFTLTISSLQPEDFATYYCQQGYYLWTFGQGTKVEIKR |
| | | TVAAPSVFIFPPSDEQLKSGTASVVCLLNNFYPREAKVQWKVDNALQSG |
| | | NSQESVTEQDSKDSTYSLSSTLTLSKADYEKHKVYACEVTHQGLSSPVTK |
| | | SFNRGEC (SEQ ID NO: 123) |
| TY22594 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYDTDYDFCPILRHRCDSSGRSAGGGGTPLGLAGSGGSDIQLTQS |
| | | PSSLSASVGDRVTITCRASQSIGSYLAWYQQKPGKAPKLLIYDASNLETGVPSRF |
| | | SGSGSGTDFTLTISSLQPEDFATYYCQQGYYLWTFGQGTKVEIKRTVAAPSVF |
| | | IFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| | | (SEQ ID NO: 119) |
| TY22595 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYNDYNYHCKWRPSRCHNSGRSAGGGGTPLGLAGSGGSDIQLTQ |
| | | SPSSLSASVGDRVTITCRASQSIGSYLAWYQQKPGKAPKLLIYDASNLETGVPSR |
| | | FSGSGSGTDFTLTISSLQPEDFATYYCQQGYYLWTFGQGTKVEIKRTVAAPSV |
| | | FIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| | | (SEQ ID NO: 100) |
| TY22596 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYYHDYDDCRVLPRRCFNSGRSAGGGGTPLGLAGSGGSDIQLTQ |
| | | SPSSLSASVGDRVTITCRASQSIGSYLAWYQQKPGKAPKLLIYDASNLETGVPSR |
| | | FSGSGSGTDFTLTISSLQPEDFATYYCQQGYYLWTFGQGTKVEIKRTVAAPSV |
| | | FIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| | | (SEQ ID NO: 120) |
| TY22598 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYSNNFASCLWRHRSCADSGRSAGGGGTPLGLAGSGGSDIQLTQS |
| | | PSSLSASVGDRVTITCRASQSIGSYLAWYQQKPGKAPKLLIYDASNLETGVPSRF |
| | | SGSGSGTDFTLTISSLQPEDFATYYCQQGYYLWTFGQGTKVEIKRTVAAPSVF |
| | | IFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| | | (SEQ ID NO: 121) |
| TY22599 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYTDNYDYCPRLRRKCYHSGRSAGGGGTPLGLAGSGGSDIQLTQ |
| | | SPSSLSASVGDRVTITCRASQSIGSYLAWYQQKPGKAPKLLIYDASNLETGVPSR |
| | | FSGSGSGTDFTLTISSLQPEDFATYYCQQGYYLWTFGQGTKVEIKRTVAAPSV |
| | | FIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| | | (SEQ ID NO: 102) |
| TY23565 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYDTDYDFCPILRHRCDSPLGLAGSGGSDIQLTQSPSSLSASVGDRV |
| | | TITCRASQSIGSYLAWYQQKPGKAPKLLIYDASNLETGVPSRFSGSGSGTDFTL |
| | | TISSLQPEDFATYYCQQGYYLWTFGQGTKVEIKRTVAAPSVFIFPPSDEQLKS |
| | | GTASVVCLLNNFYPREAKVQWKVDNALQSGNSQESVTEQDSKDSTYSL |
| | | SSTLTLSKADYEKHKVYACEVTHQGLSSPVTKSFNRGEC (SEQ ID NO: |
| | | 103) |
| TY23568 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 90) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 91) |
| | LC | EVGSYDTDYDFCPILRHRCDSSGRSAGGGGTPLGLAGSGGSDIQLTQS |
| | | PSSLSASVGDRVTITCRASQSIGSYLAWYQQKPGKAPKLLIYDASNLETGVPSRF |
| | | SGSGSGTDFTLTISSLQPEDFATYYCQQGYYLWTFGQGTKVEIKRTVAAPSVF |
| | | IFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| | | (SEQ ID NO: 98) |
| TY23571 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 90) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 91) |
| | LC | EVGSYDTDYDFCPILRHRCDSPLGLAGSGGSDIQLTQSPSSLSASVGDRV |
| | | TITCRASQSIGSYLAWYQQKPGKAPKLLIYDASNLETGVPSRFSGSGSGTDFTL |
| | | TISSLQPEDFATYYCQQGYYLWTFGQGTKVEIKRTVAAPSVFIFPPSDEQLKS |
| | | GTASVVCLLNNFYPREAKVQWKVDNALQSGNSQESVTEQDSKDSTYSL |
| | | SSTLTLSKADYEKHKVYACEVTHQGLSSPVTKSFNRGEC (SEQ ID NO: |
| | | 103) |
| TY23566 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYNDYNYHCKWRPSRCHNPLGLAGSGGSDIQLTQSPSSLSASVGDR |
| | | VTITCRASQSIGSYLAWYQQKPGKAPKLLIYDASNLETGVPSRFSGSGSGTDFT |
| | | LTISSLQPEDFATYYCQQGYYLWTFGQGTKVEIKRTVAAPSVFIFPPSDEQLK |
| | | SGTASVVCLLNNFYPREAKVQWKVDNALQSGNSQESVTEQDSKDSTYS |
| | | LSSTLTLSKADYEKHKVYACEVTHQGLSSPVTKSFNRGEC (SEQ ID NO: |
| | | 99) |
| TY23569 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKENWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 90) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKENWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 91) |
| | LC | EVGSYNDYNYHCKWRPSRCHNSGRSAGGGGTPLGLAGSGGSDIQLTQ |
| | | SPSSLSASVGDRVTITCRASQSIGSYLAWYQQKPGKAPKLLIYDASNLETGVPSR |
| | | FSGSGSGTDFTLTISSLQPEDFATYYCQQGYYLWTFGQGTKVEIKRTVAAPSV |
| | | FIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| | | (SEQ ID NO: 100) |
| TY23572 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKENWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 90) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 91) |
| | LC | EVGSYNDYNYHCKWRPSRCHNPLGLAGSGGSDIQLTQSPSSLSASVGDR |
| | | VTITCRASQSIGSYLAWYQQKPGKAPKLLIYDASNLETGVPSRFSGSGSGTDFT |
| | | LTISSLQPEDFATYYCQQGYYLWTFGQGTKVEIKRTVAAPSVFIFPPSDEQLK |
| | | SGTASVVCLLNNFYPREAKVQWKVDNALQSGNSQESVTEQDSKDSTYS |
| | | LSSTLTLSKADYEKHKVYACEVTHQGLSSPVTKSFNRGEC (SEQ ID NO: |
| | | 99) |
| TY25370 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKFNWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 94) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKFNWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 95) |
| | LC | EVGSYNDYNYHCKWRPSRCHNPLGLAGSGGSDIQLTQSPSSLSASVGDR |
| | | VTITCRASQSIGSYLAWYQQKPGKAPKLLIYDASNLETGVPSRFSGSGSGTDFT |
| | | LTISSLQPEDFATYYCQQGYYLWTFGQGTKVEIKRTVAAPSVFIFPPSDEQLK |
| | | SGTASVVCLLNNFYPREAKVQWKVDNALQSGNSQESVTEQDSKDSTYS |
| | | LSSTLTLSKADYEKHKVYACEVTHQGLSSPVTKSFNRGEC (SEQ ID NO: |
| | | 99) |
| TY23567 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYTDNYDYCPRLRRKCYHPLGLAGSGGSDIQLTQSPSSLSASVGDR |
| | | VTITCRASQSIGSYLAWYQQKPGKAPKLLIYDASNLETGVPSRFSGSGSGTDFT |
| | | LTISSLQPEDFATYYCQQGYYLWTFGQGTKVEIKRTVAAPSVFIFPPSDEQLK |
| | | SGTASVVCLLNNFYPREAKVQWKVDNALQSGNSQESVTEQDSKDSTYS |
| | | LSSTLTLSKADYEKHKVYACEVTHQGLSSPVTKSFNRGEC (SEQ ID NO: |
| | | 101) |
| TY25371 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKFNWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 94) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKENWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 95) |
| | LC | EVGSYTDNYDYCPRLRRKCYHPLGLAGSGGSDIQLTQSPSSLSASVGDR |
| | | VTITCRASQSIGSYLAWYQQKPGKAPKLLIYDASNLETGVPSRFSGSGSGTDFT |
| | | LTISSLQPEDFATYYCQQGYYLWTFGQGTKVEIKRTVAAPSVFIFPPSDEQLK |
| | | SGTASVVCLLNNFYPREAKVQWKVDNALQSGNSQESVTEQDSKDSTYS |
| | | LSSTLTLSKADYEKHKVYACEVTHQGLSSPVTKSFNRGEC (SEQ ID NO: |
| | | 101) |
| TY23570 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 90) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 91) |
| | LC | EVGSYTDNYDYCPRLRRKCYHSGRSAGGGGTPLGLAGSGGSDIQLTQ |
| | | SPSSLSASVGDRVTITCRASQSIGSYLAWYQQKPGKAPKLLIYDASNLETGVPSR |
| | | FSGSGSGTDFTLTISSLQPEDFATYYCQQGYYLWTFGQGTKVEIKRTVAAPSV |
| | | FIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| | | (SEQ ID NO: 102) |
| TY23573 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 90) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVSHEDPEVKFNWYVDGVEVHNAKTKPREEQYNST |
| | | YRVVSVLTVLHQDWLNGKEYKCKVSNKALPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 91) |
| | LC | EVGSYTDNYDYCPRLRRKCYHPLGLAGSGGSDIQLTQSPSSLSASVGDR |
| | | VTITCRASQSIGSYLAWYQQKPGKAPKLLIYDASNLETGVPSRFSGSGSGTDFT |
| | | LTISSLQPEDFATYYCQQGYYLWTFGQGTKVEIKRTVAAPSVFIFPPSDEQLK |
| | | SGTASVVCLLNNFYPREAKVQWKVDNALQSGNSQESVTEQDSKDSTYS |
| | | LSSTLTLSKADYEKHKVYACEVTHQGLSSPVTKSFNRGEC (SEQ ID NO: |
| | | 101) |
| TY25372 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKENWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 94) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKENWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 95) |
| | LC | EVGSYTDNYDYCPRLRRKCYHGGGPLGLAGGGSDIQLTQSPSSLSASVG |
| | | DRVTITCRASQSIGSYLAWYQQKPGKAPKLLIYDASNLETGVPSRFSGSGSGTD |
| | | FTLTISSLQPEDFATYYCQQGYYLWTFGQGTKVEIKRTVAAPSVFIFPPSDEQ |
| | | LKSGTASVVCLLNNFYPREAKVQWKVDNALQSGNSQESVTEQDSKDST |
| | | YSLSSTLTLSKADYEKHKVYACEVTHQGLSSPVTKSFNRGEC (SEQ ID |
| | | NO: 104) |
| TY25153 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYPTDLDACADAPNHCHFGGGPLGLAGGSDIQLTQSPSSLSASVGD |
| | | RVTITCRASQSIGSYLAWYQQKPGKAPKLLIYDASNLETGVPSRFSGSGSGTDF |
| | | TLTISSLQPEDFATYYCQQGYYLWTFGQGTKVEIKRTVAAPSVFIFPPSDEQL |
| | | KSGTASVVCLLNNFYPREAKVQWKVDNALQSGNSQESVTEQDSKDSTY |
| | | SLSSTLTLSKADYEKHKVYACEVTHQGLSSPVTKSFNRGEC (SEQ ID |
| | | NO: 105) |
| TY25154 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | LC | EVGSYPTDLDACADAPNHCHFSGGGPLGLAGGSDIQLTQSPSSLSASVG |
| | | DRVTITCRASQSIGSYLAWYQQKPGKAPKLLIYDASNLETGVPSRFSGSGSGTD |
| | | FTLTISSLQPEDFATYYCQQGYYLWTFGQGTKVEIKRTVAAPSVFIFPPSDEQ |
| | | LKSGTASVVCLLNNFYPREAKVQWKVDNALQSGNSQESVTEQDSKDST |
| | | YSLSSTLTLSKADYEKHKVYACEVTHQGLSSPVTKSFNRGEC (SEQ ID |
| | | NO: 106) |
| TY25155 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPCSRSTSESTAALGCLVKDYFP |
| | LC | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK (SEQ |
| | | ID NO: 88) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPCSRSTSESTAALGCLVKDYFP |
| | | EPVTVSWNSGALTSGVHTFPAVLQSSGLYSLSSVVTVPSSSLGTKTYTCN |
| | | VDHKPSNTKVDKRVESKYGPPCPPCPAPEFLGGPSVFLFPPKPKDTLMISR |
| | | TPEVTCVVVDVSQEDPEVQFNWYVDGVEVHNAKTKPREEQFNSTYRVV |
| | | SVLTVLHQDWLNGKEYKCKVSNKGLPSSIEKTISKAKGQPREPQVYTLPP |
| | | SQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLG (SEQ |
| | | ID NO: 89) |
| | | EVGSYPTDLDACADAPNHCHFSGRGPLGLAGGSDIQLTQSPSSLSASVG |
| | | DRVTITCRASQSIGSYLAWYQQKPGKAPKLLIYDASNLETGVPSRFSGSGSGTD |
| | | FTLTISSLQPEDFATYYCQQGYYLWTFGQGTKVEIKRTVAAPSVFIFPPSDEQ |
| | | LKSGTASVVCLLNNFYPREAKVQWKVDNALQSGNSQESVTEQDSKDST |
| | | YSLSSTLTLSKADYEKHKVYACEVTHQGLSSPVTKSFNRGEC (SEQ ID |
| | | NO: 107) |
| TY25369 | HC | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKFNWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPGK |
| | | (SEQ ID NO: 94) |
| | | EVQLVESGGGLVQPGGSLRLSCAASGFSLSTGGVGVGWIRQAPGKGLEWLAL |
| | | IDWADDKYYSPSLKSRLTISRDNSKNTLYLQLNSLRAEDTAVYYCARGGSDTVI |
| | | GDWFAYWGQGTLVTVSSTKGPSVFPLAPSSKSTSGGTAALGCLVKDYF |
| | | PEPVTVSWNSGALTSGVHTFPAVLQSSGLYSLSSVVTVPSSSLGTQTYICN |
| | | VNHKPSNTKVDKKVEPKSCDKTHTCPPCPAPELLGGPSVFLFPPKPKDTL |
| | | MISRTPEVTCVVVDVEHEDPEVKENWYVDGVEVHNAKTKPREEQYNST |
| | | YRVVSVLTVLHQDWLNGKEYKCKVSNKAFPAPIEKTISKAKGQPREPQV |
| | | YTLPPSRDELTKNQVSLTCLVKGFYPSDIAVEWESNGQPENNYKTTPPVL |
| | | DSDGSFFLYSKLTVDKSRWQQGNVFSCSVMHEALHNHYTQKSLSLSPG |
| | | (SEQ ID NO: 95) |
| | LC | EVGSYPTDLDACADAPNHCHFSGRGGGPLGLAGGSDIQLTQSPSSLSAS |
| | | VGDRVTITCRASQSIGSYLAWYQQKPGKAPKLLIYDASNLETGVPSRFSGSGSG |
| | | TDFTLTISSLQPEDFATYYCQQGYYLWTFGQGTKVEIKRTVAAPSVFIFPPSD |
| | | EQLKSGTASVVCLLNNFYPREAKVQWKVDNALQSGNSQESVTEQDSKD |
| | | STYSLSSTLTLSKADYEKHKVYACEVTHQGLSSPVTKSFNRGEC (SEQ |
| | | ID NO: 122) |

For LC, N-terminal unit is in plain text, masking unit is in underlined bold text, linkage unit is in bold text, VL sequence is in italicized text, CL is in double underlined text
For HC, VH sequence is in italicized text, CH is in double underlined text

In some embodiments, the term “masked anti-CD137 antibody” refers to an antibody fragment, e.g., a masked antigen-binding fragment of a masked anti-CD137 antibody. In some embodiments, the antibody fragment is or comprises a Fab, a Fab′, a Fab′-SH, a F(ab′)2, an Fv, an scFv (see Bird et al. (1988) Science 242:423-426 and Huston et al. (1988) Proc. Natl. Acad. Sci. USA 85:5879-5883), an (scFv)2, a linear antibody, a single-chain antibody, single domain antibody (nanobody) VHH, a minibody, or a diabody.

In some embodiments, a masked anti-CD137 antibody described herein cross-reacts with CD137 from different species, thus permitting the masked anti-CD137 antibody to be used in both preclinical and clinical studies. In some embodiments, a masked anti-CD137 antibody described herein binds to two or more of human CD137, cynomolgus CD137, murine (mouse) CD137, and/or rat CD137 following activation (i.e., after activation of the masked antibody via cleavage, e.g., protease cleavage). In some embodiments, a masked anti-CD137 antibody binds human CD137, cynomolgus CD137, murine (mouse) CD137, and a rat CD137 following activation (i.e., after activation of the masked antibody via cleavage, e.g., protease cleavage).

In some embodiments, masked anti-CD137 antibodies described herein are context-dependent (e.g., are activated (are only capable of binding their targets) in certain contexts (such as in the protease-rich tumor microenvironment)). In some embodiments, the masked anti-CD137 antibodies described herein provide improved safety over more traditional, non-masked antibodies (e.g., show reduced toxicity, do not induce significant alterations to the weights of many organs, do not alter liver histopathology, hematology, and/or blood biochemistry, etc.). In some embodiments, masked anti-CD137 antibodies described herein exhibit pharmacokinetic properties that are similar to those of traditional, non-masked anti-CD137 antibodies (e.g., have similar in vivo half-lives). In some embodiments, masked anti-CD137 antibodies described herein exhibit improved pharmacokinetic properties as compared to more traditional, non-masked anti-CD137 antibodies (e.g., have longer in vivo half-lives).

In some embodiments, the antibody heavy chain variable region (VH) and the antibody light chain variable region (VL) of a masked anti-CD137 antibody described herein form an antigen binding domain (ABD) that binds hCD137. In some embodiments, the masking unit (MU) of a masked anti-CD137 antibody described herein binds to the ABD of the and reduces or inhibits binding of the masked anti-CD137 antibody to hCD137, as compared to the binding of a corresponding anti-CD137 antibody lacking the MU to hCD137 and/or as compared to the binding of the ABD to hCD137. In some embodiments, the masking unit (MU) has a masking efficiency of at least about 2.0 (e.g., at least about 2.0, at least about 3.0, at least about 4.0, at least about 5.0, at least about 6.0, at least about 7.0, at least about 8.0, at least about 9.0, at least about 10, at least about 25, at least about 50, at least about 75, at least about 100, at least about 150, at least about 200, at least about 300, at least about 400, at least about 500, at least about 600, at least about 700, at least about 800, at least about 900, at least about 1,000, at least about 1,100, at least about 1,200, at least about 1,300, at least about 1,400, at least about 1,500, etc., including any range in between these values) prior to removing the MU from the masked anti-CD137 antibody. In some embodiments, masking efficiency is measured as the difference in affinity of the masked anti-CD137 antibody comprising the masking unit (MU) for binding to hCD137 (i.e., before activation of the masked antibody) relative to the affinity of an anti-CD137 antibody lacking the MU for binding to hCD137. In some embodiments, masking efficiency is measured as the difference in affinity for hCD137 of a masked anti-CD137 antibody comprising a MU (i.e., before activation of the masked antibody via cleavage, e.g., protease cleavage) relative to the affinity for hCD137 of the unmasked anti-CD137 antibody (i.e., after activation of the masked antibody via cleavage, e.g., protease cleavage). In some embodiments, the masking efficiency is measured by dividing the EC₅₀ for binding of a masked anti-CD137 antibody comprising an MU (i.e., before activation) by the EC₅₀ of a corresponding anti-CD137 antibody lacking the masking peptide or masking unit. In some embodiments, the EC₅₀ is measured by ELISA. In some embodiments, the masking unit (MU) of the masked anti-CD137 antibody binds to the ABD and prevents the masked anti-CD137 polypeptide from binding to hCD137.

In some embodiments, the affinity of a masked anti-CD137 antibody of the present disclosure increases by at least about 2-fold (e.g., at least about 2-fold, at least about 2.5-fold, at least about 3, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 25-fold, at least about 50-fold, at least about 75-fold, at least about 100-fold, at least about 250-fold, at least about 500-fold, at least about 750-fold, or at least about 1000-fold, or more, including any range in between the preceding values) when the masking unit is removed from the antibody (e.g., after activation by treatment with one or more proteases that cleave within the linkage unit) as compared to a corresponding anti-CD137 antibody without the masking peptide or masking unit. In some embodiments, the EC₅₀ of a masked anti-CD137 antibody described herein decreases by at least about 2-fold (e.g., at least about 2-fold, at least about 2.5-fold, at least about 3, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 25-fold, at least about 50-fold, at least about 75-fold, at least about 100-fold, at least about 250-fold, at least about 500-fold, at least about 750-fold, or at least about 1000-fold, or more, including any range in between the preceding values) after activation by treatment with one or more proteases that cleave within the linkage unit (e.g., as measured by an ELISA or FACS assay).

In some embodiments, when the masking unit is bound to the ABD of a masked anti-CD137 antibody described herein, the K_{D} of the antibody for its target is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more, including any range in between the preceding values) times greater than the K_{D} of the antibody when the masking unit of the masked anti-CD137 antibody is removed from the ABD (such as after protease treatment to cleave within the linkage unit). In some embodiments, when the masking unit is bound to the ABD of a masked anti-CD137 antibody described herein, the K_{D} of the antibody for its target is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more, including any range in between the preceding values) times greater than the K_{D} of a corresponding anti-CD137 antibody that lacks a masking peptide or masking unit.

In some embodiments, the masking unit sterically hinders binding of the masked binding polypeptide to its target and/or allosterically hinders binding of the masked binding polypeptide to its target.

In some embodiments, the dissociation constant of the masking unit for the ABD of a masked anti-CD137 antibody described herein is greater than the dissociation constant for the masked anti-CD137 antibody for hCD137 (when the masked anti-CD137 antibody is in active form, such as after protease treatment). In some embodiments, the dissociation constant of the masking unit for the ABD of a masked anti-CD137 antibody described herein is about 2 (e.g., about 2, about 2.5, about 3, about 3.5 about 4, about 4.5, about 5, about 5.5, about 6, about 6.5, about 7, about 7.5, about 8, about 8.5, about 9, about 9.5, about 10, about 25, about 50, about 75, about 100, about 250, about 500, about 750, or about 1000 or more, including any range in between the preceding values) times greater than the dissociation constant for the masked anti-CD137 antibody for hCD137 (when the masked anti-CD137 antibody is in active form, such as after protease treatment). In some embodiments, the dissociation constant of the masking unit for the ABD of a masked anti-CD137 antibody described herein is about equal to the dissociation constant for the masked anti-CD137 antibody for hCD137 (when the masked anti-CD137 antibody is in active form, such as after protease treatment). In some embodiments, the masking unit (MU) binds to the ABD of a masked anti-CD137 antibody described herein and prevents the antibody from binding to hCD137 only when the masked anti-CD137 antibody has not been activated (e.g., by treatment with one or more proteases that cleave within the linkage unit). In some embodiments, activation induces cleavage of the polypeptide within the cleavage site. In some embodiments, activation induces conformation changes in the polypeptide (e.g., displacement of the masking unit (MU)), leading to the masking peptide no longer preventing the polypeptide from binding to its target.

The masked antibodies described herein may be further modified. In some embodiments, the masked antibodies are linked to an additional molecular entity. Examples of additional molecular entities include pharmaceutical agents, peptides or proteins, detection agent or labels, and antibodies.

In some embodiments, an activatable binding polypeptide of the present disclosure is linked to a pharmaceutical agent. Examples of pharmaceutical agents include cytotoxic agents or other cancer therapeutic agents, and radioactive isotopes. Specific examples of cytotoxic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Methods for linking a polypeptide to a pharmaceutical agent are known in the art, such as using various linker technologies. Examples of linker types include hydrazones, thioethers, esters, disulfides, and peptide-containing linkers. For further discussion of linkers and methods for linking therapeutic agents to antibodies see e.g., Saito et al., Adv. Drug Deliv. Rev. 55:199-215 (2003); Trail, et al., Cancer Immunol. Immunother. 52:328-337 (2003); Payne, Cancer Cell 3:207-212 (2003); Allen, Nat. Rev. Cancer 2:750-763 (2002); Pastan and Kreitman, Curr. Opin. Investig. Drugs 3:1089-1091 (2002); Senter and Springer (2001) Adv. Drug Deliv. Rev. 53:247-264.

### Nucleic Acids, Vectors, Host Cells, and Recombinant Methods of Producing Methods of Producing Masked Anti-CD137 Antibodies

Another aspect of the disclosure provides one or more isolated nucleic acid molecule(s) that comprises nucleotide sequence(s) encoding an amino acid sequence(s) of a masked anti-CD137 antibody described herein. The amino acid sequence encoded by the nucleotide sequence may be any portion of a masked anti-CD137 antibody, such as a CDR, a sequence comprising one, two, or three CDRs, a variable region of a heavy chain, variable region of a light chain, or may be a full-length heavy chain or full length light chain. A nucleic acid of the disclosure can be, for example, DNA or RNA, and may or may not contain intronic sequences. Typically, the nucleic acid is a cDNA molecule.

In some embodiments, the disclosure provides an isolated nucleic acid molecule that comprises or consists of a nucleotide sequence encoding an amino acid sequence of, e.g., a heavy chain variable region and/or a light chain variable region of a masked anti-CD137 antibody described herein, or, e.g., a full length heavy chain and/or full length light chain of a masked anti-CD137 antibody described herein.

Nucleic acids of the disclosure can be obtained using any suitable molecular biology techniques, e.g., PCR amplification or cDNA cloning techniques. For masked anti-CD137 antibodies obtained via library screening, the nucleic acid encoding the antibody can be recovered from the library.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG4 or IgG2 constant region without ADCC effect. The IgG4 constant region sequence can be any of the various alleles or allotypes known to occur among different individuals. These allotypes represent naturally occurring amino acid substitution in the IgG4 constant regions. For a Fab fragment heavy chain gene, the V_{H}-encoding DNA can be operatively linked to another DNA molecule encoding only the heavy chain CHI constant region.

The isolated DNA encoding the V_{L} region can be converted to a full-length light chain gene (as well as a Fab light chain gene) by operatively linking the V_{L}-encoding DNA to another DNA molecule encoding the light chain constant region, CL. The sequences of human light chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region. In some embodiments, the masked anti-CD137 antibody comprises a light chain constant region set forth in SEQ ID NO: 111, which is provided below:

(SEQ ID NO: 111)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

To create a scFv gene, the V_{H}- and V_{L}-encoding DNA fragments are operatively linked to another fragment encoding a flexible linker, e.g., encoding the amino acid sequence (Gly₄-Ser)₃ (SEQ ID NO: 128), such that the V_{H} and V_{L} sequences can be expressed as a contiguous single-chain protein, with the V_{L} and V_{H} regions joined by the flexible linker (see e.g., Bird et al., Science 242:423-426 (1988); Huston et al., Proc. Natl. Acad. Sci. USA 85:5879-5883 (1988); and McCafferty et al., Nature 348:552-554 (1990)).

The present disclosure further provides a vector that comprises one or more nucleic acid molecule(s) provided by the present disclosure. In some embodiments, the vector is an expression vector useful for the expression of a masked anti-CD137 antibody or a masked antigen binding fragment of such an antibody. In some embodiments, provided herein are vectors, wherein a first vector comprises a polynucleotide sequence encoding a heavy chain variable region as described herein, and a second vector comprises a polynucleotide sequence encoding a light chain variable region as described herein. In some embodiments, a single vector comprises polynucleotides encoding a heavy chain variable region as described herein and a light chain variable region as described herein.

To express a binding molecule of the disclosure, DNAs encoding partial or full-length light and heavy chains are inserted into expression vectors such that the DNA molecules are operatively linked to transcriptional and translational control sequences. In this context, the term “operatively linked” means that an antibody gene is ligated into a vector such that transcriptional and translational control sequences within the vector serve their intended function of regulating the transcription and translation of the DNA molecule. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by any suitable methods (e.g., ligation of complementary restriction sites on the antibody gene fragment and vector, or homologous recombination-based DNA ligation). The light and heavy chain variable regions of the antibodies described herein can be used to create full-length antibody genes of any antibody isotype and subclass by inserting them into expression vectors already encoding heavy chain constant and light chain constant regions of the desired isotype and subclass such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the expression vectors of the disclosure typically carry regulatory sequences that control the expression of the antibody chain genes in a host cell. The term “regulatory sequence” is intended to include promoters, enhancers, and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Examples of regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SR promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the expression vectors may carry additional sequences, such as enhancer element(s), a transcription termination sequence(s), sequence(s) that regulate replication of the vector in host cells (e.g., origins of replication) and selectable marker gene(s). The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by any suitable techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the masked anti-CD137 antibodies described herein in either prokaryotic or eukaryotic host cells, expression of antibodies in eukaryotic cells, e.g., mammalian host cells, is most typical.

The present disclosure further provides a host cell containing nucleic acid molecule(s) or vector(s) provided by the present disclosure. The host cell can be virtually any cell for which expression vectors are available. It may be, for example, a higher eukaryotic host cell, such as a mammalian cell, a lower eukaryotic host cell, such as a yeast cell, and may be a prokaryotic cell, such as a bacterial cell. Introduction of the recombinant nucleic acid construct into the host cell can be effected by calcium phosphate transfection, DEAE, dextran mediated transfection, electroporation, or phage infection.

Suitable prokaryotic hosts for transformation include E. coli, Bacillus subtilis, Salmonella typhimurium and various species within the genera Pseudomonas, Streptomyces, and Staphylococcus.

Mammalian host cells for expressing a binding molecule of the disclosure include, for example, Chinese Hamster Ovary (CHO) cells (including dhfr-CHO cells, described in Urlaub and Chasin, Proc. Natl. Acad. Sci. USA 77:4216-4220 (1980), used with a DHFR selectable marker, e.g., as described in Kaufman and Sharp, J. Mol. Biol. 159:601-621 (1982), NSO myeloma cells, COS cells and Sp2 cells. In particular, for use with NSO myeloma or CHO cells, another expression system is the GS (glutamine synthetase) gene expression system disclosed in WO 87/04462, WO 89/01036, and EP 338,841.

A masked anti-CD137 antibody (or antigen binding fragment thereof) of the present disclosure may be produced by any means known in the art. Exemplary techniques for antibody production are in U.S. Pat. No. 4,816,567; however these exemplary techniques are provided for illustrative purposes only and are not intended to be limiting. When nucleic acid(s) or expression vector(s) encoding a masked anti-CD137 antibody are introduced into a host cell, the masked anti-CD137 antibody is produced by culturing the host cell for a period of time sufficient to allow for expression of the antibody in the host cells or secretion of the antibody into the culture medium in which the host cells are grown. Thus, in some embodiments, provided is a method of producing a masked anti-CD137 antibody described herein, which method comprises culturing a host cell comprising one or more nucleic acid(s) or vector(s) that encode the masked anti-CD137 antibody (e.g., as provided above) under conditions suitable for expression of the masked antibody. In some embodiments, the method further comprises recovering the masked anti-CD137 antibody from the host cell (or host cell culture medium). The masked anti-CD137 antibody can be recovered from the culture medium using any suitable protein purification methods.

### Pharmaceutical Compositions

In other aspects, the present disclosure provides a composition comprising one or more masked anti-CD137 antibodies described herein. In some embodiments, the composition is a pharmaceutical composition comprising masked anti-CD137 antibody described herein and a pharmaceutically acceptable carrier. The compositions can be prepared by conventional methods known in the art.

The term “pharmaceutically acceptable carrier” refers to any inactive substance that is suitable for use in a formulation for the delivery of a polypeptide (e.g., a masked antibody). A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering a masked anti-CD137 antibody is a sterile liquid, such as a solution, suspension, or dispersion, for injection or infusion. Sterile solutions can be prepared by incorporating the masked anti-CD137 antibody in the required amount in an appropriate carrier, followed by sterilization microfiltration. Dispersions may be prepared by incorporating the masked anti-CD137 antibody into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of a masked anti-CD137 antibody included in the composition will vary depending upon a number of factors, such as the specific polypeptide and carriers used, dosage form, and desired release and pharmacodynamic characteristics. The amount of a masked anti-CD137 antibody in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In addition to the masked anti-CD137 antibody, one or more additional therapeutic agents may be included in the composition. Examples of additional therapeutic agents are described in WO 2019/037711, the contents of which are incorporated herein by reference in their entirety. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent which produces a therapeutic effect, but may be a lesser amount as well.

Any of the masked anti-CD137 antibody) and/or compositions (e.g., pharmaceutical compositions) described herein may be used in the preparation of a medicament (e.g., a medicament for use in treating or delaying progression of cancer in a subject in need thereof).

### Methods of Treatment

The masked anti-CD137 antibodies and pharmaceutical compositions described herein are useful for therapeutic purposes, such as treating cancer or enhancing the efficacy of other cancer therap(ies). Thus, in other aspects, the present disclosure provides methods of using the masked anti-CD137 antibodies or pharmaceutical compositions. In one aspect, the present disclosure provides a method of treating cancer in a subject (e.g., a human subject), comprising administering to the subject an effective amount of a masked anti-CD137 antibody. In some embodiments, the cancer is solid tumor cancer (e.g., breast cancer, liver cancer, colorectal cancer, colon cancer etc.).

In practicing the therapeutic methods, the masked anti-CD137 antibodies described herein may be administered alone, i.e., as monotherapy, or administered in combination with one or more additional therapeutic agents or therapies. Thus, in another aspect, the present disclosure provides a combination therapy, which comprises a binding molecule in combination with one or more additional therapies or therapeutic agents for separate, sequential, or simultaneous administration. In some embodiments, the term “additional therapy” refers to a therapy which does not employ a masked anti-CD137 antibody as a therapeutic agent. In some embodiments, the term “additional therapeutic agent” refers to any therapeutic agent other than a masked anti-CD137 antibody described herein. In some embodiments, the present disclosure provides a method of treating cancer in a subject (e.g., a human subject) that comprises administering to the subject an effective amount of a masked anti-CD137 antibody described herein and an effective amount of an anti-PD-1 antibody (e.g., an anti-human PD-1 antibody). In some embodiments, the present disclosure provides a method of treating cancer in a subject (e.g., a human subject) that comprises administering to the subject an effective amount of a masked anti-CD137 antibody described herein and an effective amount of an anti-CTLA4 antibody (e.g., an anti-human CTLA4 antibody). In some embodiments, the anti-CTLA4 antibody is a masked anti-CTLA4 antibody. In some embodiments, the present disclosure provides a method of treating cancer in a subject (e.g., a human subject) that comprises administering to the subject an effective amount of a masked anti-CD137 antibody described herein and an effective amount of a bispecific antibody that binds HER2 (e.g., human HER2) and CD3 (e.g., human CD3). In some embodiments, the bispecific antibody that binds HER2 and CD3 is a masked bispecific antibody that binds HER2 and CD3. In some embodiments, the present disclosure provides a method of treating cancer in a subject (e.g., a human subject) that comprises administering to the subject an effective amount of a masked anti-CD137 antibody described herein and an effective amount of a bispecific T cell engager (TCE) that targets CD3 (e.g., human CD3) and an antigen expressed on the surface of a solid tumor cancer cell. In some embodiments, the antigen expressed on the surface of the solid tumor is HER2 (e.g., human HER2). In some embodiments, the TCE that targets CD3 and HER2 comprises three polypeptide chains, wherein a first polypeptide chain comprises an antibody heavy chain, a second polypeptide chain comprises an antibody light chain, and a third polypeptide chain comprises an scFv-Fc domain fusion wherein the C-terminus of the ScFv is fused to the N-terminus of the Fc domain, wherein the first and second polypeptide chains associate to from a HER2 binding arm, wherein the third polypeptide chain binds CD3, and wherein an Fc domain of the anti-HER2 binding arm and the Fc of the third polypeptide chain dimerize to form the TCE. In some embodiments, the TCE is a masked TCE (e.g., wherein the portion of the TCE that binds CD3 and/or the portion of the TCE that binds HER2 are masked). In some embodiments, the first polypeptide chain comprises SEQ ID NO: 125, the second polypeptide chain comprises SEQ ID NO: 124, and the third polypeptide chain comprises SEQ ID NO: 126. In some embodiments, the cancer is solid tumor (e.g., colon cancer, breast cancer, liver cancer, colorectal cancer, or colon cancer).

In some embodiments of any of the methods of treatment described herein, the masked anti-CD137 antibody comprises the masking peptide of SEQ ID NO: 34, a VH domain set forth in SEQ ID NO: 52, and a VL domain set forth in SEQ ID NO: 53. In some embodiments, the masked anti-CD137 antibody further comprise a human IgG1 domain or a variant thereof that comprises one or more substitution mutation(s). In some embodiments the IgGI variant comprises substitution(s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index. In some embodiments, the masked anti-CD137 antibody further comprise a human IgG4 domain or a variant thereof that comprises one or more substitution mutation(s). In some embodiments the IgG4 variant comprises substitution(s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D. and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index.

### Kits and Articles of Manufacture

In another aspect, provided is a kit comprising one or more masked anti-CD137 antibodies described herein. In some embodiments, the kit further comprises a package insert comprising instructions for use of the masked anti-CD137 antibodies. In some embodiments, the article of manufacture or kit comprises a container containing one or more of the masked anti-CD137 antibodies or compositions described herein. In certain embodiments, the article of manufacture or kit comprises a container containing nucleic acid(s) encoding one (or more) of the masked anti-CD137 antibodies described herein. In some embodiments, the kit includes a cell of cell line that produces a masked anti-CD137 antibody described herein. In some embodiments, the kit includes one or more positive controls, for CD137 (e.g., human CD137, cynomolgus CD137, mouse CD137, rat CD137 or fragments of any of the preceding) or CD137⁺ cells. In some embodiments, the kit includes negative controls, for example a surface or solution that is substantially free of CD137, or a cell that does not express CD137.

In certain embodiments, the article of manufacture or kit comprises a container and a label or package insert on or associated with the container. In some embodiments, the label or package insert indicates that the masked anti-CD137 antibody is for use in the treatment of solid tumor (e.g., breast cancer, liver cancer, colorectal cancer, colon cancer, etc.) in a subject (e.g., human subject) in need thereof, e.g., according to a method provided herein. Suitable containers include, for example, bottles, vials, syringes, IV solution bags, test tubes, etc. The containers may be formed from a variety of materials such as glass or plastic. The container holds a masked anti-CD137 antibody (or a composition comprising such masked antibody), which is by itself or combined with another composition effective for treating, delaying progression of, and/or preventing cancer in a subject (e.g. a human subject). The container may have a sterile access port (for example the container may be an intravenous solution bag or a vial having a stopper pierceable by a hypodermic injection needle). In some embodiments, the label or package insert indicates that the composition is used for treating breast cancer, liver cancer, colorectal cancer, or colon cancer in a subject (e.g., a human subject).

Moreover, the article of manufacture or kit may comprise (a) a first container with a composition contained therein, wherein the composition comprises a masked anti-CD137 antibody (or immunologically active fragment thereof) described herein; and (b) a second container with a composition contained therein, wherein the composition comprises a further cytotoxic or otherwise therapeutic agent. In some embodiments, the second container contains a composition comprising an anti-PD-1 antibody (e.g., an anti-human PD-1 antibody), and the article of manufacture comprises a label or package insert indicates that the masked anti-CD137 antibody and the anti-PD-L1 are for use in the treatment of solid tumor (e.g., breast cancer, liver cancer, colorectal cancer, colon cancer, etc.) in a subject (e.g., human subject) in need thereof, e.g., according to a method provided herein. In some embodiments, the second container contains a composition comprising an anti-CTLA4 antibody (e.g., an anti-human CTLA4 antibody, such as a masked anti-CTLA4 antibody), and the article of manufacture comprises a the label or package insert indicates that the masked anti-CD137 antibody and the anti-CTLA4 antibody (e.g., a masked anti-CTLA4 antibody) are for use in the treatment of solid tumor (e.g., breast cancer, liver cancer, colorectal cancer, colon cancer, etc.) in a subject (e.g., human subject) in need thereof, e.g., according to a method provided herein. In some embodiments, the second container contains a composition comprising a bispecific antibody that binds HER2 (e.g., human HER2) and CD3 (e.g., human CD3), such as a masked bispecific antibody that binds HER2 and CD3, and the article of manufacture comprises a the label or package insert indicates that the masked anti-CD137 antibody and the bispecific antibody that binds HER2 and CD3 (e.g., a masked bispecific antibody that binds HER2 and CD3) are for use in the treatment of solid tumor (e.g., breast cancer, liver cancer, colorectal cancer, colon cancer, etc.) in a subject (e.g., human subject) in need thereof, e.g., according to a method provided herein. In some embodiments, the second container contains a composition comprising a bispecific T-cell engager (TCE) that binds CD3 (e.g., human CD3) and an antigen expressed on the surface of a solid tumor cancer cell (e.g., a masked bispecific T-cell engager that binds CD3 and an antigen expressed on the surface of a solid tumor cancer cell). In some embodiments, the antigen expressed on the surface of the solid tumor is HER2 (e.g., human HER2). In some embodiments, the TCE that targets CD3 and HER2 comprises three polypeptide chains, wherein a first polypeptide chain comprises an antibody heavy chain, a second polypeptide chain comprises an antibody light chain, and a third polypeptide chain comprises an scFv-Fc domain fusion wherein the C-terminus of the ScFv is fused to the N-terminus of the Fc domain, wherein the first and second polypeptide chains associate to from a HER-2 binding arm, wherein the third polypeptide chain binds CD3, and wherein the Fc domain of the anti-HER2 binding arm and the Fc of the third polypeptide chain dimerize to form the TCE. In some embodiments, the TCE is a masked TCE (e.g., wherein the portion of the TCE that binds CD3 and/or the portion of the TCE that binds HER2 are masked). In some embodiments, the first polypeptide chain comprises SEQ ID NO: 125, the second polypeptide chain comprises SEQ ID NO: 124, and the third polypeptide chain comprises SEQ ID NO: 126. In some embodiments, the article of manufacture comprises a the label or package insert indicates that the masked anti-CD137 antibody and the TCE that binds CD3 and an antigen expressed on the surface of a solid tumor cancer cell (e.g., a masked bispecific T-cell engager that binds CD3 and an antigen expressed on the surface of a solid tumor cancer cell, such as HER2) are for use in the treatment of solid tumor (e.g., breast cancer, liver cancer, colorectal cancer, colon cancer, etc.) in a subject (e.g., human subject) in need thereof, e.g., according to a method provided herein.

Additionally, the article of manufacture may further comprise an additional container comprising a pharmaceutically acceptable buffer, such as bacteriostatic water for injection (BWFI), phosphate-buffered saline, Ringer's solution, and dextrose solution. It may further include other materials desirable from a commercial and user standpoint, including other buffers, diluents, filters, needles, and syringes.

The foregoing written description is considered to be sufficient to enable one skilled in the art to practice the present disclosure. The following Examples are offered for illustrative purposes only, and are not intended to limit the scope of the present disclosure in any way. Indeed, various modifications of the present disclosure in addition to those shown and described herein will become apparent to those skilled in the art from the foregoing description and fall within the scope of the appended claims.

All references cited herein, including patent applications, patent publications, and UniProtKB/Swiss-Prot Accession numbers are herein incorporated by reference in their entirety, as if each individual reference were specifically and individually indicated to be incorporated by reference.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Construction and Validation of Masked Antibodies Targeting CD137

### FACS-Based Screening of Masking Peptides Against a CD137 Antibody

A total of 1×10⁸ yeast cells from a constrained peptide library (CPL) library described in US 2019/00241886 (the contents of which are incorporated herein by reference in their entirety) were used to screen for masking peptides against parental anti-CD137 antibody TY21242, which comprises the heavy chain variable domain (VH) set forth in SEQ ID NO: 52 and light chain variable domain (VL) sequence set forth in SEQ OD NO: 53, which are provided below. (CDR sequences are in bold type).

(SEQ ID NO: 52)
EVQLVESGGGLVQPGGSLRLSCAASGFSLSTGGVGVGWIRQAPGKGLEWL
ALIDWADDKYYSPSLKSRLTISRDNSKNTLYLQLNSLRAEDTAVYYCARG
GSDTVIGDWFAYWGQGTLVTVSS
(SEQ ID NO: 53)
DIQLTQSPSSLSASVGDRVTITCRASQSIGSYLAWYQQKPGKAPKLLIYD
ASNLETGVPSRFSGSGSGTDFTLTISSLQPEDFATYYCQQGYYLWTFGQG
TKVEIKR

In this example, the masking units from the CPL were each directly fused to the N-terminus of the light chain of the parental antigen binding domain, and a yeast library was constructed that displayed the fusion proteins on the yeast cell surface. To identify the masking peptide sequences that can effectively mask the parental anti-CD137 antibody, the yeast library then underwent several rounds of FACS-based screening: first the yeast clones that had low binding to human CD137 were enriched, then the enriched yeast clones were treated with a protease to remove the masking unit, and the clones with high binding to antigen were selected.

Briefly, for each round of sorting through a MOFLO XDP® high speed cell sorter, yeast cells induced in galactose medium were harvested, washed once with PBSA buffer (i.e., 1% BSA in PBS), and then incubated with different concentrations of biotinylated CD137 for 1 hour at room temperature. The yeast cells were then washed twice with PBSA buffer, and incubated with phycoerythrin (PE) conjugated streptavidin (1:500 dilution) (eBioscience #2-4317-87) for 30 minutes at 4° C. After two more washes with PBSA buffer, the yeast cells were adjusted to 2-3 OD/mL, and subject to sorting. 1 nM of biotinylated CD137-Fc was used in all 5 rounds of sorting, and the weak binders were enriched in each round. The yeast cells displaying weak binders from round 5 were grown in glucose and were then induced in galactose medium and treated with TEV protease (6U/OD cell) (in-house) for 2 hours at 30° C. Yeast cells that displayed strong binders following protease treatment were purified. It was apparent that TEV cleavage resulted in a dramatic increase of the population of cells that bound strongly to antigen, suggesting that the screening strategy was effective. The single clones from the 5th round of sorting were plated on selective media and grown individually for further confirmation of cleavage mediated activated antigen binding. In Fab format, the selected masked anti-CD137 antibody clones, exhibited little binding to antigen (i.e., hCD137) in the presence of masking peptide. However, binding to antigen was dramatically increased when the yeast cells were treated with TEV protease to remove the masking peptide.

After 5 rounds of sorting, the plasmids were extracted from these clones and the masking unit sequences were confirmed through DNA sequencing. To determine the amino acid sequences of the masking peptides, the shuttle plasmids were extracted from the selected yeast clones (Generay #GK2002-200) and transformed into competent E. coli cells. The plasmids were prepared, and the regions encoding the masking peptides were sequenced and aligned. The sequences of the masking peptides could be separated into several groups, indicating clear enrichment through rounds of sorting. The masking unit sequences and linkage unit sequences for several antibodies are listed in Table A1. The complete sequences of the masking peptides are shown in Table A2. The masking peptides comprise, from N-terminus to C-terminus, an N-terminal unit (i.e., the amino acid sequence EVGSY (SEQ ID NO: 77)), a masking unit, and a linkage unit. The VH and VL sequences of the antibodies listed in Tables A1 and A2 are provided in Tables 5A and 5B.

TABLE A1
Masking unit sequences and linkage unit sequences

| | SEQ ID NO | Masking Unit sequences | SEQ ID NO | Linkage Unit sequences | IgG |
|---|---|---|---|---|---|
| Antibody | → | (N- to C-terminus) | → | (N- to C-terminus) | isotype |
| TY22586 | 1 | PTDLDACADAPNHCHF | 8 | SGRSAGGGGTPLGLAGSGGS | hIgG4 |
| TY22591 | 2 | SSTHAHCHHSPANCIS | 8 | SGRSAGGGGTPLGLAGSGGS | hIgG4 |
| TY22594 | 3 | DTDYDFCPILRHRCDS | 8 | SGRSAGGGGTPLGLAGSGGS | hIgG4 |
| TY22595 | 4 | NDYNYHCKWRPSRCHN | 8 | SGRSAGGGGTPLGLAGSGGS | hIgG4 |
| TY22596 | 5 | YHDYDDCRVLPRRCFN | 8 | SGRSAGGGGTPLGLAGSGGS | hIgG4 |
| TY22598 | 6 | SNNFASCLWRHRSCAD | 8 | SGRSAGGGGTPLGLAGSGGS | hIgG4 |
| TY22599 | 7 | TDNYDYCPRLRRKCYH | 8 | SGRSAGGGGTPLGLAGSGGS | hIgG4 |

TABLE A2
Masking peptides comprising N-terminal unit, masking unit and linkage unit
sequences*

| | SEQ ID NO | | IgG |
|---|---|---|---|
| Antibody | → | (N- to C-terminus) | isotype |
| TY22586 | 17 | EVGSYPTDLDACADAPNHCHFSGRSAGGGGTPLGLAGSGGS | hIgG4 |
| TY22591 | 18 | EVGSYSSTHAHCHHSPANCISSGRSAGGGGTPLGLAGSGGS | hIgG4 |
| TY22594 | 19 | EVGSYDTDYDFCPILRHRCDSSGRSAGGGGTPLGLAGSGGS | hIgG4 |
| TY22595 | 20 | EVGSYNDYNYHCKWRPSRCHNSGRSAGGGGTPLGLAGSGGS | hIgG4 |
| TY22596 | 21 | EVGSYYHDYDDCRVLPRRCFNSGRSAGGGGTPLGLAGSGGS | hIgG4 |
| TY22598 | 22 | EVGSYSNNFASCLWRHRSCADSGRSAGGGGTPLGLAGSGGS | hIgG4 |
| TY22599 | 23 | EVGSYTDNYDYCPRLRRKCYHSGRSAGGGGTPLGLAGSGGS | hIgG4 |

*N-terminal unit sequences are in plain type; masking sequences are in bold underlined type; linkage unit sequences are in bold type.

### IgG Conversion and Expression

The masking unit and linkage unit sequences of additional masked anti-CD137 antibodies derived from TY22586, TY22594, TY22595, and TY22599 are listed below in Table BL. The complete sequences of the masking peptides are shown in Table B2. The masking peptides comprise, from N-terminus to C-terminus, an N-terminal unit (i.e., the amino acid sequence EVGSY (SEQ ID NO: 77)), a masking unit, and a linkage unit. The VH and VL sequences of the antibodies in Tables B1 and B2 are provided in Tables 5A and 5B. The antibodies in Tables B1 and B2 were converted into IgG1 or IgG4 masked antibodies, and each masked antibody contains one or two cleavage sites (i.e., uPA and/or a matrix metalloproteinase (MMP), such as MMP9). To enhance the binding of the masked antibodies in Tables B1 and B2 to FcγRIIb, S267E and L328F mutations (EU numbering) were introduced into the Fc regions to generate TY25370, TY25371, TY25372, TY25366, TY25368 and TY25369. The heavy and light chains of each antibody were cloned separately into the mammalian expression vector pCDNA3.3 (Thermo Fisher Scientific, cat #K830001), and the masking peptides and the cleavage peptides were fused to the N-terminus of the light chain, i.e., in the same manner as displayed on yeast surfaces in the CPL.

TABLE B1
Masking unit and linkage unit sequences for masked anti-CD137 antibodies derived
from TY22586, TY22594, TY22595, and TY22599

| | SEQ ID | Masking Unit sequences | SEQ ID | Linkage Unit sequences | IgG | |
|---|---|---|---|---|---|---|
| Antibody | NO → | (N- to C-terminus) | NO → | (N- to C-terminus) | isotype | Fc mutations |
| TY23565 | 3 | DTDYDFCPILRHRCDS | 10 | PLGLAGSGGS | hIgG4 | |
| TY23568 | 3 | DTDYDFCPILRHRCDS | 8 | SGRSAGGGGTPLGLAGSGGS | hIgG1 | |
| TY23571 | 3 | DTDYDFCPILRHRCDS | 10 | PLGLAGSGGS | hIgG1 | |
| TY23566 | 4 | NDYNYHCKWRPSRCHN | 10 | PLGLAGSGGS | hIgG4 | |
| TY23569 | 4 | NDYNYHCKWRPSRCHN | 8 | SGRSAGGGGTPLGLAGSGGS | hIgG1 | |
| TY23572 | 4 | NDYNYHCKWRPSRCHN | 10 | PLGLAGSGGS | hIgG1 | |
| TY25370 | 4 | NDYNYHCKWRPSRCHN | 10 | PLGLAGSGGS | hIgG1 | S267E, L328F |
| TY23567 | 7 | TDNYDYCPRLRRKCYH | 10 | PLGLAGSGGS | hIgG4 | |
| TY25371 | 7 | TDNYDYCPRLRRKCYH | 10 | PLGLAGSGGS | hIgG1 | S267E, L328F |
| TY23570 | 7 | TDNYDYCPRLRRKCYH | 8 | SGRSAGGGGTPLGLAGSGGS | hIgG1 | |
| TY23573 | 7 | TDNYDYCPRLRRKCYH | 10 | PLGLAGSGGS | hIgG1 | |
| TY25372 | 7 | TDNYDYCPRLRRKCYH | 11 | GGGPLGLAGGGS | hIgG1 | S267E, L328F |
| TY25153 | 1 | PTDLDACADAPNHCHF | 12 | GGGPLGLAGGS | hIgG4 | |
| TY25154 | 1 | PTDLDACADAPNHCHF | 13 | SGGGPLGLAGGS | hIgG4 | |
| TY25155 | 1 | PTDLDACADAPNHCHF | 14 | SGRGPLGLAGGS | hIgG4 | |
| TY25366 | 1 | PTDLDACADAPNHCHF | 15 | SGRGGPLGLAGGS | hIgG4 | S267E, L328F |
| TY25368 | 1 | PTDLDACADAPNHCHF | 15 | SGRGGPLGLAGGS | hIgG1 | S267E, L328F |
| TY25369 | 1 | PTDLDACADAPNHCHF | 16 | SGRGGGPLGLAGGS | hIgG1 | S267E, L328F |

TABLE B2
Masking peptides comprising n-terminal unit, masking unit and linkage unit sequences
for masked anti-CD137 antibodies derived from TY22586, TY22594, TY22595, and TY22599

| | SEQ ID | Masking Peptide Sequences | IgG | |
|---|---|---|---|---|
| Antibody | NO → | (N- to C-terminus) | isotype | Fc mutation |
| TY23565 | 24 | EVGSYDTDYDFCPILRHRCDSPLGLAGSGGS | hIgG4 | |
| TY23568 | 25 | EVGSYDTDYDFCPILRHRCDSSGRSAGGGGTPLGLAGSGGS | hIgG1 | |
| TY23571 | 24 | EVGSYDTDYDFCPILRHRCDSPLGLAGSGGS | hIgG1 | |
| TY23566 | 26 | EVGSYNDYNYHCKWRPSRCHNPLGLAGSGGS | hIgG4 | |
| TY23569 | 27 | EVGSYNDYNYHCKWRPSRCHNSGRSAGGGGTPLGLAGSGGS | hIgG1 | |
| TY23572 | 26 | EVGSYNDYNYHCKWRPSRCHNPLGLAGSGGS | hIgG1 | |
| TY25370 | 26 | EVGSYNDYNYHCKWRPSRCHNPLGLAGSGGS | hIgG1 | S267E, L328F |
| TY23567 | 28 | EVGSYTDNYDYCPRLRRKCYHPLGLAGSGGS | hIgG4 | |
| TY25371 | 28 | EVGSYTDNYDYCPRLRRKCYHPLGLAGSGGS | hIgG1 | S267E, L328F |
| TY23570 | 29 | EVGSYTDNYDYCPRLRRKCYHSGRSAGGGGTPLGLAGSGGS | hIgG1 | |
| TY23573 | 28 | EVGSYTDNYDYCPRLRRKCYHPLGLAGSGGS | hIgG1 | |
| TY25372 | 30 | EVGSYTDNYDYCPRLRRKCYHGGGPLGLAGGGS | hIgG1 | S267E, L328F |
| TY25153 | 31 | EVGSYPTDLDACADAPNHCHFGGGPLGLAGGS | hIgG4 | |
| TY25154 | 32 | EVGSYPTDLDACADAPNHCHFSGGGPLGLAGGS | hIgG4 | |
| TY25155 | 33 | EVGSYPTDLDACADAPNHCHFSGRGPLGLAGGS | hIgG4 | |
| TY25366 | 34 | EVGSYPTDLDACADAPNHCHFSGRGGPLGLAGGS | hIgG4 | S267E, L328F |
| TY25368 | 34 | EVGSYPTDLDACADAPNHCHFSGRGGPLGLAGGS | hIgG1 | S267E, L328F |
| TY25369 | 35 | EVGSYPTDLDACADAPNHCHFSGRGGGPLGLAGGS | hIgG1 | S267E, L328F |

*N-terminal unit sequences are in plain type; masking sequences are in bold underlined type; linkage units are in bold type.

Plasmid pairs encoding the VH and VL of each masked anti-CD137 antibody (i.e., with N-terminus of each VL fused to peptide comprising masking sequences and linkage units) were transiently transfected into HEK293F cells. After six days, the supernatants were harvested, cleared by centrifugation and filtration, and antibodies were purified via standard protein A affinity chromatography (MabSelect SuRe, GE Healthcare). The masked anti-CD137 antibodies were eluted, neutralized, and buffer exchanged into 20 mM histidine, pH 5.5 buffer. Protein concentrations were determined by UV-spectrophotometry, and antibody purity was analyzed under denaturing, reducing, and non-reducing conditions via SDS-PAGE or SEC-HPLC. The expression levels of the masked anti-CD137 antibodies in HEK293 cells were similar to or lower than their parental antibody, and their purification yields after protein A resin were also similar to the parental antibody, suggesting that the presence of the masking and cleavage peptides do not have a significant negative impact on antibody expression in mammalian cells.

### Measurement of Masking Efficiency

The affinities of the masked anti-CD137 antibodies for human CD137 displayed on the surface of yeast cells were also assessed. Briefly, yeast cells were transformed with the plasmids expressing full-length human CD137 followed with c-terminal 3×Myc tag, which was used to identify the transformed cells. The transformed cells were transferred to 1.5 mL tube, washed once with 1% PBSA buffer, centrifuged, and resuspended with 1 mL 1% PBSA buffer to a density is 0.2 OD₆₀₀/mL, and aliquoted to wells in a 96 well plate. 3-fold serial dilutions of the test antibodies were pipetted into cell-containing wells of the 96-well plate, incubated on ice for 1 hour (protected from light), washed once with 1% PBSA buffer, and incubated with 0.5 pg/mL PE-conjugated mouse anti-human IgG Fc and 2 μg/mL mouse anti-myc-647 for 30 min on ice. The cells were washed once prior to analysis by flow cytometry (Beckman® CytoFlex).

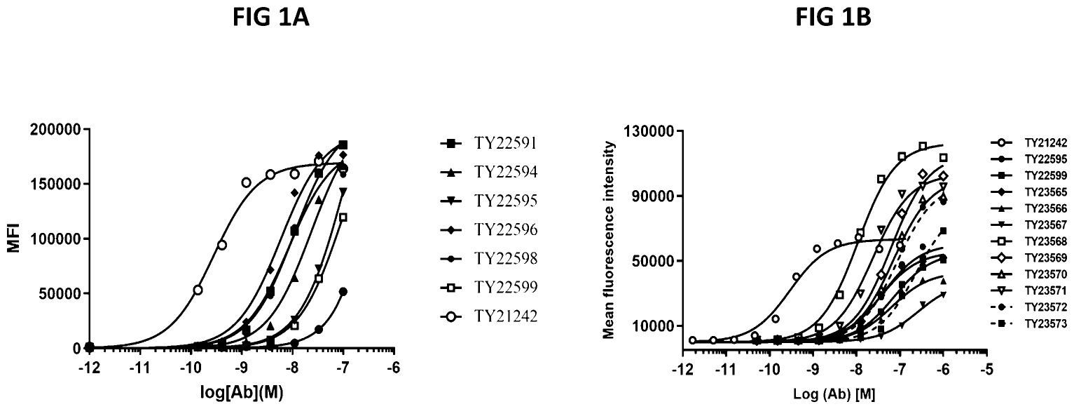

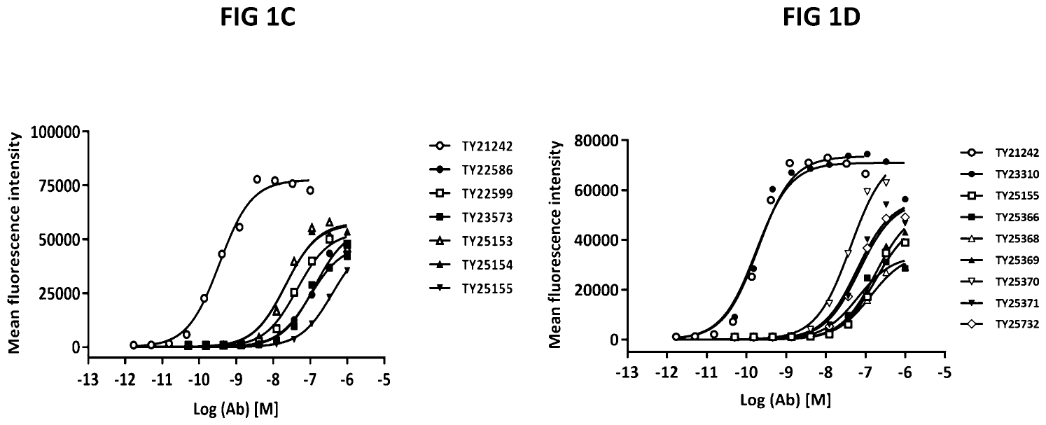

Experiments for some masked anti-CD137 antibodies were performed multiple times, leading to two calculated masking efficiencies being obtained for each of these masked antibodies. Masking efficiencies for each masked antibody were calculated by dividing the KD for binding of the masked antibody to hCD137 by the KD of the parental antibody to hCD137. As shown in FIGS. 1A-1D and Table C below, each of the masked antibodies showed dramatically reduced binding to hCD137 as compared with the parental antibody. The calculated masking efficiencies of the masked antibodies ranged from 21 to 1131. Differences in masking efficiencies likely resulted from variation in measurement and data fitting, and the masking efficiency of each masked antibody likely falls within the calculated ranges. These results indicate that multiple masking peptides identified from the CPLs maintained their masking efficiency when expressed in mammalian cells, and as part of a full IgG molecule.

TABLE C
Measurement of KDs of masked anti-CD137 antibody
via FACS-based assay prior to protease cleavage

| | KD | | | Masking |
|---|---|---|---|---|
| Antibody | M | nM | R2 | efficiency |

DATA BATCH 1

| | | | | |
|---|---|---|---|---|
| TY21242 | 2.82E−10 | 0.282 | 0.9894 | 1 |
| (parental) | | | | |
| TY22591 | 1.08E−08 | 10.8 | 0.9987 | 38 |
| TY22594 | 2.36E−08 | 23.6 | 0.9916 | 84 |
| TY22595 | 1.07E−07 | 106.8 | 0.9989 | 379 |
| TY22596 | 5.86E−09 | 5.86 | 0.9882 | 21 |
| TY22598 | 8.61E−09 | 8.61 | 0.9843 | 31 |
| TY22599 | 9.81E−08 | 98.1 | 0.9972 | 348 |

DATA BATCH 2

| | | | | |
|---|---|---|---|---|
| TY21242 | 2.92E−10 | 0.292 | 0.9793 | 1 |
| (parental) | | | | |
| TY22595 | 4.12E−08 | 41.2 | 0.9809 | 141 |
| TY22599 | 7.17E−08 | 71.7 | 0.9956 | 246 |
| TY23565 | 3.50E−08 | 35.0 | 0.9915 | 120 |
| TY23566 | 6.43E−08 | 64.2 | 0.9803 | 220 |
| TY23567 | 2.62E−07 | 261.9 | 0.9875 | 898 |
| TY23568 | 1.07E−08 | 10.7 | 0.9936 | 37 |
| TY23569 | 6.16E−08 | 61.6 | 0.9906 | 211 |
| TY23570 | 7.08E−08 | 70.8 | 0.9925 | 243 |
| TY23571 | 2.37E−08 | 23.7 | 0.9896 | 81 |
| TY23572 | 8.49E−08 | 84.9 | 0.9941 | 291 |
| TY23573 | 2.39E−07 | 239.1 | 0.9959 | 820 |

DATA BATCH 3

| | | | | |
|---|---|---|---|---|
| TY21242 | 3.55E−10 | 0.355 | 0.9901 | 1 |
| TY22586 | 1.28E−07 | 128.4 | 0.9929 | 361 |
| TY22599 | 4.40E−08 | 44.0 | 0.9898 | 124 |
| TY23573 | 9.93E−08 | 99.3 | 0.9873 | 279 |
| TY25153 | 2.14E−08 | 21.4 | 0.9557 | 60 |
| TY25154 | 2.22E−08 | 22.2 | 0.9841 | 62 |
| TY25155 | 4.02E−07 | 402.0 | 0.9979 | 1131 |

DATA BATCH 4

| | | | | |
|---|---|---|---|---|
| TY21242 | 1.99E−10 | 0.1994 | 0.9785 | 1 |
| TY23310 | 1.76E−10 | 0.1762 | 0.9066 | 1 |
| TY25155 | 1.83E−07 | 182.5 | 0.9867 | 915 |
| TY25366 | 6.46E−08 | 64.56 | 0.9679 | 324 |
| TY25368 | 1.33E−07 | 133.1 | 0.9885 | 755 |
| TY25369 | 1.78E−07 | 177.8 | 0.9907 | 1009 |
| TY25370 | 4.22E−08 | 42.22 | 0.9898 | 240 |
| TY25371 | 6.19E−08 | 61.91 | 0.9687 | 351 |
| TY25372 | 7.09E−08 | 70.89 | 0.9898 | 402 |

### Example 2: Activities of Masked Anti-CD137 Antibodies Prior to and Following Removal of the Masking Peptide

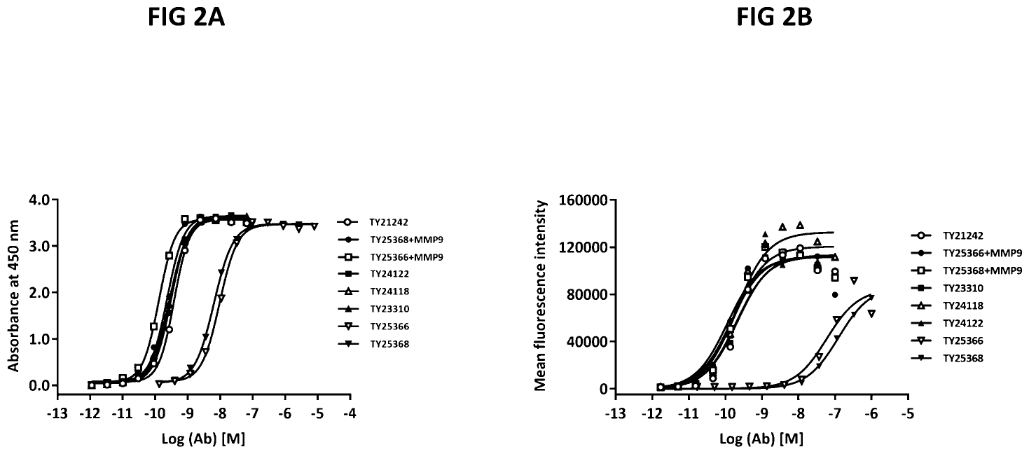

The purified masked antibodies were treated with the proteases that recognize the cleavage sequences in the linkage units. Following treatment, the masked antibodies were then tested to determine whether removal of the masking peptide restores their activity. As an example, 20 μg of TY25366 and TY25368 (0.5 mg/mL) were each treated with 1 μg of recombinant human MMP-9 (in-house) in reaction buffer (50 mM Tris, 10 mM CaCl₂, 150 mM NaCl, 0.05% Brij35(w/v), pH 7.5). The reactions were carried out at 37° C. for 24 hours. The masking peptides were confirmed to be removed from the light chain by ELISA and FACS based assays. As shown in FIGS. 2A-2B, and Table D, after removal of masking peptide, the binding of the masked anti-CD137 antibodies to hCD137 was indistinguishable from the parent antibodies (i.e., TY21242 and TY23310).

TABLE D
ELISA EC50 and FACS KD after protease cleavage of masked anti-CD137 antibodies

| Antibody | | ELISA EC50 | | FACS KD | |
|---|---|---|---|---|---|
| and | | | Masking | | Masking |
| Condition | Description | nM | efficiency | nM | efficiency |
| TY24118 | Parental antibody of masked antibody TY25368. | 0.275 | 1 | 0.115 | 1 |
| | Does not have masking peptide. | | | | |
| | IgG1 variant comprising S267E, L328F mutations (EU | | | | |
| | numbering). | | | | |
| TY25368 + | Masked antibody derived from TY24118. | 0.235 | 0.8 | 0.171 | 1.5 |
| MMP9 | Has masking peptide (SEQ ID NO: 34). | | | | |
| | IgG1 variant comprising S267E, L328F mutations (EU | | | | |
| | numbering). | | | | |
| | Treated with MMP9. | | | | |
| TY24122 | Parental antibody of masked antibody TY25366. | 0.310 | 1 | 0.141 | 1 |
| | Does not have masking peptide. | | | | |
| | IgG4 variant comprising S267E, L328F mutations (EU | | | | |
| | numbering). | | | | |
| TY25366 + | Masked antibody derived from TY24122 | 0.132 | 0.4 | 0.197 | 1.4 |
| MMP9 | Has masking peptide (SEQ ID NO: 34). | | | | |
| | IgG4 variant comprising S267E, L328F mutations (EU | | | | |
| | numbering). | | | | |
| | Treated with MMP9. | | | | |

### Example 3: Developability Profiles of Masked Anti-CD137 Antibodies

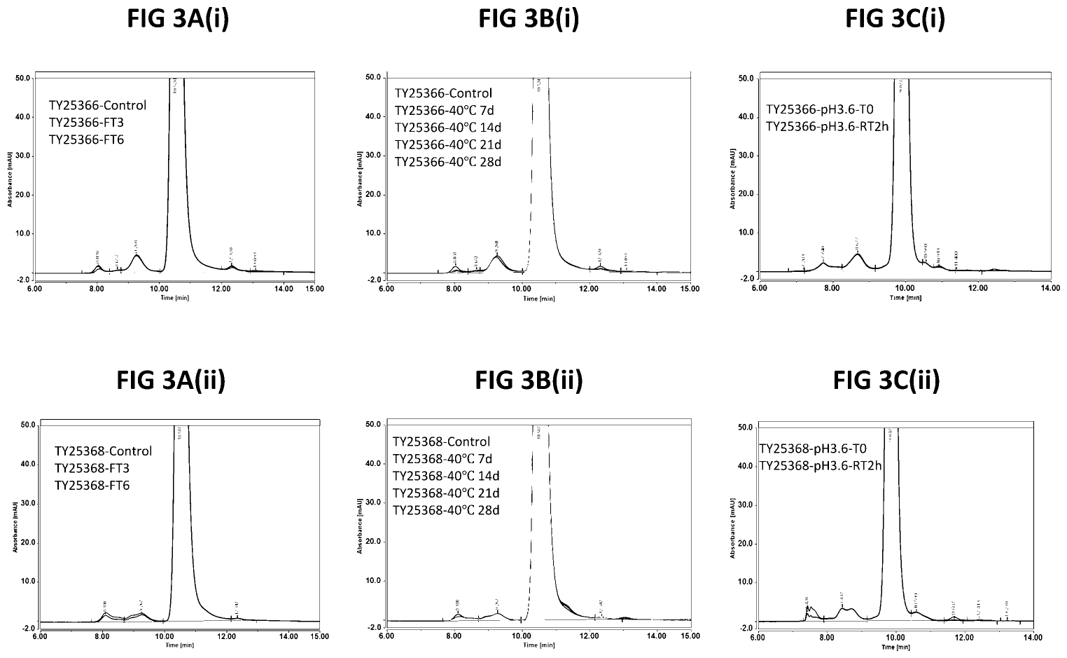

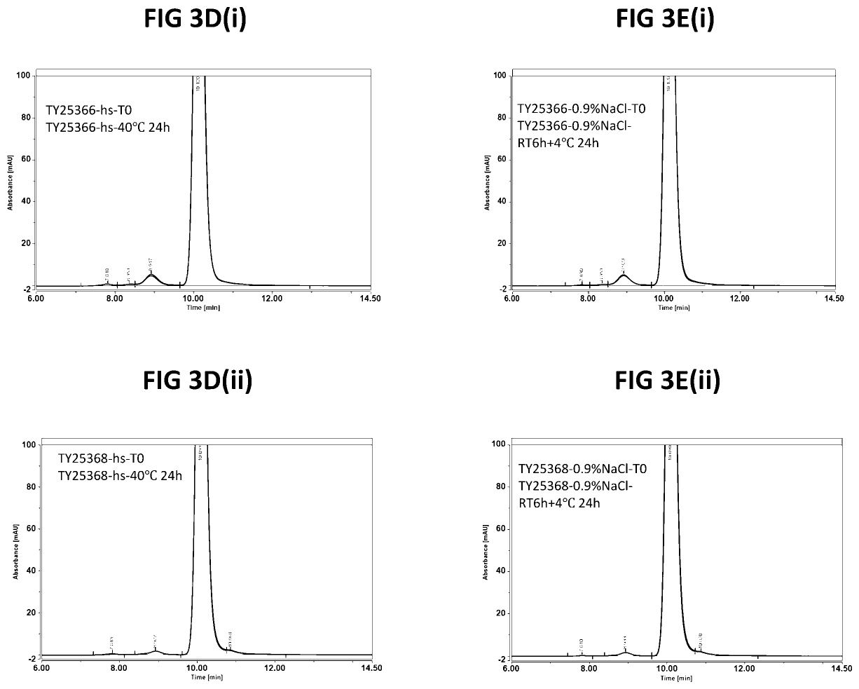

For manufacturing purpose, it is critical that the masked anti-CD137 antibodies have good developability profiles. As described in detail in this example, different assays were performed with purified masked antibodies that were expressed in mammalian cells. The masked antibodies were adjusted to 1 mg/mL in 20 mM Histidine, pH 5.5, and antibody quality analysis was performed using analytical size-exclusion chromatography (SEC) using a Thermo U3000 with a Thermo DAD detector and a XBridge BEH SEC column (7.8 mm×300 mm) (Waters). For each assay, 40 μg of antibody was injected, and fractionation was performed at a flow rate of 0.7 mL/min in buffer (50 mM phosphate within 300 mM sodium chloride at pH 6.8). Five accelerated stress tests were conducted as shown in FIGS. 3A-3E. Briefly, TY25366 and TY25368 were subject to (1) 0, 3, and 6 cycles of freezing and thawing; (2) 0, 7, 14, 21, and 28 days of storage at 40′C; (3) storage in acidic buffer (sodium acetate solution, pH3.6) for 0 and 2 hours at room temperature; (4) storage in 50 mM Histidine, 300 mM NaCl, pH7.0 buffer for 0 and 24 h at 40′C; and (5) storage in saline for 6 h at room temperature followed by storage at 24 h at 4° C. The SEC profiles of TY25366 and TY25368 following storage at accelerated storage condition were similar to the SEC profiles of TY25366 AND TY25368 at T=0 (i.e., prior to storage under the aforementioned conditions). The masked antibodies have not yet gone through an extensive buffer optimization process, and the stability of the masked antibodies may be further improved with optimized buffer and excipient. Taken together, these data indicate that the masked anti-CD137 antibodies remained stable under various stress conditions, and thus have good developability profile.

### Example 4: Binding of Anti-CD137 Antibodies TY24118 and TY24122 to FcγR

The binding of anti-CD137 antibodies TY24118 and TY24122 (i.e., the parental antibodies of masked antibodies TY25368 and TY25366, respectively) to Fcγ receptors was assessed as follows. 2 mg/ml of His-tagged recombinant human FcγR proteins were captured by anti-Penta-His sensor in Fortebio. Then a serially diluted tested antibody (i.e., TY24118 or TY24122) was flowed through the sensor for association, followed with a dissociation step in running buffer. Affinities of the antibodies for the FcγR proteins were calculated by steady state analysis. As shown in Table E, TY24118 and TY24122 exhibited enhanced binding affinity for human FcγRIIa and FcγRIIb proteins, as compared to their corresponding wild type counterpart antibodies, i.e., TY23310 and TY21242, respectively.

TABLE E
Binding affinity KD (nM) of TY24118 and TY24122 to human FcγR

| Antibody | Description | hFcγRIIa | hFcγRIIb |
|---|---|---|---|
| TY23310 | Parent of antibody TY24118. | 970 | 2300 |
| | Does not have masking peptide. | | |
| | WT IgG1 Fc region. | | |
| TY24118 | Parent of masked antibody TY25368. | 6 | 15 |
| | Does not have masking peptide. | | |
| | IgG1 Fc variant comprising S267E and L328F substitutions (EU | | |
| | numbering). | | |
| TY21242 | Parent of antibody TY24122. | 1800 | 2300 |
| | Does not have masking peptide. | | |
| | WT IgG4 Fc region. | | |
| TY24122 | Parent of masked antibody TY25366. | 5.85 | 5.06 |
| | Does not have masking peptide. | | |
| | IgG4 Fc variant comprising S267E and L328F substitutions (EU | | |
| | numbering). | | |

### Example 5: Binding of a Masked Anti-CD137 Antibody to Target T Cells

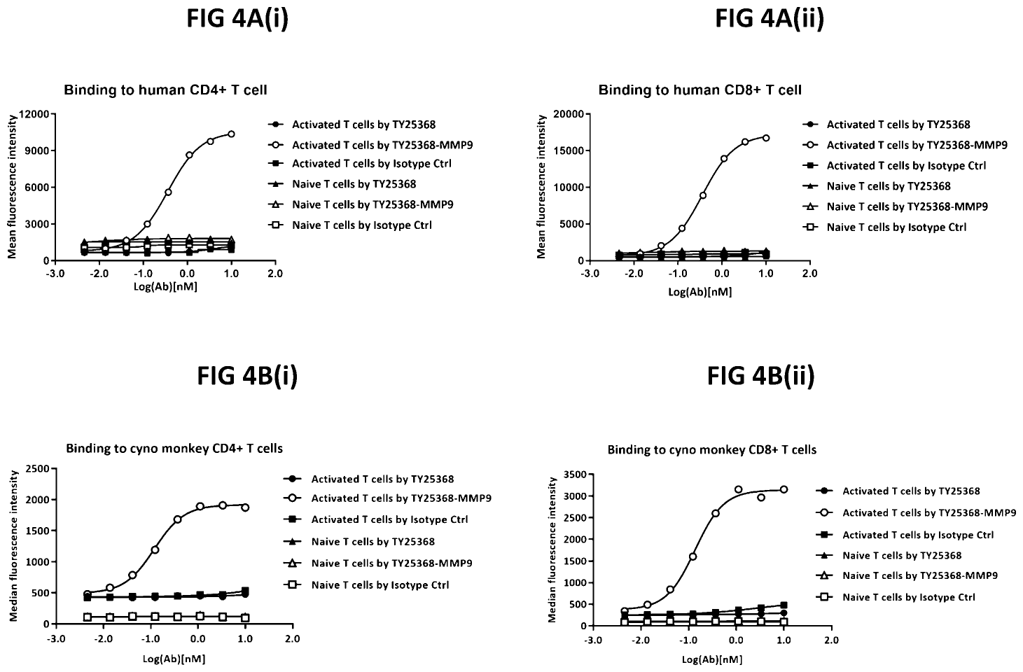

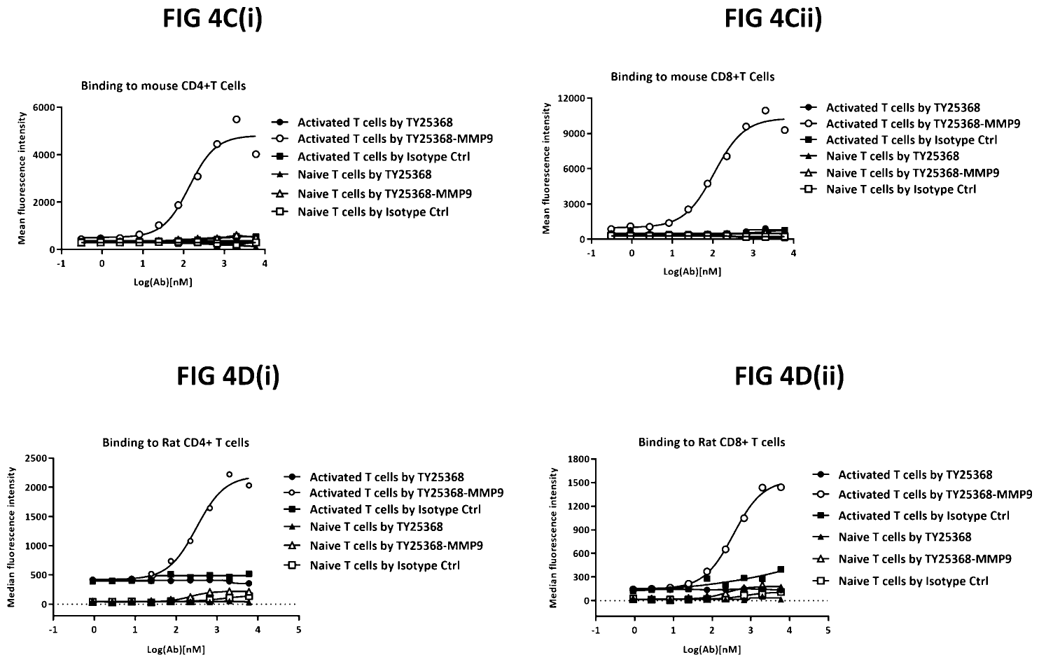

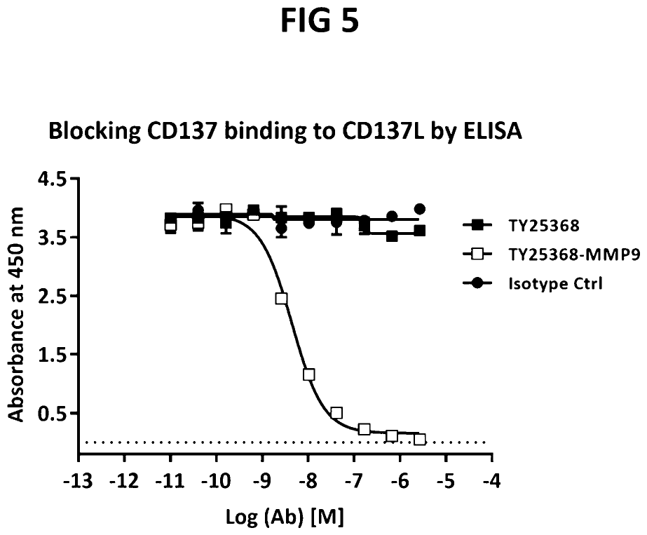

The binding of masked anti-CD137 antibodies to target T cells was assessed as follows. Human and cynomolgus monkey T cells were isolated from peripheral blood obtained from healthy donors. Mouse and rat T cells were isolated from spleen. All T cells were cultured in the presence or absence of anti-CD3/anti-CD28 stimulation. Activated T cells or unstimulated naïve T cells were incubated with serially diluted test antibodies together with corresponding anti-CD4 and anti-CD8 antibodies to gate T cell subpopulations. The binding of test antibodies was detected with a fluorescently labeled anti-human IgG Fc secondary antibody by FACS analysis. As shown in FIGS. 4A-D, only MMP9-cleaved TY25368 showed positive staining on activated human T cells, cynomolgus T cells, mouse T cells, and rat T-cells, consistent with the induced CD137 expression in activated T cells. MMP9-cleaved TY25368 showed no detectable binding to unstimulated naïve T cells. Uncleaved TY25368 showed no detectable binding to activated or naïve T cells. These results suggest that the masking peptide of masked antibody TY25368 prevented its binding to the target T cells and that unmasking by protease cleavage restored TY25368's target cell binding activity.

### Example 6: Ligand Blocking Activity of a Masked Anti-CD137 Antibody

The ligand blocking activity of anti-CD137 masked antibodies was assessed as follows: 1 mg/ml of recombinant human CD137 protein was coated onto ELISA plates. Next, 2 mg/ml biotinylated recombinant human CD137 ligand was incubated with the CDI 37 pre-coated ELISA plates in the presence of serially diluted masked antibodies for 1 hour at 37° C. After washing, NeutrAvidin-HRP was added to the plates to detect the interaction between CD137 and its ligand. As shown in FIG. 5, MMP9-cleaved TY25368 showed strong inhibition of the interaction between CD137 and its ligand in a dose-dependent manner, with IC50 at 4.46 nM. Uncleaved TY25368 showed no detectable inhibitory activity, similar as the isotype control antibody. These results indicate that upon cleavage, TY25368 was able to block ligand interaction with CD I37 receptor.

### Example 7: Activation of CD137-Mediated Cell Signaling by a Masked Anti-CD137 Antibody

The stimulatory activities on CDI 37 receptor signaling by the anti-CD137 antibodies and their masked counterparts were evaluated using Jurkat-CD137-NFkB-luciferase reporter cells. Briefly, the reporter cells were cultured in the presence or absence of the CHO-K1-hFcγRIIb cells as cross-linker (E:CL=20:1, where “E” refers to Jurkat-CD137-NFkB-luciferase reporter cells and “CL” refers to CHO-K1-hFcγRIIb cells). Serially diluted test antibodies were added into the reporter cell system to evaluate their activity in stimulating downstream luciferase activity.

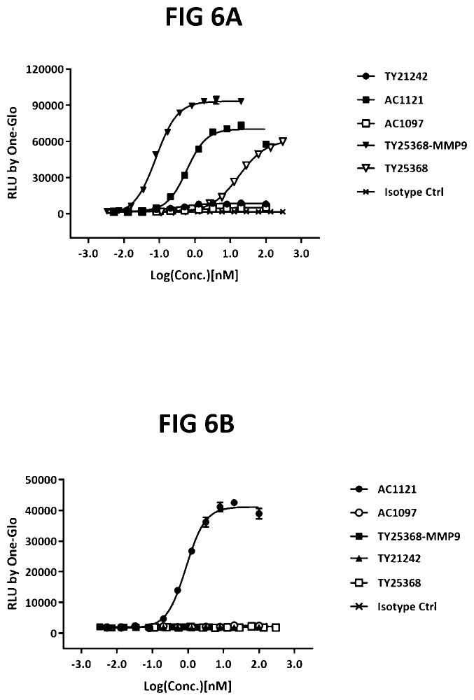

As shown in FIGS. 6A-6B and Table F, in the presence of CHO-K1-hFcγRIIb cells as cross-linker, all anti-CD137 antibodies showed activation of CD137 signaling. MMP9-treated masked antibody TY25368 exhibited the strongest signal. The activity of the uncleaved masked antibody TY25368 was much weaker than its cleaved form. Other clinical anti-CD137 antibodies were also compared in the assay. Anti-CD137 antibodies AC1121 and AC1097 are described in WO 2019/036855 and US 2020/00369776, the contents of which are incorporated by reference herein in their entireties. Notably, in the absence of CHO-K1-hFcγRIIb cells as cross-linker, only AC1 121 showed activity. All other anti-CD137 antibodies were inactive, suggesting their CD137 agonistic activity is crosslinking dependent.

TABLE F
Stimulation of CD137-mediated signaling by Anti-CD137 antibodies
in the presence or absence of CHO-K1-hFcγRIIb cross-linker cells

| | | EC50 (nM) in the | EC50 (nM) in the |
|---|---|---|---|
| | | presence of CHO- | absence of CHO- |
| | | K1-hFcγRIIb cells | K1-hFcγRIIb cells |
| Antibody | Description | as cross-linker | (no cross-linker) |
| TY25368 + | Masked antibody of TY24118, which is derived from | 0.08 | NA |
| MMP9 | TY21242. | | |
| TY25368 (no | Has masking peptide (SEQ ID NO: 34). | 17.29 | NA |
| MMP9) | IgG1 Fc variant comprising S267E and L328F | | |
| | substitutions (EU numbering). | | |
| TY23310 | Parent of TY24118, which is parent of TY25368. | 0.27 | NA |
| | Does not have peptide with masking sequence and | | |
| | linkage unit. | | |
| | WT IgG1 Fc region. | | |
| AC1121 | Described in WO 2019/036855 and US 2020/00369776. | 0.59 | 0.88 |
| | Does not have peptide with masking sequence and | | |
| | linkage unit. | | |
| AC1097 | Described in WO 2019/036855 and US 2020/00369776. | 1.08 | NA |
| | Does not have peptide with masking sequence and | | |
| | linkage unit. | | |

### Example 8: Human Primary B Cell Cross-Linking Dependent Activation by a Masked Anti-CD137 Antibody

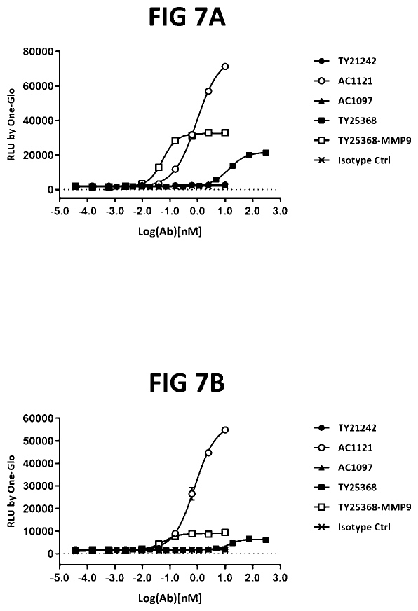

The stimulatory activities on CD137 receptor signaling by anti-CD137 antibodies and their masked counterparts were evaluated as follows. Human primary B cells were isolated from peripheral blood obtained from healthy donors. Jurkat-CD137-NFkB-luciferase reporter cells were cultured in the presence or absence of primary B cells as cross-linker (E:CL=5:1 or 20:1). Serially diluted test antibodies were added to the reporter cell system to evaluate their abilities to stimulate downstream luciferase activity. As shown in FIG. 7, in the presence of human primary B cells as cross-linker, in both E:CL ratios (5:1 or 20:1) the cleaved TY25368-MMP9 exhibited the strongest CD137 activation signal. The activity of the uncleaved masked antibody TY25368 was much weaker than its cleaved form. Other clinical anti-CD137 antibodies were also compared in the assay.

### Example 9: Staphylococcal Enterotoxin a (SEA)-Induced Activation of Peripheral Blood Mononuclear Cells by a Masked Anti-CD137 Antibody

Anti-CD137 antibodies were evaluated as follows to determine whether they were capable of enhancing Staphylococcal enterotoxin A (SEA) peptide stimulated human T-cell activation in the context of human peripheral blood mononuclear cells (PBMCs). SEA, a super-antigen, is known to activate a large fraction of human T cells by binding to MHC II expressed on the surfaces of antigen presenting cells and T cell receptors (TCRs) expressed on the surfaces of T cells and was thus chosen to prime T cell activation in this study. Human PBMCs (2.0×10⁵/well of a 96-well plate) were isolated from two healthy donors (Donor #102 and Donor #142) and stimulated with a sub-optimal concentration of the SEA peptide (50 ng/mL). Next, serially diluted concentrations of anti-CD137 antibodies, as well as an isotype control antibody, were aliquoted into the wells. Replicate cell supernatants were collected after 4 days for measurement of IL-2 with ELISA as an endpoint for enhanced T cell activation.

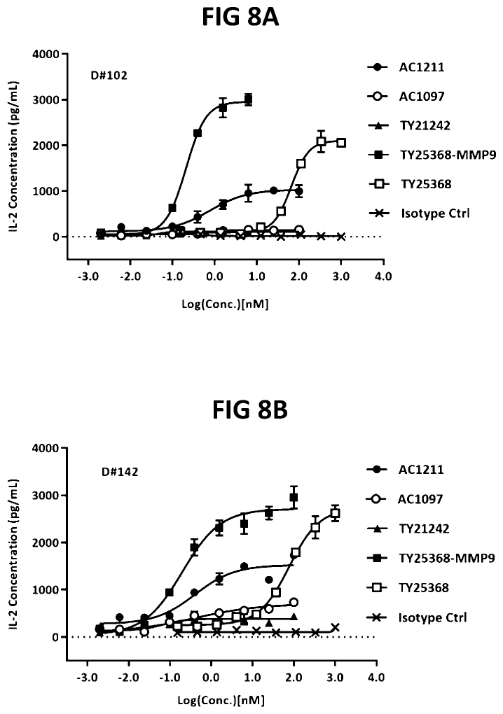

As shown in FIG. 8 and Table G, PBMC from both donors exhibited the highest level of IL-2 cytokine secretion in the presence of cleaved TY25368 (i.e., TY25368 treated with MMP9). Levels of IL-2 cytokine secretion by PBMC from both donors was much weaker in the presence of uncleaved TY25368. Other clinical anti-CD137 antibodies (i.e., AC1121 and AC1097) were also tested in this assay.

TABLE G
Enhancement of SEA-Induced Activation of PBMC by Anti-CD137 Antibodies

| | | EC50 (nM) | EC50 (nM) |
|---|---|---|---|
| | | PBMC from | PBMC from |
| Antibody | Description | Donor #102 | Donor #102 |
| AC1121 | Described in WO 2019/036855 and US 2020/00369776. | 0.767 | 0.404 |
| | Does not have peptide with masking sequence and | | |
| | linkage unit. | | |
| AC1097 | Described in WO 2019/036855 and US 2020/00369776. | 0.926 | 0.385 |
| | Does not have peptide with masking sequence and | | |
| | linkage unit. | | |
| TY23310 | Parent of TY24118, which is parent of TY25368. | 0.047 | 0.068 |
| | Does not have masking peptide. | | |
| | WT IgG1 Fc region. | | |
| TY25368 + | Masked antibody of TY24118, which is derived from | 0.209 | 0.199 |
| MMP9 | TY21242. | | |
| TY25368 | Has masking peptide (SEQ ID NO: 34). | 65.290 | 77.07 |
| (no MMP9) | IgG1 Fc variant comprising S267E and L328F | | |
| | substitutions (EU numbering). | | |

### Example 10: Screening Anti-CD137 Antibodies for Antibody-Dependent Cell-Mediated Cytotoxicity (ADCC) and Complement-Dependent Cytotoxicity (CDC)

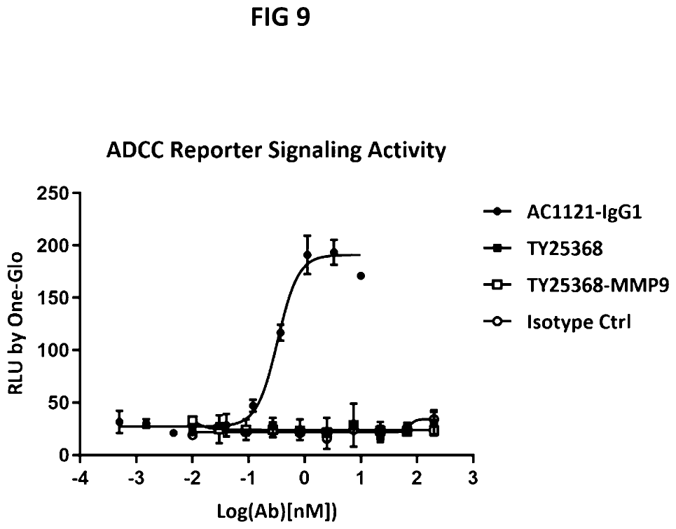

Anti-CD137 antibodies and masked anti-CD137 antibodies were screened for antibody-dependent cell-mediated cytotoxicity (ADCC) activity as follows. Jurkat-CD16-NFAT-luciferase cells were co-cultured with 293-CD137 cells (i.e., 293F cell engineered to overexpress hCD137) as target cells. Serially diluted test antibodies were added into the ADCC reporter cell system to evaluate their abilities to stimulate the downstream luciferase activity. As shown in FIG. 9, ADCC reporter activity was not detected on 293F-CD137 cells in the presence of MMP9-treated TY25368, untreated TY25369, or an IgG1 isotype control antibody. By contrast, AC1121-IgG1 showed strong ADCC reporter activity on 293F-CD137 cells in a dose-dependent manner.

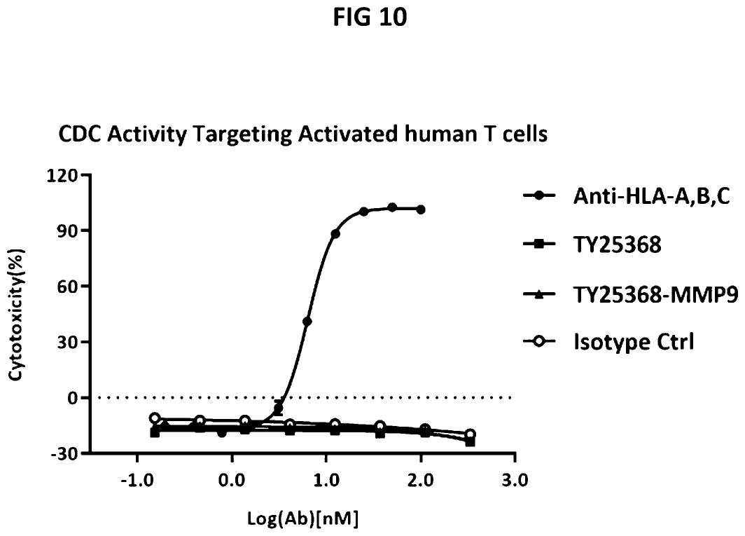

Masked anti-CD137 antibody TY25368 was screened for complement-dependent cytotoxicity (ADCC) as follows. Human T cells isolated from a healthy donor were activated with anti-CD3/anti-CD28 to induce high levels of CD137 and HLA-A/B/C expression. Activated T cells were cultured in the presence of normal human serum complement (NHSC). Next, serially diluted test antibodies were added into the assay system. As shown in FIG. 10, CDC activity was not detected in the presence of MMP9-treated TY25368, untreated TY25368, or an IgG1 isotype control antibody. By contrast, a positive control antibody (i.e., mouse anti-human HLA-A/B/C) showed strong CDC activity on activated T cells in a dose-dependent manner.

### Example 11: Anti-Tumor Efficacy in a CT26 Murine Colon Tumor Model

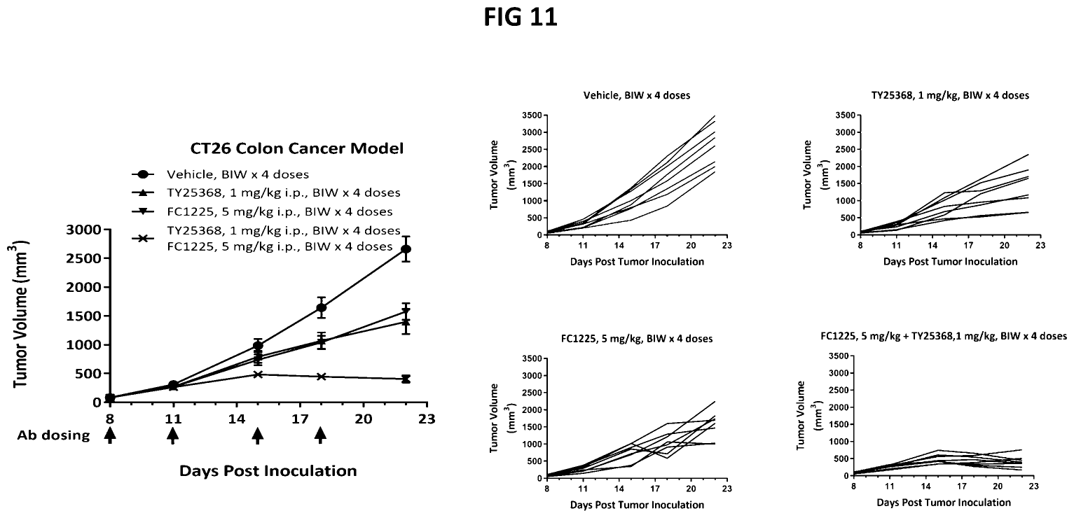

BALB/c mice (female, 8-9 weeks old) were inoculated subcutaneously with CT26 murine colon cancer cells. When tumors reached a volume of ˜80 mm³, the mice were randomly divided into four groups (n=8 per group) and treated with (a) vehicle control, (b) 1 mg/kg masked antibody TY25368, (c) 5 mg/kg anti-PD-1 antibody FG1225, or (d) 1 mg/kg masked antibody TY25368 and 5 mg/kg anti-PD-1 antibody FG1225. The treatments were administered twice a week via by intraperitoneal injection. Tumor growth was monitored twice a week and reported as the mean tumor volume±standard or error measurement (SEM) over time. As shown in FIG. 11, TY25368 and FG1225 each showed partial efficacy as single agent. On Day 22 following the start of treatment, tumor growth was inhibited by 48% in mice treated with TY25368 and by 42% in mice treated with FC1225. By contrast, tumor growth was inhibited by 85% in mice treated with TY25368 in combination with FC1225.

### Example 12: Anti-Tumor Efficacy in a MC38 Murine Colon Tumor Model

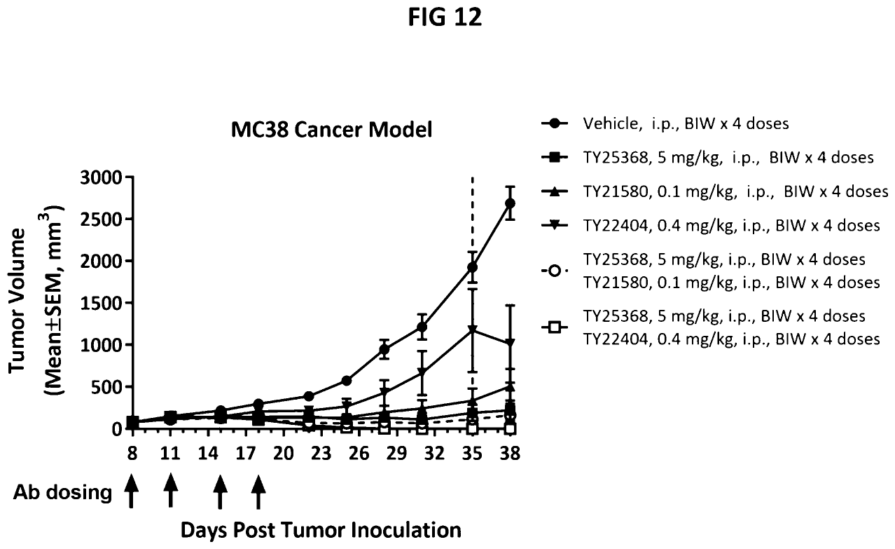

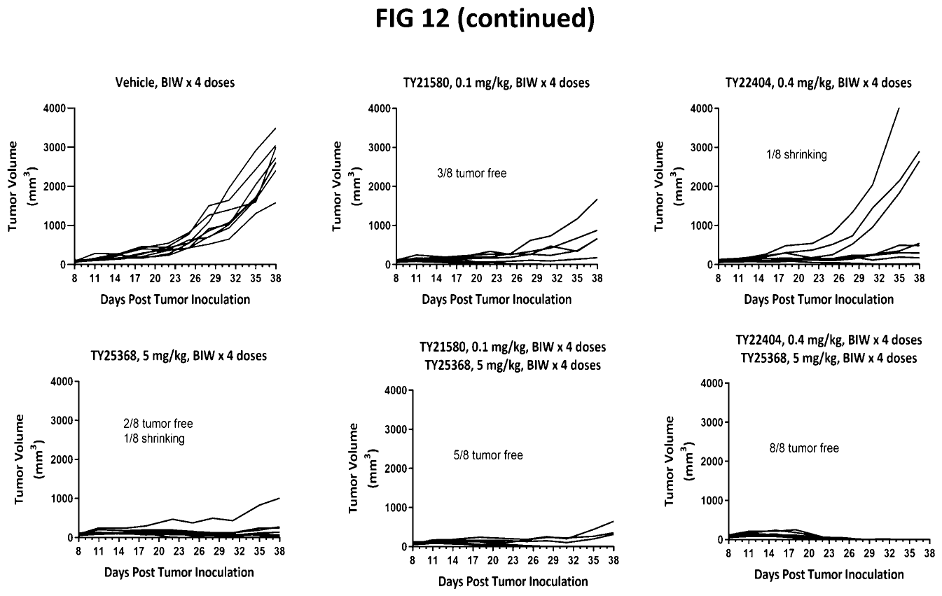

C57BL/6 mice (female, 8-9 weeks old) were inoculated subcutaneously with MC28 murine colon cancer cells. When tumors reached a volume of ˜80 mm³, the mice were randomly divided into eight groups (n=8 per group) and treated with (a) vehicle, (b) 5 mg/kg masked antibody TY25368, (c) 0.1 mg/kg anti-CTLA4 antibody TY21580, (d) 0.4 mg/kg masked anti-CTLA4 antibody TY22404, (e) 5 mg/kg TY25368 and 0.1 mg/kg TY21580, or (f) 5 mg/kg TY25368 and 0.4 mg/kg TY22404. Treatments were administered twice a week via intraperitoneal injection. Tumor growth was monitored twice a week and reported as the mean tumor volume t SEM over time. As shown in FIG. 12, TY21580 and TY22404 each showed partial efficacy as single agents. 3 of the 8 mice treated with single agent TY21580 mono treatment group were tumor free. Tumor growth was inhibited in 1 of the 8 mice treated with single agent TY22404. Combination treatment with TY25368 and TY21580, or with TY25368 and TY22404 resulted in significantly enhanced anti-tumor efficacy. See FIG. 12. 5 of the 8 mice treated with TY25368 and TY21580 were tumor-free. All 8 mice treated with TY25368 and TY22404 were tumor-free.

### Example 13: A Pharmacokinetic (PK) and Pharmacodynamics (PD) Study of TY25368 in Cynomolgus Monkeys

Cynomolgus monkeys given either 30 mg/kg TY25368 or 100 mg/kg TY25368 via intravenous administration once per week for two weeks. The plasma concentrations of antibody TY25368 (both intact (i.e., uncleaved masked antibody) and total (i.e., both uncleaved and cleaved forms) were measured at different time points using ELISA assays. The results are Table H below. Peripheral T lymphocytes were also profiled by FACS analyses, and no significant changes were observed (data not shown). The monkeys tolerated well with the antibody administration, and no clinical signs noted during the study.

TABLE H
PK Parameters of TY25368 in Cynomolgus Monkeys

| | Dose | Study | | Cmax | Tmax | AUC_{0-168.317 h} | T½ | Vd | Cl |
|---|---|---|---|---|---|---|---|---|---|
| | (mg/kg) | Day | Sex | (ng/mL) | (h) | (h*ng/mL) | (h) | (mL/kg) | (mL/h/kg) |
| TY25368 (total) | 30 | 1 | Male | 504000 | 0.5 | 39800000 | 136.3 | 85.2 | 0.447 |
| | | 8 | Male | 639000 | 0.5 | 42800000 | 32.6 | 43.0 | 0.664 |
| TY25368 (intact) | 30 | 1 | Male | 526000 | 0.5 | 41800000 | 273.0 | 101 | 0.265 |
| | | 8 | Male | 773000 | 0.5 | 48200000 | 31.0 | 37.4 | 0.594 |

| | Dose | Study | | Cmax | Tmax | AUC_{0-169 h} | T½ | Vd | Cl |
|---|---|---|---|---|---|---|---|---|---|
| | (mg/kg) | Day | Sex | (ng/mL) | (h) | (h*ng/mL) | (h) | (mL/kg) | (mL/h/kg) |
| TY25368 (total) | 100 | 1 | Male | 1570000 | 2.0 | 130000000 | 190.4 | 95.9 | 0.348 |
| | | 8 | Male | 1890000 | 2.0 | 197000000 | 158.1 | 60.5 | 0.267 |
| TY25368 (intact) | 100 | 1 | Male | 18C40000 | 1.2 | 141000000 | 201.2 | 89.3 | 0.304 |
| | | 8 | Male | 2380000 | 1.2 | 223000000 | 144.7 | 51.7 | 0.244 |

Cmax = maximum (peak) serum concentration; Tmax = time to reach Cmax; AUC = area under curve that describes the variation of a drug concentration in blood plasma as a function of time; T½ = half-life; Vd = total amount of drug in entire body/drug plasma concentration; Cl = clearance.

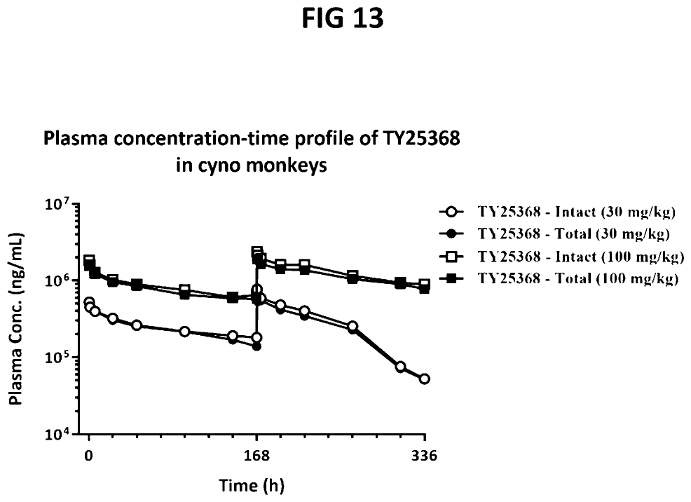

As shown in FIG. 13 and Table H, TY25368 showed linear a PK profile after the first dosing at dose range between 30 mg/kg and 100 mg/kg for both Total and Intact form. Following the second 30 mg/kg dose, TY25368 exhibited fast clearance and short T1/2. Without being bound by theory, such effect may be due to treatment induced anti-drug antibody (ADA). Comparing the Total form and Intact form after dosing, highly similar drug concentrations and drug exposures were observed, indicating that masked antibody TY25368 was stable in the peripheral blood in monkeys. Thus, pre-clinical toxicology studies demonstrate that TY25366 and TY25368 are well-tolerated in monkeys with normal pharmacokinetic behaviors and minimal activation in circulation.

### Example 14: A Pharmacokinetic (PK) Study of TY21242, TY24118, and TY25366 in Mice

A pharmacokinetic study was conducted in BALB/c female mice bearing CT26 tumor. The mice were randomly divided into four groups (n=4 per group) and were intraperitoneally injected with 5 mg/kg of TY25368, TY25366, TY21242, TY24118, or TY24122. Blood samples (˜50 μl per sample) were collected at 3, 6, 24, 48, 96, 168 and 336 hours post-dosing. The blood concentrations of TY25368, TY25366, TY21242, TY24118, and TY24122 were determined by ELISA using an anti-human IgG Fc antibody as the capture agent and an HRP-labeled anti-human IgG (Fab specific) antibody as the detection agent. A second set of ELISA assays, using a specific anti-idiotype antibody as capture agent and an HRP-labeled anti-human IgG (Fab specific) antibody as a detection agent, was performed to detect the active (i.e., unmasked) forms of TY25368 and TY25366. Descriptions of each antibody are provided in Table I.

TABLE I
Antibody Descriptions

| Antibody | Description |
|---|---|
| TY23310 | Parent of TY24118, which is parent of masked antibody TY25368. |
| | Does not have masking peptide. |
| | WT IgG1 Fc region. |
| TY24118 | Parent of masked antibody TY25368. |
| | Does not have masking peptide. |
| | IgG1 Fc variant comprising S267E and L328F substitutions (EU numbering). |
| TY25368 | Masked anti-CD137 antibody derived from TY24118. |
| | Has masking peptide (SEQ ID NO: 34). |
| | IgG1 Fc variant comprising S267E and L328F substitutions (EU numbering). |
| TY21242 | Parent of TY24122, which is parent of masked antibody TY25366. |
| | Does not have masking peptide. |
| | WT IgG4 Fc region. |
| TY24122 | Parent of masked antibody TY25366. |
| | Does not have masking peptide. |
| | IgG4 Fc variant comprising S267E and L328F substitutions (EU numbering). |
| TY25366 | Masked anti-CD137 antibody derived from TY24122. |
| | Has masking peptide (SEQ ID NO: 34). |
| | IgG4 Fc variant comprising S267E and L328F substitutions (EU numbering). |

TY25368 had a half-life of 98 hours, and the drug concentration at 336 hours was about 6.48 μg/ml. TY25366 had a half-life of 28 hours, and the drug concentration at 336 hours was about 0.13 μg/ml. In comparison, the parental antibody TY21242 had a half-life of 74 hours, and the drug concentration at 336 hours was about 3.09 μg/ml; TY24118 had a half-life of 77 hours, and the drug concentration at 336 hours was about 2.50 μg/ml; and TY24122 has a half-life of 79 hours, and the drug concentration at 336 hours was about 3.74 μg/ml. TY25368 had a slower clearance time and longer half-life than the parental antibodies, while TY25366 had a much faster clearance time and shorter half-life than the parental antibodies. No active forms of TY25366 or TY25368 were detected (data not shown), which indicates that the masked antibodies are stable in mouse peripheral blood.

Thus, pre-clinical toxicology studies demonstrate that TY25366 and TY25368 are well-tolerated in mice with normal pharmacokinetic behaviors and minimal activation in circulation.

### Example 15: Activation of CD137-Mediated Cell Signaling by Anti-CD137 Antibodies Comprising Fc Mutation(s)

The Fc mutations in Table J, which could effect cross-linking, were introduced into TY23310 or TY21242, to generate TY24117, TY24118, TY24119, TY24120, TY24121 and TY24122.

TABLE J
Anti-CD137 Antibodies Comprising Fc Mutation(s)

| IgG | Mutation | Isotype | Parental IgG |
|---|---|---|---|
| TY24117 | ^{a}S267E | hIgG1 | TY23310 |
| TY24118 | ^{a}S267E/L328F | hIgG1 | TY23310 |
| TY24119 | ^{b}G237D/P238D/P271G/ | hlgG1 | TY23310 |
| | A330R | | |
| TY24120 | ^{b}G237D/P238D/H268D/ | hIgG1 | TY23310 |
| | P271G/A330R | | |
| TY24121 | ^{a}S267E | hIgG4 | TY21242 |
| TY24122 | ^{a}S267E/L328F | hIgG4 | TY21242 |

^{a}see Chu et al. (2008) Mol Immunol. 45(15): 3926-33.
^{b}see Mimoto et al. (2013) Protein Eng Des Sel. 26(10): 589-98

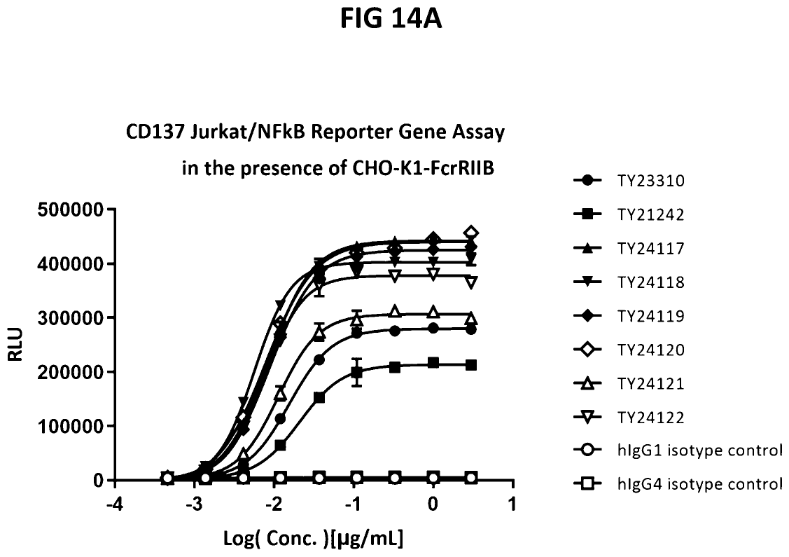

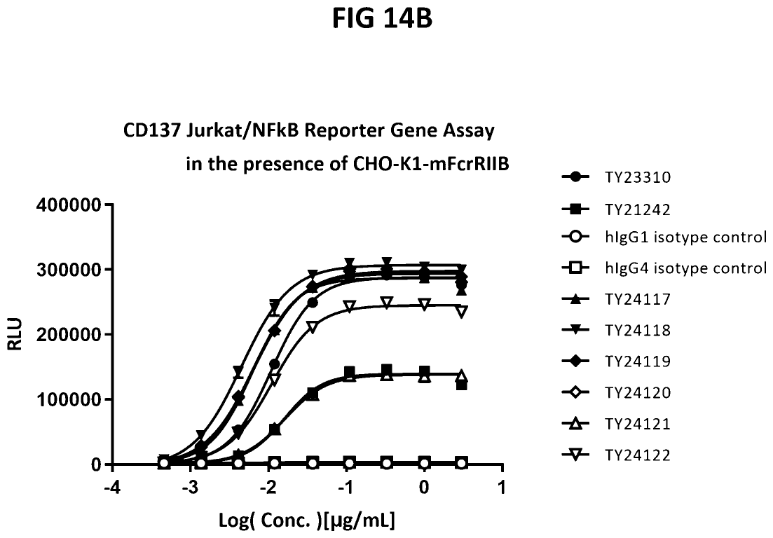

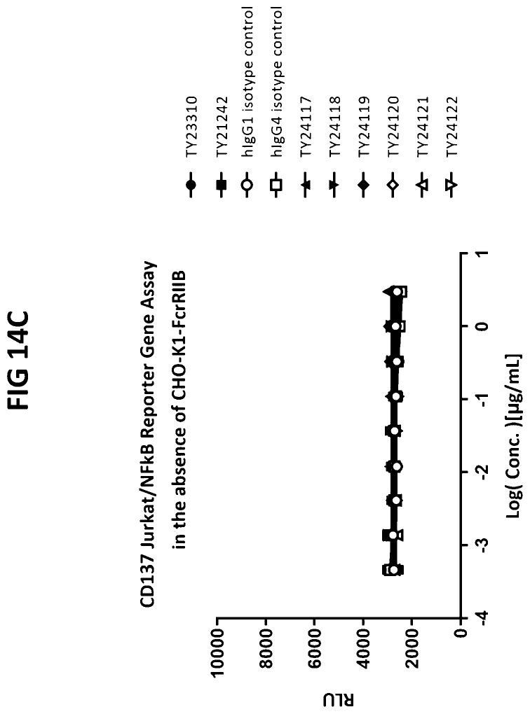

The anti-CD137 antibodies were transiently expressed in HEK293F cells and purified with standard protein A affinity chromatography (MabSelect SuRe, GE Healthcare). The agonistic activity of the anti-CD137 antibodies were compared in a Jurkat/NFkB reporter gene assay. Briefly, human embryonic kidney 293T cells were transiently transfected with plasmids expressing human CD137 receptor, along with NFκB firefly luciferase reporter and control Renilla luciferase reporter constructs. Cells were incubated with the test antibodies in the presence or absence of (a) human CHO-K I-hFcγRIIb (also referred to as CHO-K1-FcγRIIb) or (b) or mouse CHO-K1-mFcγRIIb. The relative levels of CD137 signaling activation were then measured by the Firefly luciferase activity after normalization with the Renilla luciferase activity. As shown in FIGS. 14A-14C, in the presence of CHO-K1-FcγRIIb, both tested antibodies show higher CD137 agonistic activities than TY23310 with wild type IgG1 or TY21242 with wild type IgG4 counterparts. As for CHO-K1-mFcγRIIb as cross-linker, activity enhancement was either not observed or observed to be less than the activity enhancement seen in the human system. In the absence of cross-linker, none of the antibodies exhibited CD137-mediated cell signaling.

### Example 16: Staphylococcal Enterotoxin A (SEA)-Induced Activation of Peripheral Blood Mononuclear Cells by Anti-CD137 Antibodies

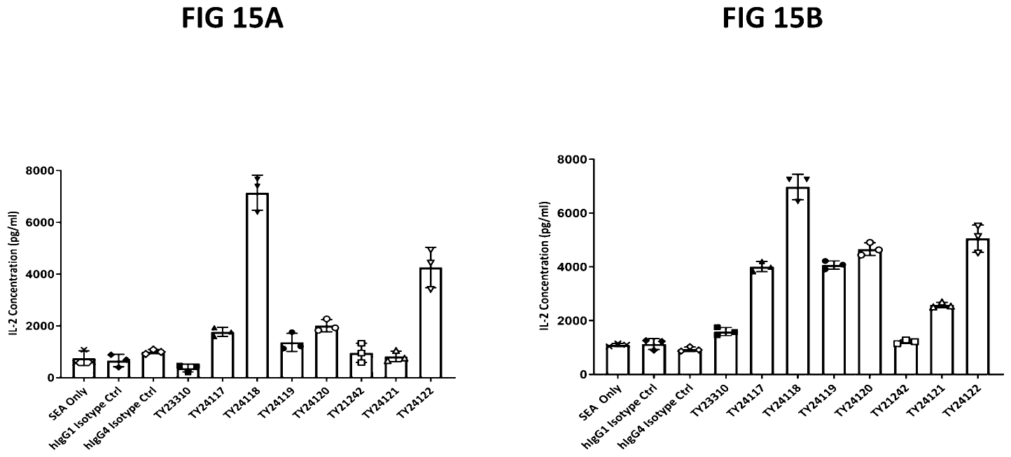

The anti-CD137 antibodies generated in Example 15 were evaluated as follows to determine whether they were capable of enhancing Staphylococcal enterotoxin A (SEA) peptide stimulated human T-cell activation in the context of human peripheral blood mononuclear cells (PBMCs). Fresh human PBMCs were incubated with 50 ng/mL SEA and serially diluted concentrations of anti-CD137 antibodies (in either soluble form or immobilized on a solid support) for 96h, then the cell culture supernatants were collected to measure IL-2 levels. As shown in FIG. 15, anti-CD137 antibodies TY24118 and TY24122 exhibited better agonistic activity than TY24117, TY24118, TY24119, TY24120, TY24121 and TY24122.

### Example 17: Affinities of TY25368 and TY25368-MMP9 for Human CD137, Cynomolgus CD137, Mouse CD137, and Rat CD137

The binding affinities of TY25368 and its activated form, which is referred to as TY25368-MMP9, to recombinant CD137 from different species was assessed by Surface Plasmon Resonance (SPR). Briefly anti-Human IgG (Fc) antibody (Cytiva, catalog #BR-1008-39) was immobilized onto CM5 chips by amide coupling following the instruction of amine coupling kit (Cytiva, catalog #BR-1000-50). Final response of immobilization level were about 5000 RU (relative units). TY25368 and TY25368-MMP9 were each diluted with 1×HBS-EP buffer (10 mM HEPES, 150 mM NaCl, 3 mM EDTA·2Na, 0.005% (v/v) Surfactant P20, pH 7.4) to 3 g/mL, then injected to the system for 30 s at a flow rate of 10 TY6 min for immobilization. CD137 antigens were diluted serially with 1×HBS-EP buffer, and flowed through the test antibody-immobilized CM5 chip for 300 s at flow rate of 30 μL/min. After association, 1×HBS-EP buffer was injected to the surface at flow rate of 30 μL/min for another 300 s to test antigen dissociation rate. 3M magnesium chloride solution was used to remove retained antibody/antigen for CM5 sensor chip regeneration. As shown in Table K, TY25368 has low binding affinity for human CD137, cynomolgus CD137, mouse CD137, and rat CD137 (KD>1000 nM). By contrast, activated TY25368-MMP9 was found to bind human CD137 and cynomolgus CD137 with high affinity (with KDs of 3.19 and 4.23 nM, respectively). Activated TY25368-MMP9 was also found to bind mouse CD137 and rat CD137 with lower affinities (with KDs of 27.54 and 42.37 nM, respectively).

TABLE K
Affinities of TY25368 and TY25368-MMP9 for CD137
from different species, as measured by SPR

| Antigen | Test antibody | KD (nM) |
|---|---|---|
| Human CD137 | TY25368 | >1000 |
| | Activated TY25368 | 3.19 |
| | (TY25368-MMP9) | |
| Cynomolgus | TY25368 | >1000 |
| monkey | Activated TY25368 | 4.23 |
| CD137 | (TY25368-MMP9) | |
| Mouse CD137 | TY25368 | >1000 |
| | Activated TY25368 | 27.54 |
| | (TY25368-MMP9) | |
| Rat CD137 | TY25368 | >1000 |
| | Activated TY25368 | 42.37 |
| | (TY25368-MMP9) | |

### Example 17: Anti-Tumor Efficacy of TY25368 in a Murine EMT6 Breast Cancer Model

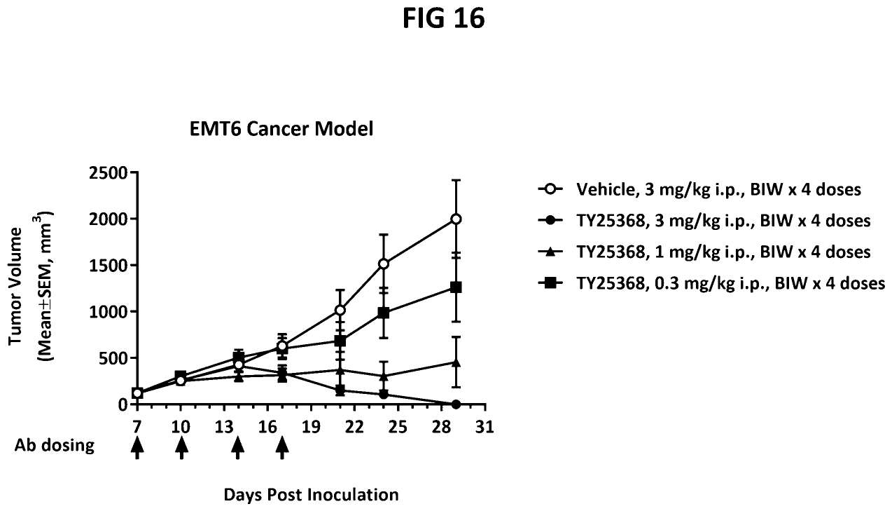

BALB/c mice (4 groups, n=8 per group, female, 8-9 weeks old) were inoculated subcutaneously with EMT6 (ATCC) murine breast cancer cells. When tumors were established (i.e., when tumor volumes reached ˜120 mm³), mice were treated with (a) vehicle, (b) 3 mg/kg masked anti-CD137 antibody TY25368, (c) 1 mg/kg TY25368, or (d) 0.3 mg/kg TY25368. Treatments were administered via intraperitoneal injection twice a week. Tumor growth was monitored twice a week and reported as the mean tumor volume±s.e.m. over time. As shown in FIG. 16, TY25368 showed dose dependent anti-tumor effect in the EMT6 murine allografted tumor model. Tumor regression was observed in all mice treated with 1 mg/kg TY25368 and with 3 mg/kg TY25368.

### Example 18: Anti-Tumor Efficacy TY24118, TY24122, TY25366 and TY25368 in a H22 Murine Liver Cancer Model

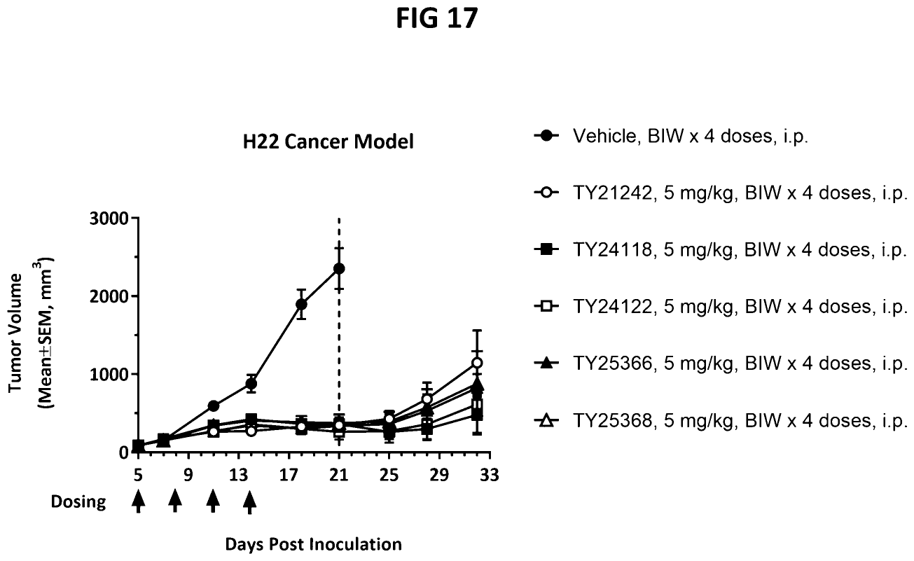

BALB/c mice (5 groups, n=8 per group, female, 8-9 weeks old) were inoculated subcutaneously with H22 (CCTCC) murine liver cancer cells. When tumors were established (i.e., when tumor volumes reached ˜90 mm³), mice were treated with (a) vehicle, (b) 5 mg/kg TY24118, (c) 5 mg/kg TY24122, (d) 5 mg/kg TY25366, or (e) 5 mg/kg TY25368. (Descriptions of the antibodies are provided in Table I above.) Treatments were administered via intraperitoneal injection twice a week. Tumor growth was monitored twice a week and reported as the mean tumor volume±s.e.m. over time. As shown in FIG. 17, the parental anti-CD137 antibodies TY24118 and TY24122, which are not masked, and masked anti-CD137 antibodies TY25366 and TY25368 all showed similar efficacy at 5 mg/kg.

### Example 19: Anti-Tumor Efficacy of TY24118, TY24122, TY25366 and TY25368 in a Murine a CT26 Colorectal Cancer Model

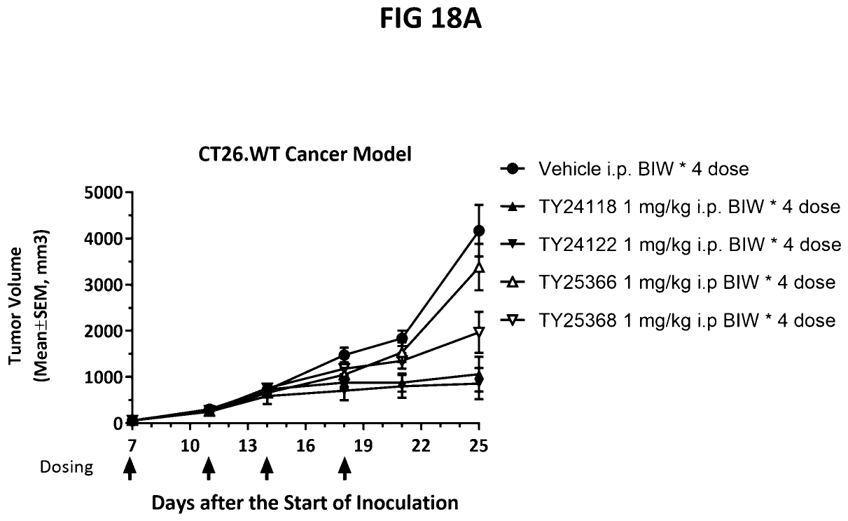

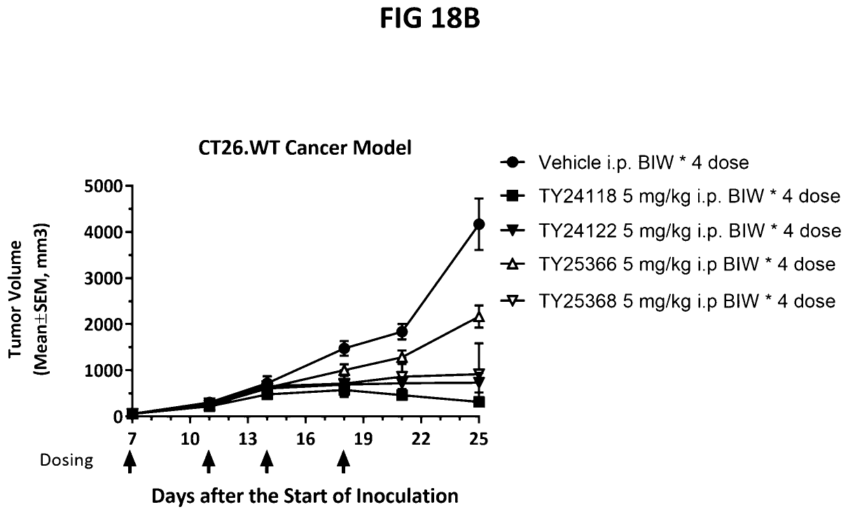

BALB/c mice (5 groups, n=8 per group, female, 9-10 weeks old) were inoculated subcutaneously with CT26 (SIBS) murine colon cancer cells. When tumors were established (i.e., when tumor volumes reached ˜60 mm³), mice were treated with (a) vehicle, (b) TY24118, (c) TY24122, (d) TY25366, or (e) TY25368. (Descriptions of the antibodies are provided in Table I above.) Antibodies were administered at 5 mg/kg or 1 mg/kg by intraperitoneal injection twice a week. Tumor growth was monitored twice a week and reported as the mean tumor volume±s.e.m. over time. As shown in FIGS. 18A and 18B, the parental anti-CD137 antibodies TY24118 and TY24122, which are not masked, showed similar efficacy at both high (5 mg/kg) and low (1 mg/kg) dose. The masked anti-CD137 antibody TY25368 showed similar efficacy with TY24118 and TY24122 both at high dose (5 mg/kg) and low dose (1 mg/kg). The masked anti-CD137 antibody TY25366 was less potent than TY24118 and TY24122 at both high (5 mg/kg) and low (1 mg/kg) dose.

### Exemplary Conclusions

TY25368 is an Fc-enhanced masked anti-CD137 with broad species cross-reactivity. TY25368 high masking efficiency and was conditionally activated to bind strongly to CD137 costimulatory receptor on activated T cells. TY25368 showed FcγR-dependent stimulation of a strong CD137 signaling, more potent than urelumab. TY25368 exhibited stronger anti-CD137 agonistic activity than urelumab for T cell activation in the presence of a primary stimulatory signal, while masked TY25368 had much lower activity. TY25368 demonstrated robust anti-tumor activity as single agent and cooperated with other immune checkpoint inhibitors including anti-PD-1 or anti-CTLA-4 to mediate enhanced antitumor efficacy. TY25368 is well-tolerated in rats and cynomolgus monkeys in nonclinical toxicology studies, with normal pharmacokinetic behaviors and minimal activation in circulation.

### Example 20: Anti-Tumor Efficacy of TY25368 in Combination with TY27151 in a Murine MC38-HER2-B7H3 Colon Adenocarcinoma Model

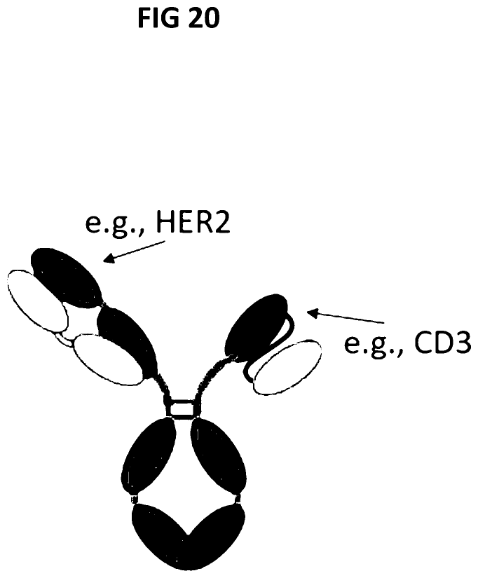

C57BL/6-hCD3e mice (n=6 per group, female, 9 weeks old) were inoculated subcutaneously with MC38-HER2-B7H3 murine colon adenocarcinoma cells. C57BL/6-hCD3e mice, which are derived from C57BL/6, have the human CD3E gene knocked in. Such mice express hCD3ε on their T cells. The MC38-HER2-B7H3 cell line is derived from the MC38 cell line and has been engineered to overexpress human HER2 (“hHER2”) and human B7H3 (“hB7H3”). When tumors were established (i.e., when tumor volumes reached (˜105 mm³), mice were treated with (a) vehicle, (b) TY25368, (c) TY27151, or (d) TY25368 and TY27151. Antibodies were each administered at 5 mg/kg by intraperitoneal injection twice a week. A description of TY25368 is provided in Table I above. TY27151 is a bispecific T-cell engager (TCE) construct that binds HER2 and CD3. Both the anti-CD3 and the anti-HER2 arms of TY27151 comprise a masking moiety and linkage unit that comprises a protease cleavage site. The amino acid sequences of the heavy and light chains of TY27151 are shown in Table L below. The masking peptides of TY27151 are in bold type. The masking sequences are underlined, and the linkage units are in italic type. TY27151 comprises three polypeptide chains: an anti-HER2 antibody heavy chain, and anti-HER2 antibody light chain, and an ScFv-Fc fusion polypeptide wherein the C-terminus of the ScFv is fused to the N-terminus of the Fc domain. The anti-HER2 antibody heavy chain and the anti-HER2 antibody light chain associate to form and anti-HER2 binding arm. The ScFv-Fc fusion polypeptide binds CD3. The Fc domain of the anti-HER2 binding arm and the Fc of the anti-CD3 ScFv-Fc fusion polypeptide dimerize to form the TCE. See, e.g., FIG. 20 for a schematic representation of TY27151. Additional details about TY27151 are provided in WO 2022/170740, the contents of which are incorporated herein by reference in their entirety.

TABLE L
Heavy Chain and Light Chain Amino Acid Sequences of TY27151

| | |
|---|---|
| Anti-HER2 | ESDACDADPFDCQAGGGPLGLAGSGGSDIQMTQSPSSLSASVGDRVTITCRASQDV |
| Light Chain | NTAVAWYQQKPGKAPKLLIYSASFLYSGVPSRFSGSRSGTDFTLTISSLQPEDFAT |
| | YYCQQHYTTPPTFGQGTKVEIKRTVAAPSVFIFPPSDEQLKSGTASVVCLLNNFYP |
| | REAKVQWKVDNALQSGNSQESVTEQDSKDSTYSLSSTLTLSKADYEKHKVYACEVT |
| | HQGLSSPVTKSFNRGEC (SEQ ID NO: 124) |
| Anti-HER2 | EVQLVESGGGLVQPGGSLRLSCAASGFNIKDTYIHWVRQAPGKGLEWVARIYPTNG |
| Heavy Chain | YTRYADSVKGRFTISADTSKNTAYLQMNSLRAEDTAVYYCSRWGGDGFYAMDYWGQ |
| | GTLVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNW |
| | YVDGVEVHNAKTKPREEQYASTYRVVSVLTVLHQDWLNGKEYKCKVSNKALPAPIE |
| | KTISKAKGQPREPQVYTLPPSRKKLTKNQVKLTCLVKGFYPSDIAVEWESNGQPEN |
| | NYKTTPPVLDCDGSFFLYSKLTVDKSRWQQGNVFSCSVMHEALHNHYTQKSLSLSP |
| | G (SEQ ID NO: 125) |
| Anti-CD3 | EVGSYPYDDPDCPSHESDCDQGGGPLGLAGSGGSQAVVTQEPSLTVSPGGTVTLTC |
| ScFv-Fc | GSSTGAVTTSNYPNWVQQKPGQAPRGLIGGTNKRAPGVPARFSGSLLGGKAALTLS |
| | GAQPEDEAEYYCALWYSNLWVFGGGTKLTVLGGGGSGGGGSGGGGSGGGGSEVQLV |
| | ESGGGLVQPGGSLRLSCAASGFTFNTYAINWVRQAPGKGLEWVGRIRSKYNNYATY |
| | YAESVKGRFTISRDDSKNTLYLQINSLRAEDTAVYYCVRHGNFGTSYVSWFAYWGQ |
| | GTLVTVSSEPKSSDKTHTCPPCPAPELLGGPSVFLFPPKPKDTLMISRTPEVTCVV |
| | VDVSHEDPEVKFNWYVDGVEVHNAKTKPREEQYASTYRVVSVLTVLHQDWLNGKEY |
| | KCKVSNKALPAPIEKTISKAKGQPREPQVYTDPPSRDELTKNQVSLTCLVDGFYPS |
| | DIAVEWESNGQPENCYKTTPPVLDSDGSFFLYSKLTVDKSRWQQGNVFSCSVMHEA |
| | LHNHYTQDSLSLSPG (SEQ ID NO: 126) |

*Masking sequences are in bold underlined type; linkage units are in bold italic type

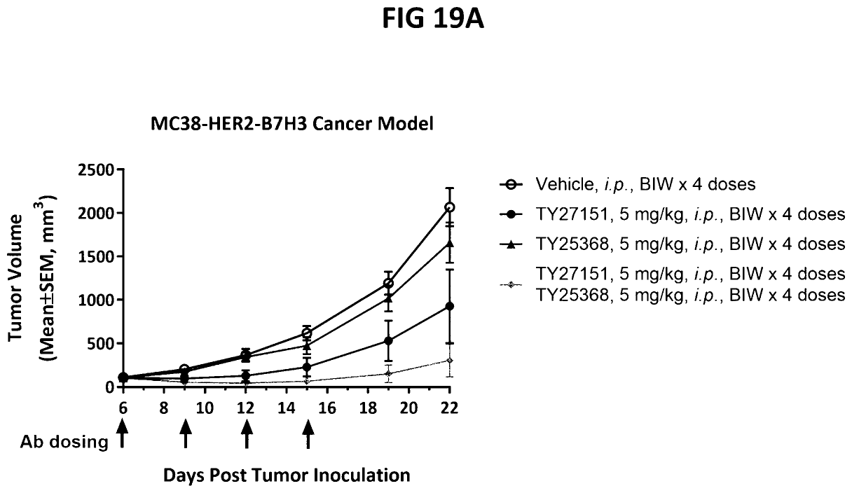

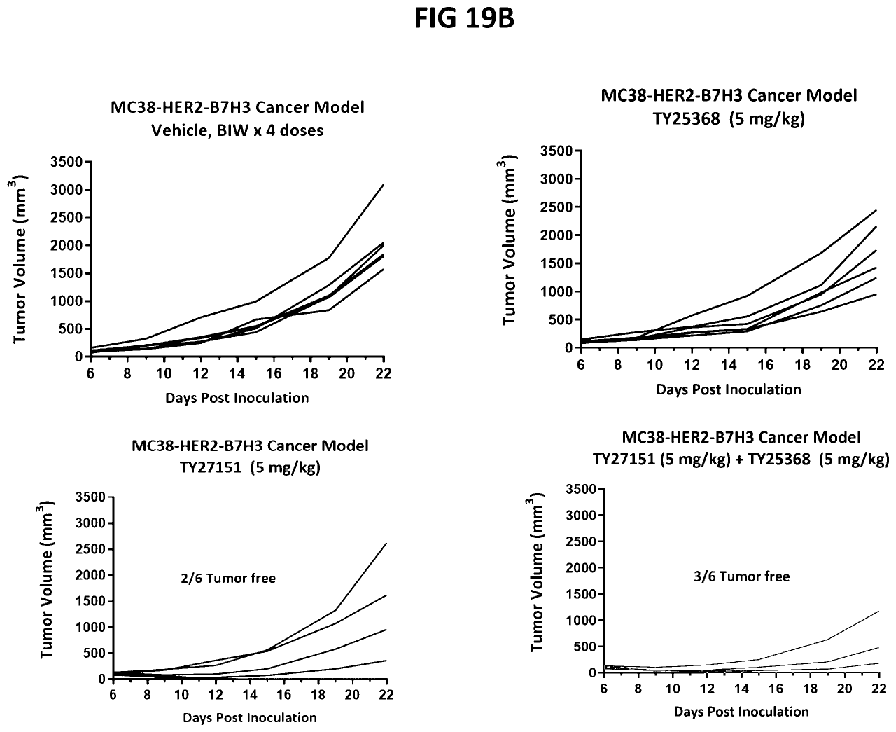

When tumors were established, treatment began with Vehicle, TY25368 (5 mg/kg), HER2×CD3 dual-masked bispecific antibody TY27151 (5 mg/kg), or the combination, by intraperitoneal injection, twice a week. Tumor growth was monitored twice a week and reported as the mean tumor volume±s.e.m. over time. As shown in FIG. 19A, TY25368 showed very weak efficacy as single agent, with TGI at 19% on Day 22. TY27151 demonstrated moderate efficacy as single agent, with TGI at 54% and with 2/8 tumor free mice on Day 22. Combination treatment with TY25368 and TY27151 resulted in significantly enhanced antitumor efficacy, with TGI at 85% and 3/8 tumor free mice on Day 22. Tumor growth curves of individual mice in each of treatment groups (a), (b), (c), and (d) are shown in FIG. 19B.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

What is claimed is:

1. A masked antibody comprising a masking peptide (MP) and an antibody that binds human CD137,
wherein the antibody comprises a heavy chain comprising a heavy chain variable region (VH) and a light chain comprising a light chain variable region (VL);
wherein the MP is linked to an N-terminus of the VL, wherein the MP comprises, from N-terminus to C-terminus, a masking unit (MU) and a linkage unit (LU), and wherein the MU comprises an amino acid sequence selected from the group consisting of SEQ ID NOS: 1-7; and
wherein the VH comprises a CDR-H1 set forth in TGGVGVG (SEQ ID NO: 36), a CDR-H2 set forth in LIDWADDKYYSPSLKS (SEQ ID NO: 37), and CDR-H3 set forth in GGSDTVIGDWFAY (SEQ ID NO: 38), and wherein the VL comprises a CDR-L1 set forth in RASQSIGSYLA (SEQ ID NO: 39), a CDR-L2 set forth in DASNLET (SEQ ID NO: 40), and a CDR-L3 set forth in QQGYYLWT (SEQ ID NO: 41).

2. The masked antibody of claim 1, wherein the MP further comprises an N-terminal unit (NU) linked to the N-terminal of the MU.

3. The masked antibody of claim 2, wherein the N-terminal unit is about 1-10 amino acid residues long.

4. The masked antibody of claim 3, wherein the N-terminal unit comprises E or EVGSY (SEQ ID NO: 77).

5. The masked antibody of any one of claims 1-4, wherein the LU comprises a first cleavage site.

6. The masked antibody of claim 5, wherein the first cleavage site is a protease cleavage site for a protease selected from the group consisting of urokinase-type plasminogen activator/uPA, matrix metalloproteinase-1/MMP-1, MMP-2, MMP-3, MMP-8, MMP-9, MMP-14, Tobacco Etch Virus protease/TEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE.

7. The masked antibody of claim 5, wherein the first cleavage site is a protease cleavage site for urokinase-type plasminogen activator/uPA or MMP-9.

8. The masked antibody of claim 5 or 6, wherein the LU further comprises a first linker (L₁).

9. The masked antibody of any one of claims 5-7, wherein the LU further comprises a second cleavage site.

10. The masked antibody of claim 8, wherein the second cleavage site is C-terminal to the L₁.

11. The masked antibody of 9 or 10, wherein the second cleavage site is a protease cleavage site for a protease selected from the group consisting of urokinase-type plasminogen activator/uPA, matrix metalloproteinase-1/MMP-1, MMP-2, MMP-3, MMP-8, MMP-9, MMP-14, Tobacco Etch Virus protease/TEV protease, plasmin, Thrombin, Factor X, PSA, PSMA, Cathepsin D, Cathepsin K, Cathepsin S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE.

12. The masked antibody of claim 11, wherein the second cleavage site is a protease cleavage site for urokinase-type plasminogen activator/uPA or MMP-9.

13. The masked antibody of any one of claims 9-12 wherein the first and second cleavage sites are the same.

14. The masked antibody of any one of claims 9-12 wherein the first and second cleavage sites are different.

15. The masked antibody of any one of claims 10-14, wherein the LU further comprises a second linker (L₂).

16. The masked antibody of any one of claims 5-15, wherein the LU comprises an amino acid sequence selected from the group consisting of SEQ ID NOs: 8-16.

17. The masked antibody of any one of claims 1-16, wherein the masking peptide comprises any one of SEQ ID NOs: 17-35.

18. The masked antibody of claim 17, wherein the masking peptide comprises SEQ ID NO: 34.

19. The masked antibody of any one of claims 1-18, wherein the antibody comprises a VH set forth in SEQ ID NO: 52 and a VL set forth in SEQ ID NO: 53.

20. The masked antibody of claim 18 and 19, wherein the VH comprises SEQ ID NO: 52 and the VL comprises SEQ ID NO: 58.

21. The masked antibody of any one of claims 1-20, wherein the masked antibody is a full length antibody comprising an Fc region.

22. The masked antibody of claim 21, wherein the Fc region is a human IgG Fc region or a variant thereof.

23. The masked antibody of claim 22, wherein the human IgG Fc region or variant thereof is a human IgG1, Fc region, a human IgG2 Fc region, a human IgG4 Fc region, or a variant of any one of the preceding.

24. The masked antibody of claim 23, comprising a variant of a human IgG1 Fc region that comprises substitution(s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D, and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index.

25. The masked antibody of claim 24, comprising a variant of a human IgG1 Fc region that comprises S267E and L328F substitutions, wherein amino acid numbering is according to the EU index.

26. The masked antibody of claim 25, comprising an Fc region that comprises SEQ ID NO:
113 or SEQ ID NO: 114.

27. The masked antibody of claim any one of claims 18-26, comprising the masking peptide set forth in SEQ ID NO: 34, the antibody heavy chain variable domain set forth in SEQ ID NO: 52 and an antibody light chain variable domain set forth in SEQ ID NO: 53, and a human IgG1 Fc region variant comprising S267E and L328F substitutions, wherein amino acid numbering is according to the EU index.

28. The masked antibody of claim 27, comprising a heavy chain comprising SEQ ID NO: 94 or 95 and a light chain comprising SEQ ID NO: 96.

29. The masked antibody of claim 23, comprising a variant of a human IgG4 Fc region that comprises substitution(s) selected from the group consisting of: G236D; L328F; S239D; S267E; G236D and S267E; S239D and S267E; S267E and L328S; and S267E and L328F; E233D and P238D; G237D and P238D; H268D and P238D; P271G and P238D; A330R and P238D; E233D, P238D, and A330R; E233D, P231G, P238D, and A330R; G237D, H268D, P238D, and P271G; G237D, P238D, P271G, and A330R; E233D, H268D, P238D, P271G, and A330R; G237D, H268D, P238D, P271G, and A330R; and E233D, G237D, P238D, H268D, P271G and A330R; S2657A; T437R; K248E; and T437R and K248E, wherein amino acid numbering is according to the EU index.

30. The masked antibody of claim 29, comprising a variant of a human IgG4 Fc region that comprises S267E and L328F substitutions, wherein amino acid numbering is according to the EU index.

31. The masked antibody of claim 30, comprising an Fc region that comprises SEQ ID NO: 117 or 118.

32. The masked antibody of any one of claims 18-23 and 29-31, comprising a masking peptide of SEQ ID NO: 34, an antibody heavy chain variable domain set forth in SEQ ID NO: 52 and an antibody light chain variable domain set forth in SEQ ID NO: 53, and a human IgG4 Fc region variant comprising S267E and L328F substitutions, wherein amino acid numbering is according to the EU index.

33. The masked antibody of claim 32, comprising a heavy chain comprising SEQ ID NO: 92 or 93 and a light chain comprising SEQ ID NO 96.

34. The masked antibody of any one of claims 1-20, wherein the antibody is an antibody fragment selected from the group consisting of: a Fab, a Fab′, a Fab′-SH, a F(ab′)2, an Fv, an scFv, an (scFv)2, a linear antibody, a single-chain antibody, single domain antibody (nanobody) VHH, a minibody, or a diabody.

35. One or more polynucleotides encoding the masked antibody of any one of claims 1-34.

36. A recombinant vector comprising the one or more polynucleotides of claim 35.

37. A host cell comprising a vector of claim 36.

38. A method of producing a masked antibody, comprising culturing the host cell of claim 37 under appropriate conditions to cause expression of the masked antibody and recovering the masked antibody.

39. A method of treating cancer in a subject, comprising administering to the subject an effective amount of the masked antibody of any one of claims 1-34.

40. The method of claim 39, wherein the cancer is solid tumor cancer.

41. The method of claim 40, wherein the solid tumor is breast cancer, liver cancer, colorectal cancer, or colon cancer r.

42. A method of treating cancer in a subject, comprising administering to the subject an effective amount of the masked antibody of any one of claims 1-34 and an effective amount of an anti-PD-1 antibody.

43. A method of treating cancer in a subject, comprising administering to the subject an effective amount of the masked antibody of any one of claims 1-34 and an effective amount of an anti-CTLA4 antibody.

44. A method of treating cancer in a subject, comprising administering to the subject an effective amount of the masked antibody of any one of claims 1-34 and an effective amount of a bispecific antibody that binds HER2 and CD3.

45. A method of treating cancer in a subject, comprising administering to the subject an effective amount of the masked antibody of any one of claims 1-34 and effective amount of a bispecific T cell engager (TCE) that targets CD3 and an antigen expressed on the surface of a solid tumor cancer cell.

46. The method of claim 45, wherein the antigen expressed on the surface of a solid tumor cancer cell is HER2.

47. The method of claim 45 or 46, wherein the TCE comprises three polypeptide chains, wherein a first polypeptide chain comprises an antibody heavy chain, a second polypeptide chain comprises an antibody light chain, and a third polypeptide chain comprises an scFv-Fc domain fusion wherein the C-terminus of the ScFv is fused to the N-terminus of the Fc domain, wherein the first and second polypeptide chains associate to from a HER-2 binding arm, wherein the third polypeptide chain binds CD3, and wherein the Fc domain of the anti-HER2 binding arm and the Fc of the third polypeptide chain dimerize to form the TCE.

48. The method of claim 47, wherein the first polypeptide chain comprises SEQ ID NO: 125, the second polypeptide chain comprises SEQ ID NO: 124, and the third polypeptide chain comprises SEQ ID NO: 126

49. The method of any one of claims 42-47, wherein the cancer is solid tumor.

50. The method of claim 48, wherein the solid tumor is colon cancer, breast cancer, liver cancer, colorectal cancer, or colon cancer.

51. A kit comprising the masked antibody of any one of claims 1-34 for treating an individual having cancer according to the method of any one of claims 39-41.

52. A kit comprising the masked antibody of any one of claims 1-34 for use in combination with an anti-PD-1 antibody for treating an individual having cancer according to the method any one of claims 42 and 46-47.

53. A kit comprising the masked antibody of any one of claims 1-36 for use in combination with an anti-CTLA4 antibody for treating an individual having cancer according to the method of any one of claims 43 and 48-49.

54. A kit comprising the masked antibody of any one of claims 1-36 for use in combination with a bispecific antibody that binds HER2 and CD3 for treating an individual having cancer according to the method of any one of claims 44 and 48-49.

55. A kit comprising the masked antibody of any one of claims 1-36 for use in combination with bispecific T cell engager that targets CD3 and an antigen expressed on the surface of a solid tumor cancer cell for treating an individual having cancer according to the method of any one of claims 45-49.


SEQUENCE LISTING (128 sequences)

SEQ ID NO: 1 - protein, 16 aa, synthetic construct
PTDLDACADAPNHCHF

SEQ ID NO: 2 - protein, 16 aa, synthetic construct
SSTHAHCHHSPANCIS

SEQ ID NO: 3 - protein, 16 aa, synthetic construct
DTDYDFCPILRHRCDS

SEQ ID NO: 4 - protein, 16 aa, synthetic construct
NDYNYHCKWRPSRCHN

SEQ ID NO: 5 - protein, 16 aa, synthetic construct
YHDYDDCRVLPRRCFN

SEQ ID NO: 6 - protein, 16 aa, synthetic construct
SNNFASCLWRHRSCAD

SEQ ID NO: 7 - protein, 16 aa, synthetic construct
TDNYDYCPRLRRKCYH

SEQ ID NO: 8 - protein, 20 aa, synthetic construct
SGRSAGGGGTPLGLAGSGGS

SEQ ID NO: 9 - skipped

SEQ ID NO: 10 - protein, 10 aa, synthetic construct
PLGLAGSGGS

SEQ ID NO: 11 - protein, 12 aa, synthetic construct
GGGPLGLAGGGS

SEQ ID NO: 12 - protein, 11 aa, synthetic construct
GGGPLGLAGGS

SEQ ID NO: 13 - protein, 12 aa, synthetic construct
SGGGPLGLAGGS

SEQ ID NO: 14 - protein, 12 aa, synthetic construct
SGRGPLGLAGGS

SEQ ID NO: 15 - protein, 13 aa, synthetic construct
SGRGGPLGLAGGS

SEQ ID NO: 16 - protein, 14 aa, synthetic construct
SGRGGGPLGLAGGS

SEQ ID NO: 17 - protein, 41 aa, synthetic construct
EVGSYPTDLDACADAPNHCHFSGRSAGGGGTPLGLAGSGGS

SEQ ID NO: 18 - protein, 41 aa, synthetic construct
EVGSYSSTHAHCHHSPANCISSGRSAGGGGTPLGLAGSGGS

SEQ ID NO: 19 - protein, 41 aa, synthetic construct
EVGSYDTDYDFCPILRHRCDSSGRSAGGGGTPLGLAGSGGS

SEQ ID NO: 20 - protein, 41 aa, synthetic construct
EVGSYNDYNYHCKWRPSRCHNSGRSAGGGGTPLGLAGSGGS

SEQ ID NO: 21 - protein, 41 aa, synthetic construct
EVGSYYHDYDDCRVLPRRCFNSGRSAGGGGTPLGLAGSGGS

SEQ ID NO: 22 - protein, 41 aa, synthetic construct
EVGSYSNNFASCLWRHRSCADSGRSAGGGGTPLGLAGSGGS

SEQ ID NO: 23 - protein, 41 aa, synthetic construct
EVGSYTDNYDYCPRLRRKCYHSGRSAGGGGTPLGLAGSGGS

SEQ ID NO: 24 - protein, 31 aa, synthetic construct
EVGSYDTDYDFCPILRHRCDSPLGLAGSGGS

SEQ ID NO: 25 - protein, 41 aa, synthetic construct; identical to SEQ ID NO: 19

SEQ ID NO: 26 - protein, 31 aa, synthetic construct
EVGSYNDYNYHCKWRPSRCHNPLGLAGSGGS

SEQ ID NO: 27 - protein, 41 aa, synthetic construct; identical to SEQ ID NO: 20

SEQ ID NO: 28 - protein, 31 aa, synthetic construct
EVGSYTDNYDYCPRLRRKCYHPLGLAGSGGS

SEQ ID NO: 29 - protein, 41 aa, synthetic construct; identical to SEQ ID NO: 23

SEQ ID NO: 30 - protein, 33 aa, synthetic construct
EVGSYTDNYDYCPRLRRKCYHGGGPLGLAGGGS

SEQ ID NO: 31 - protein, 32 aa, synthetic construct
EVGSYPTDLDACADAPNHCHFGGGPLGLAGGS

SEQ ID NO: 32 - protein, 33 aa, synthetic construct
EVGSYPTDLDACADAPNHCHFSGGGPLGLAGGS

SEQ ID NO: 33 - protein, 33 aa, synthetic construct
EVGSYPTDLDACADAPNHCHFSGRGPLGLAGGS

SEQ ID NO: 34 - protein, 34 aa, synthetic construct
EVGSYPTDLDACADAPNHCHFSGRGGPLGLAGGS

SEQ ID NO: 35 - protein, 35 aa, synthetic construct
EVGSYPTDLDACADAPNHCHFSGRGGGPLGLAGGS

SEQ ID NO: 36 - protein, 7 aa, synthetic construct
TGGVGVG

SEQ ID NO: 37 - protein, 16 aa, synthetic construct
LIDWADDKYYSPSLKS

SEQ ID NO: 38 - protein, 13 aa, synthetic construct
GGSDTVIGDWFAY

SEQ ID NO: 39 - protein, 11 aa, synthetic construct
RASQSIGSYLA

SEQ ID NO: 40 - protein, 7 aa, synthetic construct
DASNLET

SEQ ID NO: 41 - protein, 8 aa, synthetic construct
QQGYYLWT

SEQ ID NO: 42 - protein, 7 aa, synthetic construct
TSGVGVG

SEQ ID NO: 43 - protein, 16 aa, synthetic construct
LIDWDDDKYYSPSLKS

SEQ ID NO: 44 - protein, 13 aa, synthetic construct
GGSDTVLGDWFAY

SEQ ID NO: 45 - protein, 11 aa, synthetic construct
RASQSVSPYLA

SEQ ID NO: 46 - protein, 7 aa, synthetic construct
DASSLES

SEQ ID NO: 47 - protein, 8 aa, synthetic construct
QQGYSLWT

SEQ ID NO: 48 - protein, 6 aa, synthetic construct
SGHYWA

SEQ ID NO: 49 - protein, 17 aa, synthetic construct
SISGYGSTTYYADSVKG

SEQ ID NO: 50 - protein, 13 aa, synthetic construct
GGSDAVLGDWFAY

SEQ ID NO: 51 - protein, 11 aa, synthetic construct
RASQGIGSFLA

SEQ ID NO: 52 - protein, 123 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSS

SEQ ID NO: 53 - protein, 107 aa, synthetic construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 54 - protein, 123 aa, synthetic construct
EVQLVESGGGLVQPGGSLRLSCAASGFSLSTSGVGVGWIRQAPGKGLEWLALIDWDDDKYYSPSLKSRLTISRDNSKNTLYLQLNSLRAEDTAVYYCARGGSDTVLGDWFAYWGQGTLVTVSS

SEQ ID NO: 55 - protein, 107 aa, synthetic construct
DIQLTQSPSSLSASVGDRVTITCRASQSVSPYLAWYQQKPGKAPKLLIYDASSLESGVPSRFSGSGSGTDFTLTISSLQPEDFATYYCQQGYSLWTFGQGTKVEIKR

SEQ ID NO: 56 - protein, 123 aa, synthetic construct
EVQLVESGGGLVQPGGSLRLSCAASGYSITSGHYWAWIRQAPGKGLEWVSSISGYGSTTYYADSVKGRFTISRDNSKNTLYLQLNSLRAEDTAVYYCARGGSDAVLGDWFAYWGQGTLVTVSS

SEQ ID NO: 57 - protein, 107 aa, synthetic construct
DIQLTQSPSSLSASVGDRVTITCRASQGIGSFLAWYQQKPGKAPKLLIYDASNLETGVPSRFSGSGSGTDFTLTISSLQPEDFATYYCQQGYYLWTFGQGTKVEIKR

SEQ ID NO: 58 - protein, 141 aa, synthetic construct
EVGSYPTDLDACADAPNHCHFSGRGGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 59 - protein, 148 aa, synthetic construct
EVGSYPTDLDACADAPNHCHFSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 60 - protein, 148 aa, synthetic construct
EVGSYSSTHAHCHHSPANCISSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 61 - protein, 148 aa, synthetic construct
EVGSYDTDYDFCPILRHRCDSSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 62 - protein, 148 aa, synthetic construct
EVGSYNDYNYHCKWRPSRCHNSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 63 - protein, 148 aa, synthetic construct
EVGSYYHDYDDCRVLPRRCFNSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 64 - protein, 148 aa, synthetic construct
EVGSYSNNFASCLWRHRSCADSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 65 - protein, 148 aa, synthetic construct
EVGSYTDNYDYCPRLRRKCYHSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 66 - protein, 138 aa, synthetic construct
EVGSYDTDYDFCPILRHRCDSPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 67 - protein, 148 aa, synthetic construct; identical to SEQ ID NO: 61

SEQ ID NO: 68 - protein, 138 aa, synthetic construct
EVGSYNDYNYHCKWRPSRCHNPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 69 - protein, 148 aa, synthetic construct; identical to SEQ ID NO: 62

SEQ ID NO: 70 - protein, 138 aa, synthetic construct
EVGSYTDNYDYCPRLRRKCYHPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 71 - protein, 148 aa, synthetic construct; identical to SEQ ID NO: 65

SEQ ID NO: 72 - protein, 140 aa, synthetic construct
EVGSYTDNYDYCPRLRRKCYHGGGPLGLAGGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 73 - protein, 139 aa, synthetic construct
EVGSYPTDLDACADAPNHCHFGGGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 74 - protein, 140 aa, synthetic construct
EVGSYPTDLDACADAPNHCHFSGGGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 75 - protein, 139 aa, synthetic construct
EVGSYPTDLDACADAPNHCHSGRGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 76 - protein, 142 aa, synthetic construct
EVGSYPTDLDACADAPNHCHFSGRGGGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKR

SEQ ID NO: 77 - protein, 5 aa, synthetic construct
EVGSY

SEQ ID NO: 78 - protein, 5 aa, synthetic construct
GGGGT

SEQ ID NO: 79 - protein, 4 aa, synthetic construct
SGGS

SEQ ID NO: 80 - protein, 4 aa, synthetic construct
GGSG

SEQ ID NO: 81 - protein, 5 aa, synthetic construct
GGSGG

SEQ ID NO: 82 - protein, 5 aa, synthetic construct
GSGSG

SEQ ID NO: 83 - protein, 5 aa, synthetic construct
GSGGG

SEQ ID NO: 84 - protein, 5 aa, synthetic construct
GGGSG

SEQ ID NO: 85 - protein, 5 aa, synthetic construct
GSSSG

SEQ ID NO: 86 - protein, 5 aa, synthetic construct
SGRSA

SEQ ID NO: 87 - protein, 6 aa, synthetic construct
PLGLAG

SEQ ID NO: 88 - protein, 450 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 449 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 90 - protein, 453 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 452 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 450 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVEQEDPEVQFNWYVDGVEVHNAKTKPREEQFNSTYRVVSVLTVLHQDWLNGKEYKCKVSNKGFPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 449 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVEQEDPEVQFNWYVDGVEVHNAKTKPREEQFNSTYRVVSVLTVLHQDWLNGKEYKCKVSNKGFPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 94 - protein, 453 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 452 aa, synthetic construct
EVQLVESGGGLVQPGGSLRLSCAASGFSLSTGGVGVGWIRQAPGKGLEWLALIDWADDKYYSPSLKSRLTISRDNSKNTLYLQLNSLRAEDTAVYYCARGGSDTVIGDWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 247 aa, synthetic construct
EVGSYPTDLDACADAPNHCHFSGRGGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 254 aa, synthetic construct
EVGSYPTDLDACADAPNHCHFSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 254 aa, synthetic construct
EVGSYDTDYDFCPILRHRCDSSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 244 aa, synthetic construct
EVGSYNDYNYHCKWRPSRCHNPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 254 aa, synthetic construct
EVGSYNDYNYHCKWRPSRCHNSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 244 aa, synthetic construct
EVGSYTDNYDYCPRLRRKCYHPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 254 aa, synthetic construct
EVGSYTDNYDYCPRLRRKCYHSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 244 aa, synthetic construct
EVGSYDTDYDFCPILRHRCDSPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 246 aa, synthetic construct
EVGSYTDNYDYCPRLRRKCYHGGGPLGLAGGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 245 aa, synthetic construct
EVGSYPTDLDACADAPNHCHFGGGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 246 aa, synthetic construct
EVGSYPTDLDACADAPNHCHFSGGGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 246 aa, synthetic construct
EVGSYPTDLDACADAPNHCHFSGRGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 5 aa, synthetic construct
GGGGS

SEQ ID NO: 109 - protein, 213 aa, synthetic construct
DIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - skipped

SEQ ID NO: 111 - protein, 330 aa, Homo sapiens [sequence omitted]

SEQ ID NO: 112 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 117 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVEQEDPEVQFNWYVDGVEVHNAKTKPREEQFNSTYRVVSVLTVLHQDWLNGKEYKCKVSNKGFPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVEQEDPEVQFNWYVDGVEVHNAKTKPREEQFNSTYRVVSVLTVLHQDWLNGKEYKCKVSNKGFPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 119 - protein, 254 aa, synthetic construct; identical to SEQ ID NO: 98

SEQ ID NO: 120 - protein, 254 aa, synthetic construct
EVGSYYHDYDDCRVLPRRCFNSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 254 aa, synthetic construct
EVGSYSNNFASCLWRHRSCADSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 248 aa, synthetic construct
EVGSYPTDLDACADAPNHCHFSGRGGGPLGLAGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 254 aa, synthetic construct
EVGSYSSTHAHCHHSPANCISSGRSAGGGGTPLGLAGSGGSDIQLTQSPSSLSASVGDRVTITCRASQSIGSYLAWYQQKPGKAPKLLIYDASNLETGVPSRFSGSGSGTDFTLTISSLQPEDFATYYCQQGYYLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 241 aa, synthetic construct
ESDACDADPFDCQAGGGPLGLAGSGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKKLTKNQVKLTCLVKGFYPSDIAVEWESNGQPENNYKTTPPVLDCDGSFFLYSKLTVDKSRWQQGNVFSCSVMHEALHNHYTQKSLSLSPG

SEQ ID NO: 126 - protein, 519 aa, synthetic construct
EVGSYPYDDPDCPSHESDCDQGGGPLGLAGSGGSQAVVTQEPSLTVSPGGTVTLTCGSSTGAVTTSNYPNWVQQKPGQAPRGLIGGTNKRAPGVPARFSGSLLGGKAALTLSGAQPEDEAEYYCALWYSNLWVFGGGTKLTVLGGGGSGGGGSGGGGSGGGGSEVQLVESGGGLVQPGGSLRLSCAASGFTFNTYAINWVRQAPGKGLEWVGRIRSKYNNYATYYAESVKGRFTISRDDSKNTLYLQINSLRAEDTAVYYCVRHGNFGTSYVSWFAYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTDPPSRDELTKNQVSLTCLVDGFYPSDIAVEWESNGQPENCYKTTPPVLDSDGSFFLYSKLTVDKSRWQQGNVFSCSVMHEALHNHYTQDSLSLSPG

SEQ ID NO: 127 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 128 - protein, 7 aa, synthetic construct
GGGGSSS